(19) United States
(12) Patent Application Publication
(10) Pub. No.: US 2023/0065543 A1
(43) Pub. Date: Mar. 2, 2023

(54) SCORING CAREGIVERS AND TRACKING THE DEVELOPMENT OF CARE RECIPIENTS AND RELATED SYSTEMS AND METHODS

(71) Applicant: Littlebird Connected Care, Inc., Fall City, WA (US)

(72) Inventors: Monica Plath, Yakima, WA (US); Gadi Amit, San Mateo, CA (US)

(21) Appl. No.: 17/901,740

(22) Filed: Sep. 1, 2022

Related U.S. Application Data
(60) Provisional application No. 63/239,865, filed on Sep. 1, 2021.
(60) Provisional application No. 63/247,692, filed on Sep. 23, 2021.

(51) Int. Cl. G06Q 10/06; G16H 40/20; G16H 40/67

(52) U.S. Cl. CPC G06Q 10/06398; G16H 40/20; G16H 40/67

(57) ABSTRACT

Systems and methods for reviewing, rating, and/or otherwise scoring caregivers as well as monitoring the development and wellbeing of care recipients are disclosed herein. In some implementations, the system includes controlling persons, supervising persons (e.g., caregivers), supervised persons (e.g., care recipients), and one or more remote servers in communication with each of the controlling persons, the supervising persons, and the care recipients. Each of the controlling persons and the supervising persons can have a personal electronic. The supervised person can have a wearable electronic device. The electronic devices communicate evaluation data to the remote server related to the physical, emotional, cognitive, and/or social development of the supervised person. The wearable device communicate bioindicator data to the remote server that corroborates and/or contradicts the evaluation data. The remote server uses the data to score the supervising persons and evaluate the developmental status of the supervised person.

### RELATED APPLICATIONS

The present application claims the benefit of U.S. Provisional Pat. Application No. 63/239,865 by Monica Plath, filed Sep. 1, 2021, and U.S. Provisional Pat. Application No. 63/247,692 by Monica Plath, filed Sep. 23, 2021, the entirety of each of which are incorporated herein by reference. The present application is also related to U.S. Provisional Pat. Application No. 63/260,440 by Monica Plath, filed Aug. 19, 2021, and U.S. Pat. Application No. 17/891,781 by Monica Plath, filed Aug. 19, 2022, the disclosures of each of which are incorporated herein in their entireties by reference.

### TECHNICAL FIELD

The present disclosure is generally related to monitoring the development of one or more persons, as well as scoring the caregivers associated with the one or more persons. In particular, the present technology relates to wearable devices and related systems for evaluating the developmental status of supervised persons and scoring the impact that care providers have on the developmental status of the supervised persons.

### BACKGROUND

Monitoring and acting on the health and development of our loved ones is an important aspect of daily life. For example, we closely supervise our children’s development through their early years and continue to supervise and care for them as they grow older. Then, as our parents and other family members age, we become increasingly reinvolved in their daily lives, care, and wellbeing to help them maintain their lives as long as possible. For many of us, the supervision of our children, elderly family members, and other loved ones to monitor and improve their developmental status requires the assistance of caregivers to balance the supervision with busy work schedules and daily life. For example, American families spend upwards of forty billion dollars on childcare in a typical year, with more than half of American families with a child under the age of five paying for some amount of childcare. However, existing systems for evaluating the quality and developmental value of the care provided are outdated. For example, it is difficult to understand which caregivers best provide an environment for physical, emotional, cognitive, and/or social development to the children under their supervision. Similarly, it is difficult to understand which caregivers best provide an environment for maintaining physical, emotional, cognitive, and/or social functioning to the elderly under their supervision. Meanwhile, it can be difficult for caregivers to spot and communicate underdevelopment in the children under their supervision and/or the deterioration of the elderly under their supervision. It is even more difficult to understand how the parents and caregivers can change their behaviors to help avoid and/or correct underdevelopment and/or deterioration.

### BRIEF DESCRIPTION OF THE DRAWINGS

FIG. 1 is a schematic view of a system for scoring supervising persons and tracking the development of supervised persons in accordance with some implementations of the present technology.

FIG. 2 is a network diagram of the system for scoring supervising persons and tracking the development of supervised persons in accordance with some implementations of the present technology.

FIG. 3 is a schematic diagram of a subsystem for a controlling person in the system of FIGS. 1 and 2 in accordance with some implementations of the present technology.

FIG. 4 is a schematic diagram of a subsystem for a supervising person in the system of FIGS. 1 and 2 in accordance with some implementations of the present technology.

FIG. 5 is a schematic diagram of a subsystem for a wearable device for use by a supervised person in the system of FIGS. 1 and 2 in accordance with some implementations of the present technology.

FIG. 6 is a schematic view of a series of rated interpersonal interactions in accordance with some implementations of the present technology.

FIG. 7A is a schematic view of a process for rating a supervised person after a single interpersonal interaction in accordance with some implementations of the present technology.

FIG. 7B is a schematic view of the process of FIG. 7A over a series of interpersonal interactions in accordance with some implementations of the present technology.

FIG. 8 is a flow diagram of a process for scoring a supervised person after a single interpersonal interaction in accordance with some implementations of the present technology.

FIG. 9 is a flow diagram of a process for monitoring and aiding the development of a supervised person interaction in accordance with some implementations of the present technology.

FIG. 10 is a schematic view of a process for rating a supervising person after one or more interpersonal interactions in accordance with some implementations of the present technology.

FIG. 11 is a flow diagram of a process for scoring a supervising person after a single interpersonal interaction in accordance with some implementations of the present technology.

FIG. 12 is a flow diagram of a process for rating a supervising person’s after multiple interpersonal interactions in accordance with some implementations of the present technology.

FIG. 13 is a schematic view of a subsystem for making recommendations regarding the developmental status of a supervised person in accordance with some implementations of the present technology.

FIG. 14 is a schematic view of a for developing a predictive model for monitoring the developmental status of supervised persons in accordance with some implementations of the present technology.

FIG. 15 is a flow diagram of a process for associatively linking data from one or more subsystems in accordance with some implementations of the present technology.

FIG. 16 is a flow diagram of a process for adapting a predictive model for a specific supervised person and applying the adapted predictive model in accordance with some implantations of the present technology.

FIG. 17 is a flow diagram of a process for training a predictive model to predict future health and developmental statuses of a supervised person in accordance with some implementations of the present technology.

FIG. 18 is a flow diagram of a process for aggregating and outputting data for use in researching human development in accordance with some implementations of the present technology.

FIG. 19 is a flow diagram of a process for using a predictive model to the target data of a supervised person in accordance with some implementations of the present technology in accordance with some implementations of the present technology.

FIG. 20 is a flow diagram of a process for updating a predictive model in accordance with some implementations of the present technology.

FIG. 21 is a block diagram showing some of the components typically incorporated in at least some of the computer systems and other devices on which the disclosed system operates in accordance with some implementations of the present technology.

FIG. 22 is a system diagram illustrating an example of a computing environment in which the disclosed system operates in some implementations of the present technology.

The drawings have not necessarily been drawn to scale. Similarly, some components and/or operations can be separated into different blocks or combined into a single block for the purpose of discussion of some of the implementations of the present technology. Moreover, while the technology is amenable to various modifications and alternative forms, specific implementations have been shown by way of example in the drawings and are described in detail below. The intention, however, is not to limit the technology to the particular implementations described.

Further, some of the drawings include depictions of examples of supervising persons and/or supervised persons in accordance with some implementations of the present technology. Although depicted primarily as a system for use with children, toddlers, and infants, one of skill in the art will understand that the technology is not so limited. For example, the systems and methods depicted in the drawings can also be used to aid and/or supplement the supervision of an elderly person, a person with a mental disability, and/or any other person that requires at least partial supervision over their daily activities. Accordingly, the scope of the technology is not confined to any subset of implementations depicted in the drawings.

### DETAILED DESCRIPTION

### Overview

Systems and methods for reviewing, rating, and/or otherwise scoring caregivers; reviewing, rating, and/or otherwise scoring care recipients to set a baseline for their behavior in interactions; and/or monitoring, tracking, and/or improving the development and wellbeing of care recipients are disclosed herein. In some implementations, the system includes subsystem(s) associated with one or more controlling persons, subsystem(s) associated with one or more caregivers (also referred to herein as “supervising persons” and/or “care providers”), wearable device(s) associated with one or more care recipients (also referred to herein as “supervised persons” and/or “care receivers”), and one or more remote servers (e.g., a cloud server or other remote server) in communication with each of the controlling persons, the care providers, and the supervised person. For example, each of the controlling persons and the supervising persons can have a personal electronic device (e.g., a smartphone, tablet, personal computer, personal assistant, wearable device, and the like); while the supervised person can have a wearable electronic device. The electronic devices allow the controlling persons and the supervising persons (referred to collectively herein as “responsible persons”) to actively communicate with the remote server(s) to upload data (“evaluation data”) related to the supervised person (e.g., evaluations of the physical, emotional, cognitive, and/or social development of the supervised person; reports on interactions with the supervised person; reports on daily events for the supervised person; and the like) that is accessibly stored in the remote server(s). In some implementations, the evaluation data is uploaded with a required security and/or permission level included. For example, uploading and/or viewing an evaluation of the emotional and/or mental development of a supervised person can be restricted to responsible persons with a preset connection security level and/or a preset permissions level (e.g., restricting uploading and reviewing evaluations of a child’s mental development to a head childcare provider and the child’s parents).

Meanwhile, the wearable device can include one or more sensors that measure bioindicators of the supervised person (e.g., heart rate, skin temperature, skin conductivity, movement, heart-rate variability, resting heart rate, sweat chemical composition, hydration levels, nervous system electrical signals, stress levels, air quality, UV exposure, environmental chemical exposures, environmental chemical sensitivity, blood oxygen and/or pulse oxygen, voice commands and/or other sounds, electrical activity of the heart (also referred to herein as cardiac system electrical signals), atmospheric pressure, pressure on the wearable device, orientation of the wearable device on the supervised person, strength and direction of electromagnetic fields around the supervised person, and the like) and one or more communication components to communicate data on the bioindicators (“bioindicator data”) to the controlling persons and/or the supervising persons and/or directly to the remote server(s). Once communicated out from the wearable device, the bioindicator data can be accessibly stored in the remote server(s).

In some implementations, the bioindicator data can supplement, expand on, confirm, corroborate, challenge, correct, and/or contradict the evaluation data from the responsible persons. Purely by way of example, a childcare provider can upload an evaluation indicating that a child under their supervision was generally calm and happy; and the bioindicator data can then corroborate (or contradict) the evaluation with biological data demonstrating that the child experienced relatively low (or high) levels of stress while under the supervision of the childcare provider. In some implementations, when the bioindicators contradict the evaluation data uploaded by a responsible person (e.g., heart rate and skin conductivity depart from a baseline to indicate stress), the remote server(s) prompts the responsible person to update their evaluation. In some cases, the prompt can cause the responsible person to provide an alternative, more accurate evaluation. In other cases, the prompt can cause the responsible person to explain the apparent contradiction (e.g., to explain that the relatively high stress levels are at least partially the result of a particularly irritable day for the child). In some implementations, the amount of corroboration and/or contradiction between the evaluation data and the bioindicator data can be recorded and used in various methods of the system (e.g., a consistent mismatch between evaluations from a childcare provider and the bioindicators can negatively affect a scoring of the childcare provider).

In some implementations, the evaluation data and the bioindicator data are combined as data on rated interpersonal interactions (“RIPI data”) between one or more supervising persons and one or more supervised persons. The RIPI data then be used to establish baselines for each of the supervising person(s) and the supervised person(s) (e.g., to establish that Child-A is generally shy around childcare providers while Child-B is very interactive with childcare providers); rate the developmental impact and/or quality of care provided by the supervising person(s) (referred to herein as a “RIPI score”); and/or evaluate and/or track the physical, emotional, cognitive, and/or social developmental status of the supervised person(s).

The RIPI data, baselines, RIPI scores, and/or developmental status of the supervised person(s) can then be reviewed and utilized by the controlling persons. As a result, for example, the system can allow parents to make more informed decisions about the childcare workers they entrust with the supervision of their toddlers. The RIPI data, baselines, RIPI scores, and/or developmental status of the supervised person(s) can also be reviewed and utilized by the supervising persons. For example, the system can provide childcare workers with detailed evaluations of their strengths and weaknesses, allowing them to take corrective actions to address their weaknesses and provide a higher quality of care to the toddlers under their supervision and/or to focus on specific developmental needs for the toddlers under their supervision.

In another example, the RIPI data, baselines, and/or developmental status can be used by one or more supervising persons before interacting with new supervised persons. Purely by way of example, a childcare provider can request the RIPI data, baselines, and/or developmental status in an application to the childcare provider. The RIPI data, baselines, and/or developmental status can then influence which applications the childcare provider accepts, which children are assigned to which supervising persons (e.g., to evenly distribute children among the supervising persons), and/or identify children for special focus (e.g., identify gifted children for advanced placements, identify children needing extra attention, and the like).

In a specific, non-limiting, example, the system can include two parents, a daycare service with multiple childcare providers, and one or more toddlers equipped with wearable devices. After various interactions (e.g., greeting the toddler(s), leaving the toddler(s) alone for a moment, providing a snack to the toddler(s), and/or separating the toddler(s) from the parents), the system (e.g., through a module in the remote server) can prompt the childcare providers to provide an evaluation of each toddler’s behavior. In some implementations, the evaluation is a form evaluation including various prompts that allow the childcare provider to quickly enter the evaluation. The prompts can be related to standardized developmental goals, classifications, milestones, and the like. Purely by way of example, the World Health Organization (“WHO”) has published an attachment classification framework that includes: secure; insecure-avoidant; insecure-ambivalent, anxious or resistant; and disorganized-disoriented.¹ The prompts can be related to evaluating the attachment classification of each of the toddlers, helping signal to the parents and/or childcare providers when some corrective action may be helpful to encourage the emotional and/or social development of their toddlers. In another example, the United States Center for Disease Control (“CDC”) has also identified a range of physical, emotional, and cognitive developmental milestones as well as expectations for when toddlers should achieve the milestones.² The prompts can be related to identifying when each of the toddlers achieves various milestones, helping signal to the parents and/or childcare providers when some corrective action may be helpful and/or when a toddler is ahead of expectations. In a related example related to the care of an elderly person, various research institutions have identified links between the physical, emotional, and/or cognitive status of elderly individuals and their long-term functional capacity and/or lifespan.³ The prompts can be related to identifying various physical and/or cognitive events that may indicate that a medical intervention is necessary to improve the supervise person’s long-term functional capacity and/or lifespan.

In some implementations, standardized developmental classifications can have an expected distribution of toddlers in each classification. Returning to the attachment classifications identified by the WHO, about 55% of toddlers are expected to be classified as secure; about 20% are expected to be classified as insecure-avoidant; about 15% are expected to be classified as insecure-ambivalent, anxious or resistant; and up to 8% are expected to be classified as disorganized-disoriented. The system can identify when the evaluations from a childcare provider deviate from the expected distribution by more than a predetermined amount (e.g., by more than one, two, three or any suitable number of standard deviations for the expected distribution). In some cases, the excessive deviations can indicate errors in how the childcare provider is evaluating the toddlers and/or attributes about the childcare provider that are causing the deviations (e.g., improving or negatively impacting child development). In some implementations, what qualifies as an excessive deviation is dependent on the type of evaluation and/or any underlying metrics. For example, a subjective assessment can be expected to have larger deviations than objective assessments. In another example, a type of evaluation with more established data can have a smaller threshold for excessive deviations than a type of evaluation with less established data. In some implementations, the range for excessive deviations is also dependent on the established data for the supervised person. In some implementations, the system can include one or more checks against the evaluations from the childcare providers to help ensure their accuracy.

For one accuracy check, for example, each toddler can be evaluated by different childcare providers, allowing the system to establish a baseline rating and/or average evaluation for each toddler. If a particular childcare provider provides evaluations that deviate from the baseline and/or average, the deviation may indicate some error in the evaluation. Purely by way of example, a particular child may have a baseline indicating that they are extremely shy. In this example, a childcare provider that evaluates the toddler as outgoing and interactive can be prompted to confirm their evaluation because it deviates from the baseline for the toddler. Additionally, or alternatively, the deviations can indicate that the childcare provider themselves are at least partially responsible for the deviations. For example, a childcare provider that consistently evaluates toddlers as shy and/or insecure can be identified as possibly making the toddlers under their supervision uncomfortable, allowing the system to discount their evaluations of the toddlers in updating their baseline and/or average.

In another accuracy check, the system can compare the evaluations against the bioindicator data from a wearable device of the toddler for consistency and/or contradictions. For example, an indication of elevated heart rate and/or stress signals (e.g., compared to baseline levels, increases during the interaction, and the like) can contradict an evaluation that the toddler was calm and happy during the relevant interaction(s). When a contradiction is identified, the childcare provider can be prompted to edit their evaluation(s) and/or explain the apparent contradiction(s). Purely by way of example, the seeming contradictions discussed above might be explained by a particularly bad day for the toddler (e.g., when they have not gotten enough sleep), the interaction taking place immediately after another event (e.g., an elevated heart rate immediately after playtime), and/or various other causal factors (e.g., toddler is experiencing a growth spurt). However, consistent contradictions can indicate that the childcare provider is evaluating the toddler inaccurately, allowing the system to discount and/or assign a lower weight to evaluations from that childcare provider.

In some implementations, the system scores and/or rates the childcare providers (e.g., via the RIPI scores) in addition to, or in alternative to, collecting and aggregating the evaluations of the toddlers. For example, the system can determine that a childcare provider that has consistent, accurate (or uncontradicted) positive evaluations of the toddlers under their care is positively impacting the toddlers and provide the childcare provider with a positive RIPI score. Conversely, the system can determine that a childcare provider is negatively impacting the toddlers under their care and provide the childcare provider with a negative RIPI score. In some implementations, the system establishes a baseline rating for the childcare provider before using their evaluations to assess their impact on toddlers. For example, a childcare provider may be a harsher (or lighter) evaluator without deviating from an expected distribution by more than the predetermined amount for the system to take corrective action. The system can then set a baseline expecting the harsher ratings from the childcare provider to avoid a false-negative evaluation of the childcare provider. Conversely, the system can set a baseline for a lighter evaluator to avoid a false-positive evaluation of the childcare provider.

In some implementations, the system can use the bioindicator data and/or other sources of RIPI data in setting and/or adjusting the RIPI score for the childcare provider. For example, consistent bioindicators of elevated stress levels around the childcare can negatively impact the RIPI score for the childcare provider. In another example, parents can provide data on interpersonal interactions with the childcare provider to provide an additional source of RIPI data.

In some implementations, the wearable device on the toddler can communicate updates on the physical, mental, and/or emotional indicators/status of the toddler throughout the day and/or the toddler’s location. To do so, the wearable device can communicate with an electronic device of any nearby responsible person (e.g., the electronic device of the childcare workers), a nearby beacon, one or more internet of things (IoT) devices in its vicinity, and/or directly to the remote server (e.g., over an internet or cellular connection). The status updates can allow the toddler’s parents to easily monitor the health and development of their child throughout the day, as well as the child’s physical location.

Further, the status updates can be timestamped, allowing the toddler’s parents (and/or the system) to cross-reference significant events with which childcare worker was supervising their toddler at the time. As a result, the parents can request additional information from that childcare worker, determine which childcare workers should (or should not) be trusted with the supervision of the toddler, and/or determine which childcare workers should (or should not) the toddler should spend time with. For example, a record indicating that the toddler was especially happy and/or mentally stimulated while under the supervision of a particular childcare worker can indicate that the toddler should spend additional time with that particular childcare worker. In an alternative example, a record indicating that the toddler was especially unhappy or experienced a significant event that is unaccounted for while under the supervision of a particular childcare worker may indicate that the particular childcare worker should not be trusted with the toddler.

In some implementations of the present technology, the remote server includes one or more components that automatically review the data (including data from the responsible persons and/or the wearable sensor) related to the supervised person. In doing so, the remote server can generate a report on the physical health, mental health, emotional health, and/or developmental status of the supervised person. For example, the remote server can indicate when a toddler may be getting sick, may not have had enough sleep, etc. Additionally, or alternatively, the remote server can generate recommendations to the responsible persons related to the physical health, mental health, emotional health, and/or developmental status of the supervised person. For example, the remote server can recommend additional cognitive activities to generate additional mental stimulation and development when detecting that a toddler has fallen behind predetermined milestones (e.g., the CDC-defined milestones for child development).

In some implementations, the system includes one or more controlling persons, one or more supervising persons (sometimes also referred to herein as “caregivers” and/or “care providers”), one or more supervised persons (sometimes also referred to herein as “care recipients” and/or “care receivers”), and one or more remote servers in communication with each of the controlling persons, the care providers, and the supervised person. For example, each of the controlling persons and the supervising persons can have a personal electronic device (e.g., a smartphone, tablet, personal computer, personal assistant, wearable device, and the like); while the supervised person can have a wearable electronic device. The electronic devices allow the controlling persons and the supervising persons (referred to collectively herein as “responsible persons”) to actively communicate with the remote server(s) to upload data (“developmental data”) related to the supervised person that is accessibly stored in the remote server(s). The developmental data can include assessments of the physical, emotional, cognitive, and/or social development of the supervised person; reports on the overall health of the supervised person; reports on the supervised person’s daily activities and/or experiences; observed developmental milestones; and the like. In some implementations, the developmental data is uploaded with a required security and/or permission level included. For example, uploading and/or viewing an evaluation of the emotional and/or mental development of a supervised person can be restricted to responsible persons with a preset connection security level and/or a preset permissions level (e.g., restricting uploading and reviewing evaluations of a child’s mental development to a head childcare provider and their parents).

Meanwhile, the wearable device can include one or more sensors that measure bioindicators of the supervised person (e.g., heart rate, skin temperature, skin conductivity, movement, heart-rate variability, resting heart rate, sweat chemical composition, hydration levels, nervous system electrical signals, stress levels, air quality, UV exposure, environmental chemical exposures, environmental chemical sensitivity, blood oxygen and/or pulse oxygen, voice commands and/or other sounds, electrical activity of the heart, atmospheric pressure, pressure on the wearable device, orientation of the wearable device on the supervised person, strength and direction of electromagnetic fields around the supervised person, and the like) and one or more communication components to communicate data one the bioindicators (“bioindicator data”) to the controlling persons and/or the supervising persons and/or directly to the remote server(s). Once communicated out from the wearable device, the bioindicator data can be accessibly stored in the remote server(s).

In some implementations, the bioindicator data can supplement, expand on, confirm, corroborate, challenge, correct, and/or contradict the developmental data from the responsible persons. Purely by way of example, a childcare provider can upload a report indicating that a child under their supervision was generally calm and happy; and the bioindicator data can then corroborate (or contradict) the report with biological data demonstrating that the child experienced relatively low (or high) levels of stress while under the supervision of the childcare provider (e.g., based on data reflecting an elevated heart rate and/or blood pressure, data from the skin conductivity sensors, data from the microphones and/or other voice sensors, and the like). In some implementations, when the bioindicators contradict the developmental data uploaded by a responsible person, the remote server(s) prompts the responsible person to update their evaluation. In some cases, the prompt can cause the responsible person to provide an alternative, more accurate evaluation. In other cases, the prompt can cause the responsible person to explain the apparent contradiction (e.g., to explain that the elevated heart rate is reflective of exercise during (or just prior to) the interaction, the relatively high stress levels are at least partially the result of a missed nap, and the like). In some implementations, the amount of corroboration and/or contradiction between the developmental data and the bioindicator data can be recorded and used in various methods of the system (e.g., to assign a confidence level to each evaluation that can be used in weighting the evaluations in an aggregation of developmental data).

In some implementations, the wearable device can communicate updates on the physical, mental, and/or emotional status of the supervised person throughout the day and/or the toddler’s location. To do so, the wearable device can communicate with an electronic device of any nearby responsible person (e.g., the electronic device of the childcare workers), a nearby beacon, one or more internet of things (IoT) devices in its vicinity, and/or directly to the remote server (e.g., over an internet or cellular connection). The status updates can allow the controlling person to easily monitor the health and development of the supervised person throughout the day, as well as the supervised person’s physical location. Further, the status updates can be timestamped, allowing the system to easily cross-reference bioindicator data with significant events and/or evaluations from the responsible persons.

In some implementations, the developmental data and/or the bioindicator data (sometimes referred to collectively as “target data”) are used to establish baselines for each of the supervising person(s) and/or the supervised person(s) (e.g., to establish that Child-A is generally shy around childcare providers while Child-B is very interactive with childcare providers; establish that Supervisor-1 is a generally harsh evaluator while Supervisor-2 is a generally lenient evaluator; and the like); evaluate and/or track the physical, emotional, cognitive, and/or social developmental status of the supervised person(s); predict how various changes (e.g., changes in daily routine, nutrition, academic courses, and the like) will impact the developmental status of the supervised person(s); and/or make recommendations for changes to intentionally impact the developmental status of the supervised person(s).

The target data, baselines, developmental status, predicted impacts of changes, and/or recommended changes can then be reviewed and utilized by the responsible persons. As a result, for example, the system can allow parents to make more informed decisions about the daily routines they request that childcare workers follow while responsible for their toddlers. In another example, the system can provide childcare workers with general and/or child-specific recommendations for changes in daily routines to positively impact the development the toddlers under their supervision.

In a specific, non-limiting, example, the system can include two parents, a daycare service with multiple childcare providers, and one or more toddlers equipped with wearable devices. After various interactions with the toddler (e.g., greeting the toddler, separation from parents, after a snack, after leaving the toddler alone, etc.), various relevant periods (e.g., after a quarter, semester, camp period, etc.), a detected stress event, a detected proximity, and/or at various other suitable times, the system (e.g., through the remote server) can prompt the childcare providers to provide an evaluation of each toddler. In some implementations, the evaluation is a form that includes various standardized prompts that allow the childcare provider to quickly enter the evaluation. The prompts can be related to standardized developmental goals, classifications, milestones, and the like (e.g., related to the WHO classifications, the CDC developmental milestones, and the like).

In a related example related to the care of an elderly person, various research institutions have identified links between the physical, emotional, and/or cognitive status of elderly individuals and their long-term functional capacity and/or lifespan. The prompts can be related to identifying various physical and/or cognitive events that may indicate that a medical intervention is necessary to improve the supervise person’s long-term functional capacity and/or lifespan.

The system can then upload the target data for each supervised person to the server cloud. There, one or more modules can format the target data, classify and label the target data, and/or link associated target data (sometimes referred to collectively as “processing” the target data). After the processing, one or more modules can apply an artificial intelligence and /or machine learning algorithm (referred to collectively as an “AI/ML algorithm”) to the target data. In some implementations, the AI/ML algorithm is trained on the target data from multiple supervised persons to generate a predictive model. The predictive model can be used to evaluate new target data to identify a current developmental status for a supervised person, predict the impact various changes (or lack thereof) will have on the developmental status for the supervised person, and/or generate recommendations for changes to intentionally impact the developmental status for the supervised person in a desired way.

Returning to the specific example above, the AI/ML algorithm can train a predictive model on the target data for multiple toddlers over time. Once the predictive model is trained, the system can apply the predictive model to any new target data for a specific toddler to identify their current developmental status. The system can then make the current developmental status available to the parents and/or the childcare providers, allowing the responsible persons to monitor and/or track the development of the toddler. Additionally, or alternatively, the system can apply the predictive model to the new target data and identified changes (e.g., changes in daily activities, changes in daily nutrition, changes in supervision, an indication of no changes, and the like) to predict how the changes (or lack thereof) will impact the toddler’s developmental status. Additionally, or alternatively, the system can apply the predictive model to the new target data to generate recommendations for changes to intentionally impact the toddler’s developmental status in an indicated manner. For example, after reviewing the developmental status, the parents can indicate that they would like to accelerate (or otherwise change) their toddler’s cognitive development, and the predictive model can identify changes/suggestions/recommendations for doing so.

In some implementations, the predictive model is generally applicable to supervised persons, thereby identifying broad trends between developmental statuses and daily life. In such implementations, as the system accrues larger databases of target data, the predictive model is expected to identify previously unknown correlations and possible causal relationships between daily activities, nutrition, supervision, etc. and the development of supervised persons. In some implementations, the predictive model is specific to the supervised person and/or includes factors that are specific to the supervised person. For example, the predictive model can include variables that adjust the predictive model to a specific supervised person to account for variations in how daily life and/or changes to daily life impact the supervised person. In a specific, non-limiting example, the predictive model can include a variable that accounts for how quick a specific toddler responds to additional physical activity to adjust predictions of the impact of a change to include more physical activity in their daily routine.

In some implementations, the system can make the collected target data available to one or more research institutions. Because the system is able to non-invasively collect a large amount of target data, the system is expected to significantly expand the possibilities of research into the target data and/or significantly improve the results of any such research. In some implementations, the system can receive predictive models back from the research institution(s) (e.g., resulting from research on the target data provided to the research institution(s)) and utilize the predictive models in the manner discussed above. In some implementations, the system can make a predictive model generated by the AI/ML algorithms available to the research institution(s) to prompt further study. Purely by way of example, where the system identifies a new causal relationship (e.g., based on a newly identified correlation), the system can provide a predictive model with the correlation to the research institution(s), allowing the research institution(s) to find (or disprove) a causal relationship and/or otherwise explain the correlation.

For ease of reference, components of the system are sometimes described herein with reference to top and bottom, upper and lower, upwards and downwards, and/or horizontal plane, x-y plane, vertical, or z-direction relative to the spatial orientation of the implementations shown in the figures. It is to be understood, however, that the components can be moved to, and used in, different spatial orientations without changing the structure and/or function of the disclosed implementations of the present technology.

Further, although primarily discussed herein as a system for use to supervise a toddler, one of skill in the art will understand that the scope of the technology is not so limited. For example, the systems and methods disclosed herein can also be used to aid and/or supplement the supervision of a baby, a child, an elderly person, a person with a mental disability, and/or any other person that requires at least partial supervision over their daily activities. Accordingly, the scope of the technology is not confined to any subset of implementations described herein.

### Systems of the Present Technology

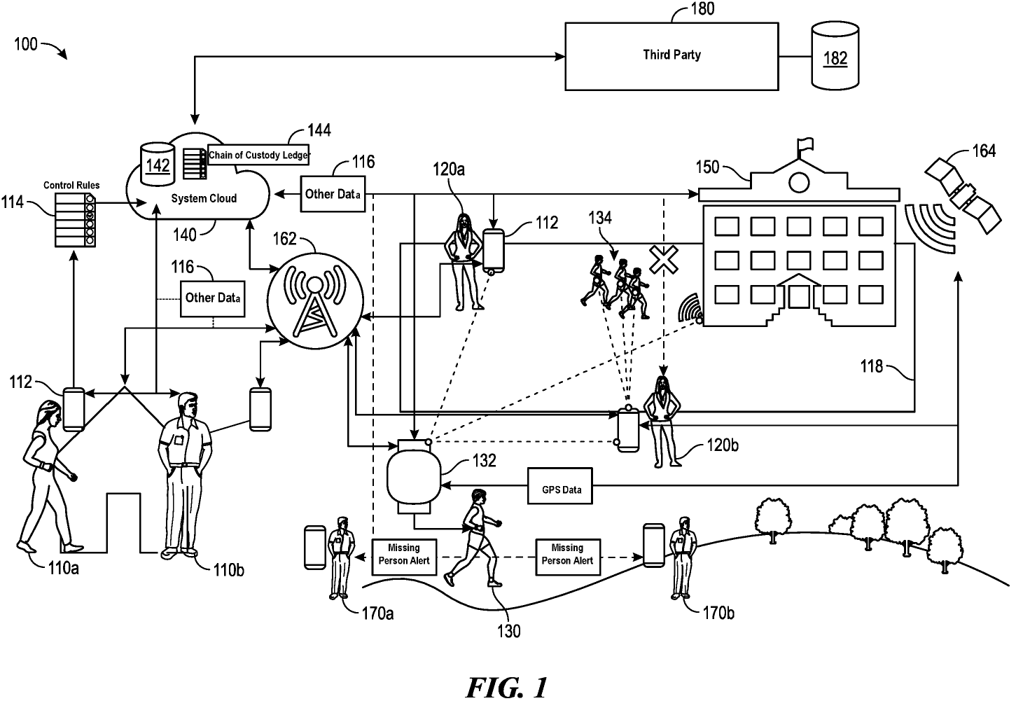

FIG. 1 is a schematic view of a system 100 configured in accordance with some implementations of the present technology. The system 100 can be used for monitoring supervised persons 130, scoring supervising persons 120, and tracking the development of the supervised persons 130. For example, the system 100 interconnects one or more controlling persons 110, one or more supervising persons 120, and one or more supervised persons 130 (e.g., a baby, toddler, child, elderly person, differently abled person, and/or any other person requiring supervision) with a remote server 140 to share information related to the health and development of the supervised person 130.

For simplicity, the illustrated implementation includes two controlling persons 110 (referred to individually as a “first controlling person 110a” and a “second controlling person 110b”). In various implementations, the first and second controlling persons 110a, 110b can be a first parent, godparent, grandparent, siblings, any legal guardian, an adult providing care to an elderly family member, an adult providing care to another elderly person, and/or any other suitable person that exercises a degree of control over the supervised person’s daily activities and/or overall well-being. Further, in various implementations, the system 100 can include fewer, or additional, controlling persons 110. For example, the system 100 can include two parents and an older sibling; a single parent; or multiple siblings that share caregiving responsibilities for an elderly parent.

The illustrated implementation also includes one or more supervising persons 120 (two shown, referred to individually as a “first supervising person 120a” and a “second supervising person 120b”). In various implementations, the first and second supervising persons 120a, 120b can be various caregivers (e.g., a child care worker (such as a daycare worker, nursery worker, nanny, or babysitter), a family caregiver, a home health caregiver, an assisted living nurse or any other nursing practitioner, and the like), a preschool or elementary school teacher or other school officials, and/or any other suitable person responsible for the supervised person 130. Further, the supervising person(s) 120 can also be responsible for one or more additional supervised persons For example, when the supervising persons 120 are a part of a daycare provider, the additional supervised persons 134 can be the other toddlers and/or children entrusted to the daycare.

In the illustrated implementation, each of the controlling persons 110 and the supervising persons 120 (e.g., the responsible persons) are interconnected to a remote server 140 (e.g., a cloud server or other suitable server) in the system 100 through one or more electronic devices 112 (e.g., a smartphone, tablet, personal computer, personal assistant, wearable device, IoT device, AR/VR device, and the like). For example, each of the electronic devices 112 can communicate via shortrange wireless components (e.g., Bluetooth® components and the like), internet communication components that connect to a wireless or wired network (e.g., the internet), and/or cellular components that connect to a cellular network 162. The communication can help the responsible persons maintain a record of the control over of the supervised person 130 within the system; coordinate regarding required activities for the supervised person 130; coordinate regarding the developmental status of the supervised person 130; and/or communicate any other suitable information. The actions of the responsible persons in the system 100 can be performed through one or more user interfaces and/or modules on the electronic devices 112. Accordingly, one of skill in the art will understand that, as used herein, the actions of the responsible persons with respect to the system 100 can be performed through the electronic devices 112 (e.g., through various subsystems 300, 400 thereon, discussed in more detail below) and therefore refer to the modules and/or functionality of the electronic devices 112, unless otherwise indicated.

Purely by way of example, the controlling persons 110 can upload, edit, and/or update a set of control rules 114 that are stored within the remote server 140, and the control rules 114 can then be accessed by a supervising person 120 to ensure appropriate supervision of the supervised person 130. In various implementations, the control rules 114 include rules for how much supervision must be given to the supervised person 130 (e.g., constant supervision, whether semi-supervised playtime is allowed, and the like); who is permitted to supervise the supervised person 130 (e.g., when supervision must be maintained by a particular caregiver or set of caregivers rather than handed off); what activities the supervised person 130 can engage in such as types of play, field trips, sports, movie and television watching controls, and the like; what activities the supervised person 130 must engage in such as a daily nap, daily exercise, learning activities, and the like; a preferred and/or required schedule and/or routine for the supervised person 130; what foods the supervised person 130 can consume and/or cannot consume; a predefined geographic location for the supervision of the supervised person 130; geographical limits to field trips, errands, and/or any other deviations from where the supervised person is dropped off; rules for proscribed medical care, such as a preferred hospital, pediatrician, authorized medications (e.g., Advil®, Tylenol®, and the like and/or authorized dosages of medications; authorized visitors for the supervised person 130; authorized persons to receive the supervised person 130 (e.g., a parent can specific that a grandparent, godparent, and/or older sibling can pick up their child from daycare); and/or various other suitable rules related to the supervision of the supervised person 130. In some implementations, the system 100 includes tiers of control between the first and second controlling persons 110a, 110b. For example, the first controlling person 110a can have more power in setting and/or adjusting the control rules 114 than the second controlling person 110b (e.g., allowing parents to exercise more control than an older sibling).

In another example, as further illustrated in FIG. 1, each of the responsible persons can upload, edit, update, and/or review other data 116 data related to the supervised person 130 and/or interactions with the supervised person 130. For example, each of the responsible persons can upload evaluations of their interactions with the supervised person 130 (e.g., during the schedule mandated by the control rules 114) that allow the system 100 to assess the physical, emotional, cognitive, and/or social development of the supervised person 130. The evaluations can be completed using a form response that includes various objective evaluations (e.g., whether a child made eye contact when saying hello; whether the child remembers the name of the responsible person; whether the child is able to walk and/or run on their own; and the like) and/or various subjective evaluations (e.g., rating, on a scale, the child’s comfort being away from a parent or guardian, being in a new environment, meeting new people, and the like; evaluating the child’s mood from one or more selectable options; and the like). In another example, each of the responsible persons can upload a report whenever a supervised person 130 achieves a developmental milestone (e.g., when a toddler is able to read for the first time). In some implementations, each of the responsible persons has a platform (e.g., through their electronic devices 112) to upload, edit, and/or review reports on events. For example, when the supervised person 130 has a bad fall (or other stressful experience), the responsible person caring for the supervised person 130 can upload a report of the fall (or other experience) for review by the other responsible person(s) and/or for review by various modules in the system 100. In some implementations, the other data 116 can be supplemented and/or contradicted by reports from a wearable device 132 on the supervised person 130.

In some implementations, the controlling persons 110 have a platform to upload information related to the supervision and developmental status of the supervised person 130, such as known allergies, known medical conditions, medical history information, known behavioral patterns, recent developments or updates, known mental impairments, and/or various other data that impacts the development of the supervised person 130. Non-limiting examples of medical history information can include information on vaccinations, family medical history, diagnoses specific to the supervised person, past medical events such as surgeries, illnesses, and/or major medical events (e.g., seizures). Non limiting examples of recent developments or updates include recent diagnoses, broken bones and/or other physical trauma, recently experienced mental and/or emotional trauma such as the loss of a family member, cognitive and/or behavioral developments such as learning to use the restroom for toddlers and loss of memory in adults, and the like.

In some implementations, each of the responsible persons can upload, edit, update, and/or review developmental data related to daily activities of the supervised person 130. For example, each of the responsible persons can upload data related to the daily activities of the supervised person 130 such as reports on physical and/or cognitive exercise, estimated sleep during naps and/or overnight, nutritional intakes, eating and/or sleeping patterns (e.g., when the supervised person 130 tends to nap and/or be hungry), an assessment of various cognitive elements (e.g., ability to learn, participate in class, attention span, memory, comprehension of text, and the like), a mood of supervised person, reports on injuries and/or stress events, reports on medication administrated to the supervised person, and/or any other suitable information about the daily activities of the supervised person 130.

Additionally, or alternatively, as the responsible persons complete actions and/or communicate through the system 100, their actions and/or communications can be routed through and/or relayed to the remote server 140. Further, as illustrated in FIG. 1, the remote server 140 includes one or more databases 142 (one shown) that can store a record of the control rules 114, various communications between the responsible persons, developmental data contained in communications, other data related to the supervised person 130 (e.g., medical history data, allergies, background data, food preferences and/or permissions, supervisory care instructions, and the like), a record of the control over the supervised person 130 (e.g., responsibility for providing care to and/or supervising the supervised person 130), and/or any other suitable information. Further, in some implementations, the remote server 140 maintains a secure ledger with any of the information discussed above. For example, in the illustrated implementation, a record of any handoffs can be recorded in a chain of custody ledger 144 (“ledger 144”) that is stored on and accessed through the remote server 140. The ledger 144 can maintain a complete record of who was responsible for the supervised person 130 throughout a day in a secure, unalterable manner. Accordingly, for example, the ledger 144 allows a parent to review the supervision of their toddler when the toddler indicates that they had a particularly good or bad day.

As further illustrated in FIG. 1, the responsible persons can communicate with the remote server 140 to create, edit, receive, and/or download the other data 116. For example, in addition to the metrics and evaluations discussed above, the other data 116 communicated to the remote server 140 include pictures/videos/audios of the supervised person 130, reports on events related to the supervised person 130, reviews of supervising persons 120, other information related to the supervision of the supervised person 130, indications of deviations from a daily routine (e.g., that the supervised person 130 missed a nap or had some additional meal), and/or other information related to the daily activities of the supervised person 130.

In the illustrated implementation, the wearable device 132 can communicate data to the electronic devices 112 and/or directly the remote server 140 through any suitable network connection. To do so, as described in more detail below, the wearable device 132 can include a shortrange wireless communication component, an internet communication component, and/or a cellular component. Further, to collect the data, the wearable device 132 can include one or more sensors that collect bioindicator data that helps monitor the health and mental status of the supervised person 130, such as skin temperature sensors, photoplethysmogram (PPG) sensors, accelerometers, skin conductivity sensors, heart-rate variability sensors, resting heart rate sensors, sweat chemical composition sensors, nervous system electrical sensors, air quality sensors, UV exposure sensors, sensors to detect environmental chemicals, blood oxygen and/or pulse oxygen sensors, voice recognition, electrocardiogram (ECG) sensor, pressure sensors, gyroscopes, magnetometers, and the like. The bioindicator data from the sensors can be continuously and/or periodically communicated throughout the system 100. For example, the wearable device 132 can communicate updates to the electronic device 112 of any responsible person whenever their electronic devices 112 are within range of the shortrange wireless communication component; to the remote server 140 through the internet when a wireless connection is available; and/or to the remote server 140 through the cellular network 162 whenever the shortrange and internet options are unavailable. Additional details on examples of suitable wearable devices, and associated systems and methods, are disclosed in U.S. Provisional Pat. Application No. 63/260,440 by Monica Plath, filed Aug. 19, 2021; U.S. Provisional Pat. Application No. 63/247,692 by Monica Plath, filed Sep. 23, 2021; and U.S. Pat. Application No. 17/891,781 by Monica Plath, filed Aug. 19, 2022, the disclosures of each of which are incorporated herein in their entireties by reference.

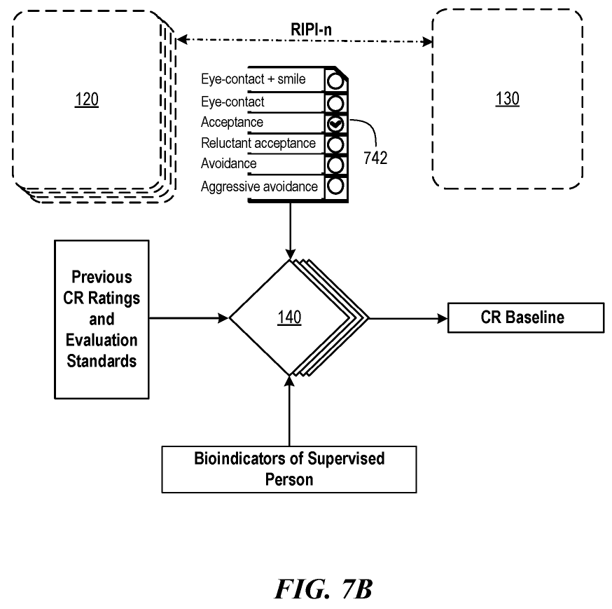

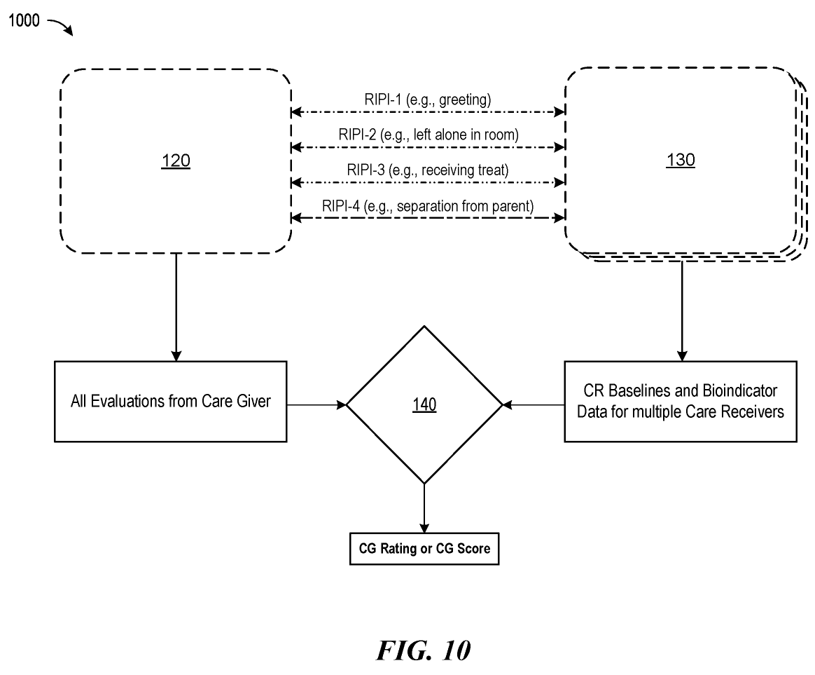

The bioindicator data from the sensors can then be relayed to the remote server 140 to monitor and/or track the physical, emotional, and/or mental condition of the supervised person 130. Purely by way of example, the bioindicator data from the sensors can be processed and/or mined by one or more modules on the remote server 140 to more accurately evaluate a current monitoring the physical, emotional, and/or mental condition of the supervised person 130; track the physical, emotional, and/or mental condition of the supervised person 130 over time; and/or evaluate the impact of various responsible persons on the physical, emotional, and/or mental condition of the supervised person 130. The data from the sensors can also be mined by one or more modules on the remote server 140 to monitor the physical, emotional, social, and/or cognitive status of the supervised person 130 and/or to supplement, support, and/or contradict reports from the responsible persons. Additional details on how the remote server 140 can use the bioindicators to supplement, support, and/or contradict reports from the responsible persons are discussed below with respect to FIGS. 8 and 11.

As the responsible persons complete actions (e.g., complete evaluations) and/or communicate through the system 100, their actions and/or communications can be routed through and/or stored in one or more databases 142 (one shown) at the remote server 140. In some implementations, as discussed in more detail below, the remote server 140 includes one or more modules that use the evaluations and other data to establish personality baselines for the supervised person 130; evaluate the development of the supervised person 130; establish various baselines for each of the responsible persons, evaluate and rate the supervising person 120; make recommendations to the controlling person 110 related to the physical, emotional, cognitive, and/or social development of the supervised person 130; and/or various other suitable functions.

For example, the remote server 140 can use the developmental data and the bioindicator data (referred to collectively as “target data”) to generate one or more predictive models specific to a particular supervised person 130 and/or applicable to supervised persons overall. The predictive models can be used to evaluate target data identify a current physical, emotional, cognitive, and/or social developmental status (also referred to collectively herein as the “developmental status”) for a supervised person, predict the impact various changes will have on the developmental status for the supervised person, and/or generate recommendations for changes to intentionally impact the developmental status for the supervised person in a desired way.

The specific predictive models can also be customized to a specific supervised person, for example, by accounting for their typical reactions to changes (e.g., whether they are more or less sensitive to changes) to more accurately identify how activities and/or actions will impact the developmental status of the particular supervised person. The general predictive models can be used to help identify broad trends in the impact of various activities and/or actions on the developmental status of supervised persons (e.g., to assess how various levels of exercise generally impact the developmental status of toddlers), to identify previously unrecognized correlations that may indicate some causal relation, and/or to make broad recommendations for activities and/or actions to intentionally impact the developmental status of supervised persons. Further, the general predictive models can be used to assess the developmental status of a new supervised person in the system given a small amount of target data on the new supervised person.

In some implementations, the wearable device 132 can process the bioindicator data, before sending the update related to the bioindicator data, to better monitor the physical, emotional, and/or mental condition of the supervised person 130. For example, the wearable device 132 can process the bioindicator data to detect emotional, cognitive, and/or physical developments and/or events (e.g., a high-stress event) and send the update in response to the detected event.

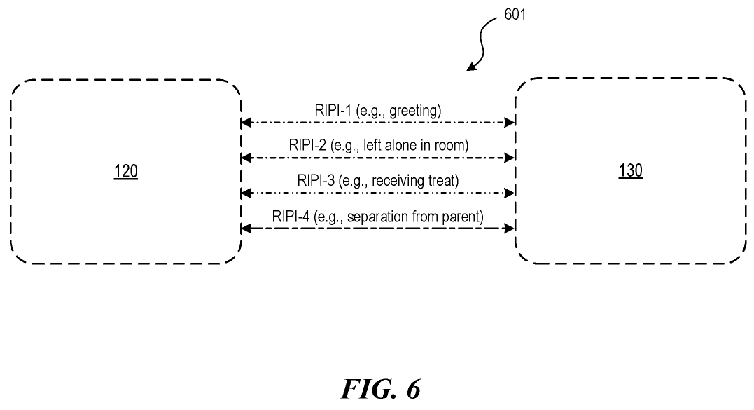

The wearable device 132 can also include a global positioning system (GPS) component that communicates with one or more GPS satellites 164 to track the location and/or movement of the supervised person 130. The GPS component allows one or more responsible persons to define a geofence boundary 118 to aid in monitoring the supervised person 130. For example, the geofence boundary 118 can surround the perimeter of a playground associated with a daycare or school. If the supervised person 130 exits the geofence boundary 118 without a suitable explanation, the system 100 can send an alert to any of the responsible persons. Additional details on the geofencing aspects of the system 100 are discussed below, especially in reference to FIG. 6.

In some implementations, one or more of the additional supervised persons 134 are also wearing a wearable device 132, connecting them to the supervising persons 120 and their own respective controlling person(s) (not shown). In some implementations, the geofencing features discussed above can be implemented broadly for each of the supervised persons 130, 134. For example, the supervising persons 120 can define the geofence boundary 118 as broadly applying to each of the supervised persons 130, 134 at a single time (e.g., while children are at recess), such that if any of the supervised persons 130, 134 break the geofence boundary the supervising persons 120 are alerted. Similarly, in some implementations, one or more control rules 114 are be set for each of the supervised persons 130, 134 broadly.

In the implementation illustrated in FIG. 1, the supervising persons 120 are members of an associated caregiving facility 150, such as a contracting school, daycare or other childcare facilities, assisted living facility, nursing center, and/or any other suitable entity. In some implementations, the caregiving facility 150 includes features that assist communication throughout the system 100 (e.g., various IoT-enabled devices, beacons, WiFi hotspots, sensors, and the like) dispersed throughout the caregiving facility 150. Further, in some implementations, the caregiving facility 150 includes a set of supervising persons 120 that have an organizational hierarchy and/or sub-divisions for supervising groups of one or more supervised persons 130.

For example, the first supervising person 120a can define control rules 114 applying to the supervised persons 130, 134 that the second supervising person 120b can review and follow. In some implementations, the control rules 114 defined by the first supervising person 120a must be within a tolerance of the control rules 114 defined for each of the supervised persons 130, 134 individually (e.g., by their respective controlling persons). To provide a specific example, the controlling persons of each of the supervised persons 130, 134 may indicate that the supervised persons 130, 134 are not to leave a specific facility (e.g., a daycare facility) without their permission. In turn, the first supervising person 120a can indicate that the second supervising person 120b cannot remove the supervised persons 130, 134 from a specific room within the facility without the permission of the first supervising person 120a.

As further illustrated in FIG. 1, the system 100 can also connect with one or more third parties 180 (one shown), each having its own database 182 to store information related to the developmental status of the supervised person 130 and/or the additional supervised persons 134. In some implementations, the remote server 140 can share some, or all, of the target data with the third parties 180. The third parties 180 can then study the target data to, for example, identify general trends in development for supervised persons based on their experiences, bioindicators, and/or daily routines. Additionally, or alternatively, the third parties 180 can mine the target data to generate predictive models (both specific and general) for supervised persons. Once generated, the third parties 180 can communicate the predictive models back to the remote server 140, which can then use the predictive models in one or more modules accessible by the responsible persons. Like the predictive models generated by the remote server 140, the predictive models generated by the third parties 180 can be used to identify a current developmental status of the supervised person 130 (or any of the additional supervised persons 134); predict how various changes will impact the developmental status of the supervised person 130; and/or make recommendations for changes to intentionally impact the developmental status of the supervised person 130.

By collecting and linking bioindicator data with data from the supervised persons, as well as collecting the target data in bulk, the system 100 is expected to greatly improve the accuracy of predictive models used to assess the current developmental status of supervised persons and make decisions about changes to the supervised persons' lives. For example, by closely monitoring the bioindicators of a child alongside assessments of the child, the system 100 is expected to generate more accurate predictive models for physical, emotional, cognitive, and/or social development in children. In another example, by closely monitoring the bioindicators of an elderly person alongside assessments of the elderly person, the system 100 is expected to generate more accurate predictive models that allow for early detection of various illnesses that cause a decline and/or early intervention against the illnesses. Further, the system 100 is expected to provide a non-invasive point of entry for academic research into human development, together with checks that help ensure the data from the system is accurate (e.g., checks between evaluations of the supervised person and their bioindicator data).

As further illustrated in FIG. 1, the system 100 can also include a connection to one or more additional persons 170 (two shown, labeled 170a and 170b). The additional persons 170 can be alerted by a variety of functions in the system to search for the supervised person 130, check on the supervised person 130, rescue the supervised person 130, and the like. Purely by way of example, when the supervised person 130 breaches a geofence boundary, the responsible persons can be alerted to the breach. Any of the responsible persons can then instruct the system 100 to notify additional persons 170 that the supervised person 130 needs to be located. In various specific, non-limiting examples, the additional persons 170 can be emergency responders (e.g., security officers, police officers, fire departments, neighborhood watch, and the like), other parents or guardians on the system 100, other supervising persons on the system 100, and the like. The notification of the additional persons 170 can help locate the supervising person 130 quickly to resolve the breach. In another example, when the supervised person 130 presses a panic button on the wearable device 132, the system 100 can respond by alerting the additional persons 170 to check on and/or rescue the supervised person 130. In yet another example, when the bioindicator data for a supervised person 130 indicate prolonged and/or recurring periods of stress, the system 100 can alert additional persons 170 (e.g., child services) to check on the supervised person 130.

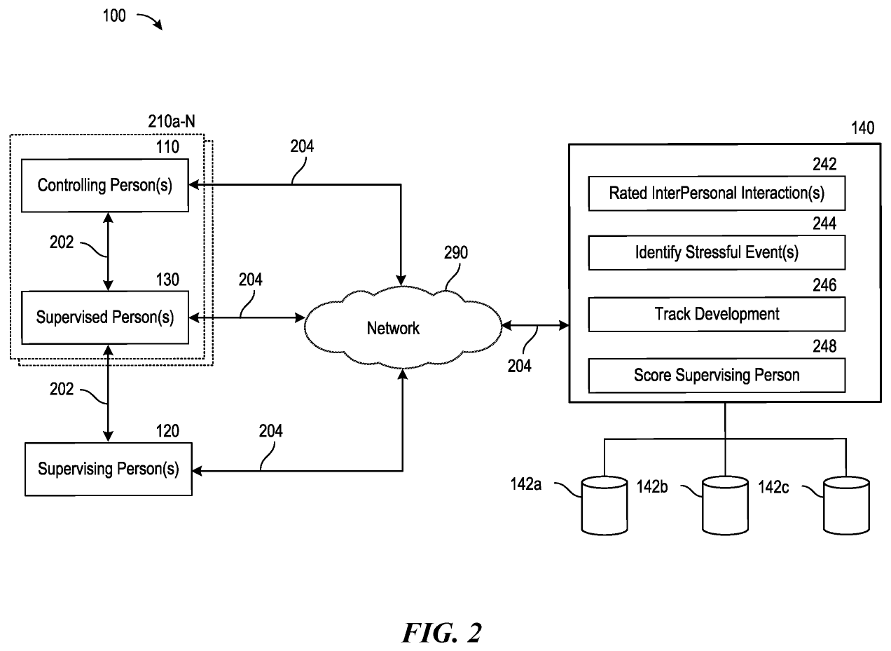

FIG. 2 is a network diagram of the system 100 for scoring supervising persons 120 and tracking the development of supervised persons 130 in accordance with some implementations of the present technology. As illustrated in FIG. 2, supervised person 130 can have a shortrange communication channels 202 with each of the controlling person(s) 110 and the supervising person(s) 120. As discussed above, the shortrange communication channels 202 can be established over any suitable short-range wireless standard (e.g., Bluetooth®, Zigbee®, Z-Wave®, Wi-Fi HaLow®, or any other suitable short-range standard). The shortrange communication channels 202 allow the supervised person 130 to communicate locally with the controlling person(s) 110 and the supervising person(s) 120 via a relatively low-energy, secure standard. However, the supervised person 130 is not always within range of one of the controlling persons 110 and the supervising persons 120 to establish the shortrange communication channels 202. Accordingly, as further illustrated in FIG. 2, each of the controlling persons 110, the supervising persons 120, and the supervised person 130 can also communicate with a network 290 (e.g., an internet network, a cellular network, and so on) via a network communication channel 204 (e.g., a WNIC connecting to WiFi and/or a cellular communication channel).

In the illustrated implementation, the controlling person(s) 110 and the supervised person(s) 130 are grouped as a client 210 linked to the supervising person(s) 120. As further illustrated in FIG. 2, the system can include any number of clients 210 (N number shown as clients 210a-N) linked to the supervising person(s) 120. For example, the system 100 can include one client, two clients, three clients, five clients, ten clients, one hundred clients, or any other suitable number of clients linked to the supervising person(s) 120. Each client 210a-N can represent one data source that the system 100 uses in rating and/or evaluating the supervised person(s) 120.

As further illustrated in FIG. 2, the network 290 is connected to the remote server 140 through a network communication channel 204. Accordingly, the remote server 140 can communicate with each of the controlling person(s) 110, the supervising person(s) 120, and the supervised person 130 through the network 290, for example to receive data and/or ratings related to interpersonal interactions, data related to the development of the supervised person 130, and/or data related to evaluations of the supervising person(s) 120. In the illustrated implementation, the remote server 140 includes three databases 142a-142c to store the data and/or any relevant communications. The remote server 140 can then use the data to execute various functions related to monitoring the development of the supervised person 130 and/or rating the supervising person(s) 120. For example, in the illustrated implementation, the remote server 140 includes five modules (referred to individually as first-fifth modules 242-250) that can be stored in the databases 142a-142c and executed in response to various activities in the system 100 (e.g., in response to a request from a controlling person 110).

In the first module 242, the remote server 140 processes data on rated interpersonal interactions (e.g., RIPI data). The RIPI data can include evaluations from one or more responsible persons, including objective and/or subjective evaluations of interactions with the supervised person; evaluations from the controlled person of the supervising person, including objective and/or subjective evaluations of the interactions between the supervising person and the supervised person; and/or bioindicator data from the wearable device 132 (FIG. 1) on the supervised person 130. When processing the RIPI data, the remote server 140 can generate a rating of each of the parties to an interpersonal interaction (e.g., a rating for the controlling person 110 and the supervised person 130, or a rating for the supervising person 120 and the supervised person 130).

The remote server 140 can then use the ratings to set and/or update an interaction baseline for the supervised person 130 (referred to herein as a “CR baseline”) representing baseline of their typical interaction and/or their development level. The CR baseline for the supervised person 130 can influence future ratings based on expected interactions with the supervised person 130 (e.g., an outgoing interaction with a shy toddler can be rated higher and/or a reserved interaction with an outgoing toddler can be rated lower). Similarly, the remote server 140 can use the ratings to set and/or update an interaction baseline for the supervising person 120 (referred to herein as a “CG baseline”), and the CG baseline can influence future ratings based on expected interactions with the supervising person 120. Additionally, or alternatively, the remote server 140 can use the RIPI data to set an evaluation baseline (referred to herein as an “E baseline”) for how a responsible person (e.g., either the controlling person 110 and/or the supervising person 120) typically evaluates interactions. For example, the E baseline can account for particularly harsh and/or lenient evaluators when generating ratings of the parties to an interaction. Once the E, CG, and/or CR baselines have been set, the remote server 140 can use the E, CG, and/or CR baselines in other modules of the remote server 140 to rate the persons in following interactions, evaluate and/or track the developmental status of the supervised person 130 and/or to generate a RIPI score for the evaluator.

In the second module 244, the remote server 140 identifies stressful events. In some implementations, the remote server 140 identifies the stressful events from the bioindicator data from the wearable device 132 (FIG. 1). For example, a skin conductivity sensor can be used to identify when the supervised person 130 experiences an increase in stress. In turn, the increase in stress can signify a stressful event, especially when, for example, the increase in stress is accompanied by other changes in bioindicators (e.g., an increase in heart rate). In some implementations, once the remote server 140 identifies the stressful events, the remote server 140 prompts the one or more of the responsible persons for an evaluation of the supervised person and/or to account for the stressful event. Purely by way of example, the remote server 140 can prompt a daycare provider to evaluate how a child behaved during and/or after a stressful event, such as an introduction into a new environment for the child. In some implementations, the remote server 140 then uses the evaluations to update the CR baseline for the supervised person 130 and/or evaluate the developmental status of the supervised person 130. In some implementations, the remote server 140 uses the frequency of stressful events experienced by a supervised person 130 under the control of a supervising person 120 to update the CG baseline for the supervising person 120 and/or to update the RIPI score for the supervising person 120. In a specific example, when a child frequently experiences stressful events with a particular childcare provider, the frequent stresses can negatively impact the RIPI score for the childcare provider. In some implementations, the remote server 140 uses the content of the evaluations to update the CG baseline for the supervising person 120 and/or to update the RIPI score for the supervising person 120.

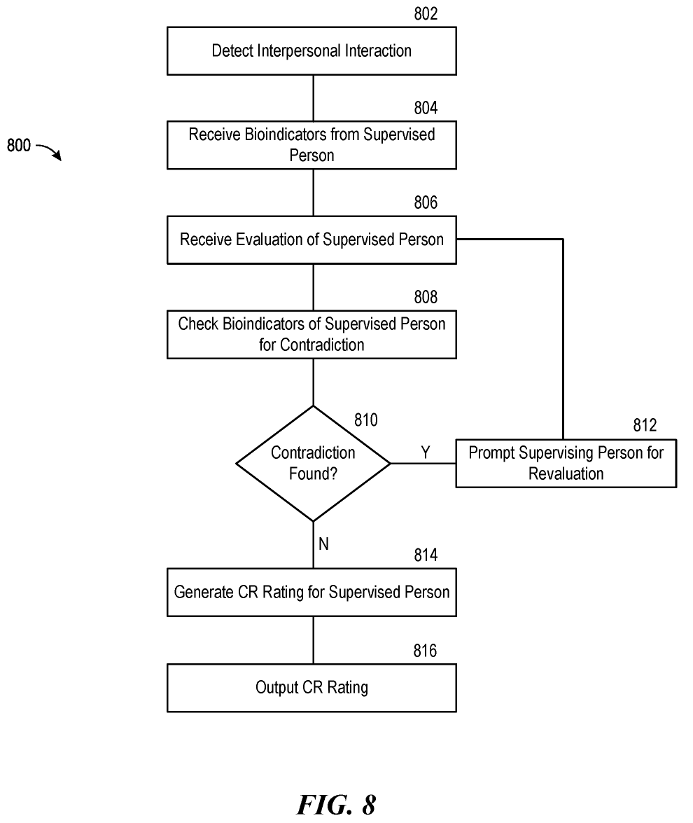

In the third module 246, the remote server 140 evaluates and tracks the development of the supervised person 130 over time. The remote server 140 can use RIPI data from a recent evaluation and/or bioindicator data, along with a relevant CR baseline, to evaluate the developmental status of the supervised person 130. Purely by way of example, the remote server 140 can use a CR baseline indicating that a child is generally insecure in new environments with and evaluation and/or bioindicator data showing they were comfortable in a new environment to determine that the child is developing more security and confidence in new environments. In another example, the evaluation data can include objective indications of development (e.g., that a child achieved a relevant milestone such as walking, speaking, reading, using the restroom alone, or any other suitable milestone). Additional details on determining the current development of the supervised person 130 are discussed below with respect to FIGS. 8 and 9.

Once the remote server 140 identifies a current developmental status of the supervised person 130, the remote server 140 can track the development of the supervised person 130 over time. Tracking the development of the supervised person 130 over time can include recording each developmental status output by the first module 242, comparing developmental statuses to identify changes and/or identify trends in the developmental status, comparing one or more developmental statuses to an expected status for the supervised person 130 (e.g., checking whether the supervised person 130 has achieved expected developmental milestones for their age), and/or predicting future changes based on identified trends. In some implementations, in the third module 246, the remote server 140 makes recommendations to one or more of the responsible persons based on the tracked development. For example, when the remote server 140 determines that the supervised person 130 is behind developmental goals (e.g., has not achieved developmental milestones for their age), the remote server 140 can recommend interventions to the responsible persons. In a specific, non-limiting example, the remote server 140 can identify that a child is behind on cognitive development and recommend one or more cognitive exercises to the child’s parents and/or childcare providers to help improve the child’s development.

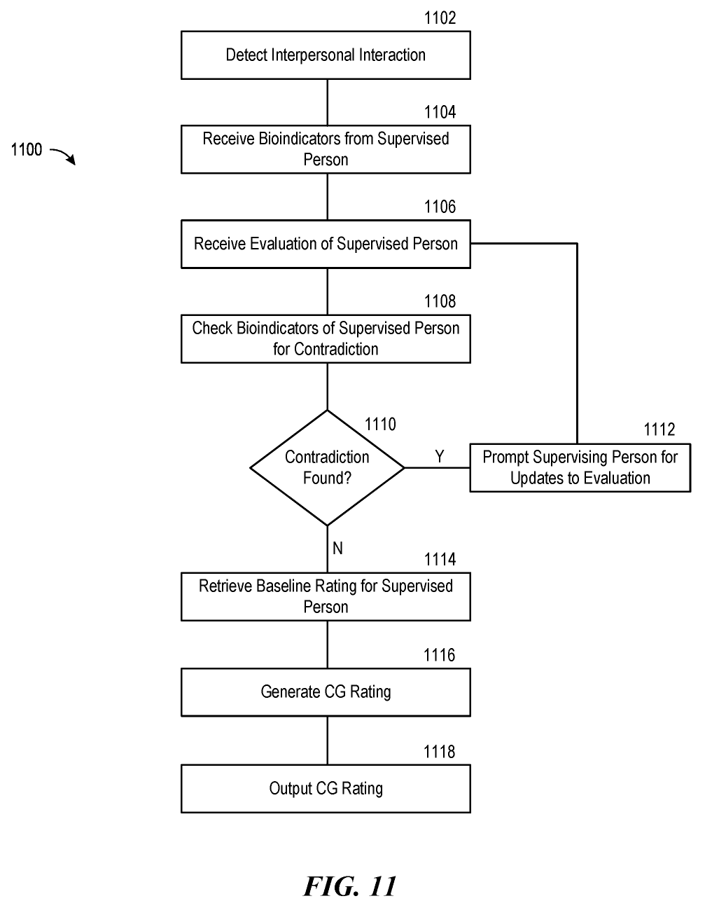

In the fourth module 248, the remote server 140 generates, updates, and/or shares a RIPI score for the supervising person 120. The remote server 140 can generate the RIPI score using the CG baseline, average interaction ratings over time, data from the evaluations the supervising person 120 provides to the remote server 140, evaluations from the controlling person(s) 110, and/or any other relevant data. The RIPI score provides an indication of the impact the supervising person 120 has on the persons under their supervision. For example, the RIPI score can indicate that a childcare provider has a positive impact on the development of children under their supervision when the RIPI data associated with the childcare provider consistently indicates improvements over time and/or accurate above average evaluations. In some implementations, the RIPI score includes various components indicating the impact the supervising person 120 has on the persons under their supervision in various areas. Purely by way of example, the RIPI score can have components reflecting the impact of the supervising person on the physical, emotional, and cognitive development the supervising person 120 has on the persons under their supervision. The components can allow the remote server 140 to identify the strong suits of a supervising person 120 as well as their weaknesses. Purely by way of example, a childcare provider may have a strong positive impact on the emotional development of a child, a neutral impact on the cognitive development of the child, and a negative effect on the physical development of the child. Additional details on generating the RIPI score for the supervising person 120 are discussed below with respect to FIGS. 11 and 12.

In some implementations, the remote server 140 provides the generated RIPI score to the supervising person 120 along with any relevant breakdown of their score. In such implementations, the remote server 140 enables the supervising person 120 to identify areas they need to focus on in providing care and supervision. Further, in some implementations, the remote server 140 recommends training exercises, readings, and/or activities the supervising person 120 can try to improve their impact on the persons under their supervision.

In some implementations, the remote server 140 makes the RIPI score available to the controlling person(s) 110 in each of the clients 210a-N. The availability of the RIPI score can allow the controlling person(s) 110 to make more informed decisions about the supervising person(s) 120 they entrust with the responsibility over the supervised person 130. Alternatively, or additionally, the availability of the RIPI score can allow the controlling person(s) 110 to more closely monitor the development of the supervised person 130 in areas that the RIPI score identifies as weaknesses for the supervising person 120.

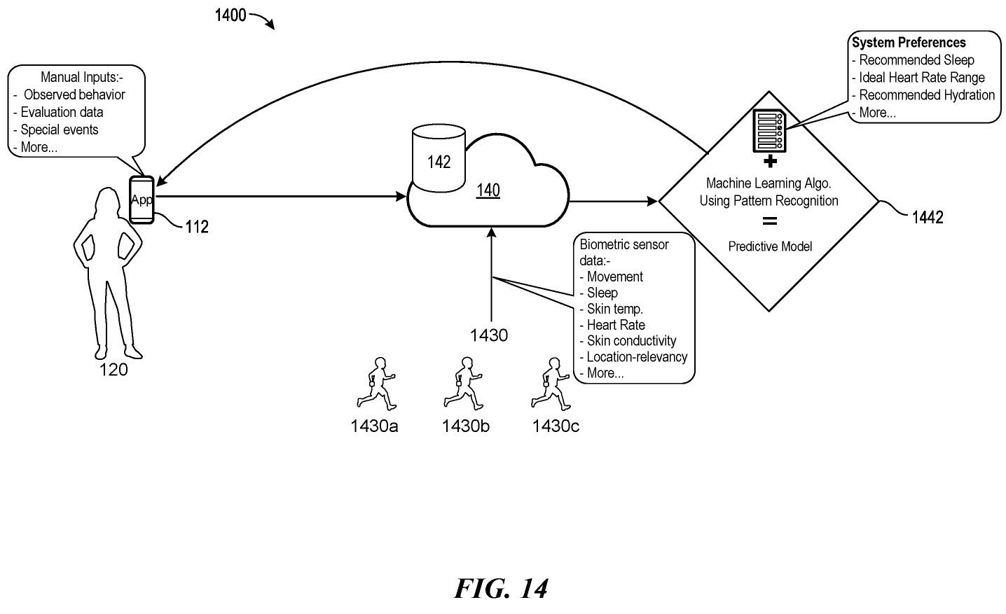

In various implementations, the remote server can include one or more additional (or alternative modules). Purely by way of example, the remote server 140 can include an additional module to processes incoming target data to format, classify and label, and/or link associated aspects of the target data (e.g., associated developmental and bioindicator data based on a relevant time of the data, the type of evaluation (e.g., stress levels, or data related to exercise) and the like). Additional details on this aspect of the first module are discussed below with respect to FIG. 15. In some implementations, processing incoming target data includes randomly selecting incoming data to be used as training data, validation data, and/or test data for use in an AI/ML algorithm. In some implementations, processing incoming target data includes linking incoming target data with data already stored on the databases 142a-142c. For example, incoming target data related to a specific supervised person can be linked to other data (e.g., previous uploads of target data, previous developmental statuses, other data uploaded by the responsible persons, and/or any other suitable data) related to the specific supervised person. In another example, incoming target data containing data of a specific type (e.g., exercise-related data, data related to specific activities (e.g., teaching interventions), and the like) can be linked to the data of that type already stored on the databases 142a-142c for other supervised persons.

In some implementations, the remote server 140 can check the assessments in the developmental data against the bioindicator data for contradictions (or corroborations) while processing the target data, and prompt responsible persons for explanations and/or reevaluations if any contradictions are found. Similarly, in some implementations, the remote server 140 can check the assessments in the developmental data from multiple responsible persons for contradictions (or corroborations) between the two assessments while processing the target data, and prompt one or more of the responsible persons for explanations and/or reevaluations if any contradictions are found. Additional details on the process of checking incoming data for contradictions and/or corroborations are discussed below.

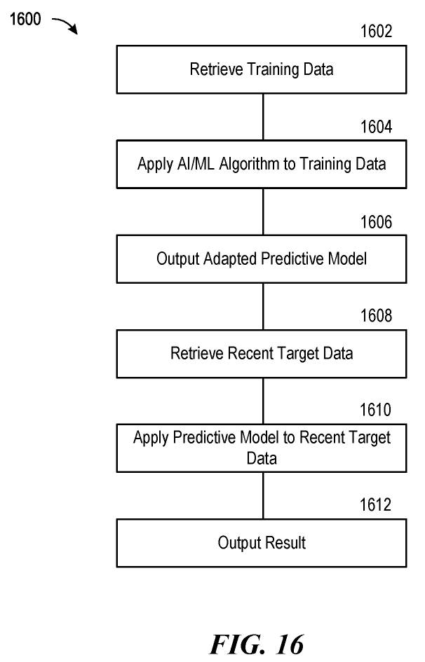

In another example, the remote server 140 can include an additional module to apply one or more AI/ML algorithms to the target and other data stored on the databases 142a-142c to generate one or more predictive models. The predictive model(s) can then be output to be used in other modules on the remote server 140, and/or can be output to the third party 180 (FIG. 1) to prompt further studies. Additional details on examples of the application of the AI/ML algorithms to the target and other data are discussed below with respect to FIG. 17.

In yet another example, the remote server 140 can include an additional module that uses a predictive model to evaluate a current developmental status of the supervised person 130. In some implementations, the remote server 140 develops a novel predictive model using the target data in another module (e.g., as discussed above) and applies the novel predictive model to the target data for a specific supervised person in the present additional module. For example, as discussed above, the remote server 140 can input the target data (and any other data) into an AI/ML to generate a predictive model, then apply the predictive model to the target data for the supervised person 130. Additionally, or alternatively, the remote server 140 can apply a developmental model and/or predictive model from one or more third parties 180 (FIG. 1) and/or any other suitable institution. Purely by way of example, the World Health Organization (“WHO”) has published an attachment classification framework that includes: secure; insecure-avoidant; insecure-ambivalent, anxious or resistant; and disorganized-disoriented. The remote server 140 can use the target data (e.g., assessments in the developmental data alongside linked bioindicator data) to identify a current classification the supervised person 130 under the WHO attachment classification framework. In another example, the remote server 140 can use the target data to determine which CDC-identified physical, emotional, and/or cognitive developmental milestones the supervised person 130 has achieved and compare their achievements to expectations for the supervised person 130 (e.g., based on their age).

In some implementations, the remote server 140 allows a responsible person (e.g., the controlling person 110) to indicate a preference for the source of the predictive model. Purely by way of example, the remote server 140 can allow a parent to select between predictive models from multiple third parties 180 (FIG. 1) and/or by the remote server 140, then use the selected predictive model in assessing the impact of changes and/or generating recommendations.

Once the current developmental status is identified, the remote server 140 can output the current developmental status of the supervised person 130 to any of the controlling person(s) 110, the supervising person(s) 120, and/or the third parties 180 (FIG. 1). The current developmental status can then be used to help make decisions about the daily activities of the supervised person 130, help make decisions about who is trusted with the responsibility over the supervised person 130, help identify interventions for the supervised person 130 to impact the developmental status of the supervised person 130, help prompt specific studies at the third parties 180 (FIG. 1), and the like.

Additionally, or alternatively, once the remote server 140 identifies a current developmental status of the supervised person 130, the remote server 140 can track the development of the supervised person 130 over time. Tracking the development of the supervised person 130 over time can include recording each developmental status output by another module, comparing developmental statuses to identify changes and/or identify trends in the developmental status, comparing one or more developmental statuses to an expected status for the supervised person 130 (e.g., checking whether the supervised person 130 has achieved expected developmental milestones for their age), and/or predicting future changes based on identified trends. In some implementations, the remote server 140 uses the trends and/or changes to help make recommendations to one or more of the responsible persons based on the tracked development, as discussed in more detail below.

In yet another example, the remote server 140 can include an additional module that uses a predictive model to assess the impact of indicated changes (e.g., changes to daily routines and/or activities) on the developmental status of the supervised person 130. For example, the responsible persons can indicate one or more changes that they are considering (e.g., changes in nutritional intake, changes in daily exercise, changes in nap cycles, changes in supervision, changes in learning time, and the like) and the remote server 140 can predict how the changes will impact the developmental status of the supervised person 130. Additionally, or alternatively, the remote server 140 can use a predictive model to generate recommendations for changes to intentionally impact the developmental status of the supervised person 130. Purely by way of example, when the remote server 140 determines that the supervised person 130 is behind developmental goals (e.g., has not achieved developmental milestones for their age), the remote server 140 can recommend interventions to the responsible persons. In a specific, non-limiting example, the remote server 140 can identify that a child is behind on cognitive development and recommend one or more cognitive exercises to the child’s parents and/or childcare providers to help improve the child’s development. In another specific, non-limiting example, the remote server 140 can identify negative trends in the developmental status of an elderly person and recommend various interventions (e.g., additional exercise, additional social time, cognitive exercises, medical treatments and the like) to address the negative trends.

In some implementations, the predictive model the remote server 140 uses comes from the output of another module (e.g., as discussed above). In other implementations, the predictive model the remote server 140 uses in the fourth module 248 comes from the third parties 180 (FIG. 1). In some implementations, as discussed above, the remote server 140 allows a responsible person (e.g., the controlling person 110) to indicate a preference for the source of the predictive model. Purely by way of example, the remote server 140 can allow a parent to select between predictive models from multiple third parties 180 (FIG. 1) and/or by the remote server 140, then use the selected predictive model in assessing the impact of changes and/or generating recommendations.

Additional details on examples of suitable communication between components of the system 100, functions of the system 100, and operation of the components of the system 100 are disclosed in U.S. Provisional Pat. Application No. 63/260,440 by Monica Plath, filed Aug. 19, 2021, and U.S. Provisional Pat. Application No. 63/247,692 by Monica Plath, filed Sep. 23, 2021, the disclosures of each which is incorporated herein in their entirety by reference.

### Example Subsystems According to Implementations of the Present Technology

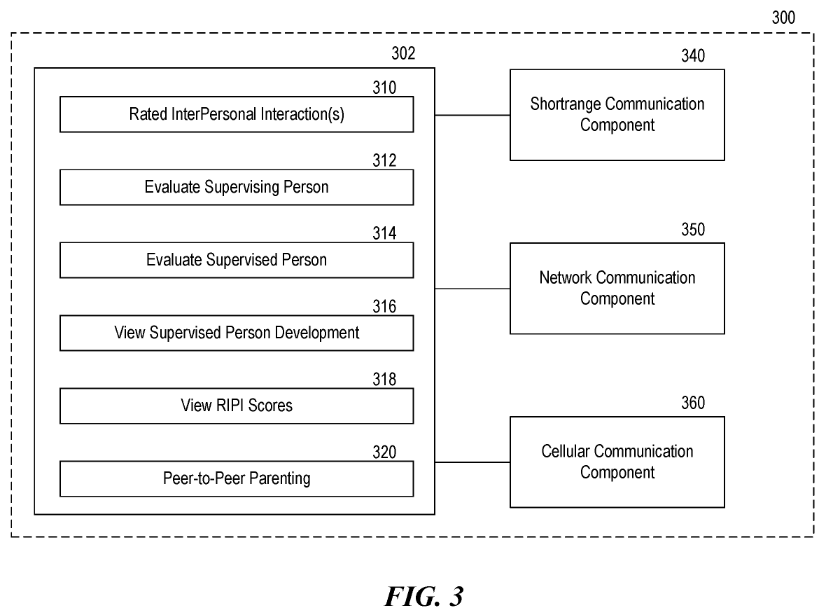

FIG. 3 is a schematic diagram of a subsystem 300 for a controlling person in the system for scoring supervising persons and tracking the development of supervised persons in accordance with some implementations of the present technology. The subsystem 300 can be deployed in the electronic device 112 discussed above with respect to the system 100 of FIG. 1. A processor and/or a storage component are not illustrated in FIG. 3 to avoid obscuring the illustrated components of the subsystem 300. However, one of skill in the art will understand that the subsystem 300 can include one or more processors and any suitable number of storage components to facilitate operation of the subsystem 300 as described herein.

As illustrated in FIG. 3, the subsystem 300 includes an operating platform 302 (“platform 302”) with one or more modules (six shown, referred to individually as first-sixth modules 310-320), a shortrange communication component 340, an internet communication component 350, and a cellular communication component 360. The shortrange communication component 340 can communicate over a short-range wireless standard (e.g., a Bluetooth®, Zigbee®, Z-Wave®, Wi-Fi HaLow®, or any other suitable short-range standard) to enable the subsystem 300 to communicate directly with other subsystems and devices that are within a local communication range. The internet communication component 350 enables the subsystem 300 to communicate with a network (e.g., the network 290 discussed above with respect to FIG. 2) over a wireless (or wired) internet connection (e.g., a WiFi connection or ethernet connection), allowing the subsystem 300 to connect with other subsystems and devices also connected to the network. Similarly, the cellular communication component 360 enables the subsystem 300 to communicate with the network through a cellular internet connection (e.g., based on a 3G, 4G, LTE, 5G, or other standard). The platform 302 is operably coupled to each of the shortrange communication component 340, the internet communication component 350, and the cellular communication component 360. Accordingly, any of the modules in the platform 302 can communicate with other subsystems and devices locally and/or over the network. Various examples of the modules are discussed in more detail below.

In the first module 310, a controlling person can provide evaluation data on an interaction with a supervised person and/or any other data related to the development of the supervised person. As discussed above, the evaluation data can include objective and/or subjective assessments of the interaction. In some implementations, the first module 310 includes prompts for the evaluation data. For example, the first module 310 can include prompts that inquire about information related to a standardized developmental model (e.g., the WHO classifications, CDC defined milestones, classifications and/or milestones identifies by a research group or other institution, and/or any other suitable standardized developmental model). The prompts can include inquiries about objective information (e.g., asking whether the child did or did not perform a certain behavior) and/or inquiries for subjective evaluations (e.g., ratings on a scale). The controlling person can access the first module 310 without being prompted after they have a relevant interaction with the supervised person. Additionally, or alternatively, the first module 310 can prompt the controlling person for an evaluation after a detected interaction. For example, the first module 310 can detect the presence of the supervised person for a predetermined period of time in coordination with the shortrange communication component 340, then prompt the controlling person for an evaluation.

In the second module 312, the controlling person can provide an evaluation of a supervising person. The evaluation of the supervising person can include objective and/or subjective assessments of the supervising person and/or their interactions with the supervised person. For example, the evaluation can indicate whether the supervising person performed various behaviors or actions in greeting the supervised person, an assessment of the rapport between the supervising person and the supervised person, an assessment of the controlling person’s satisfaction with the supervising person, and/or any other suitable evaluation. In some implementations, the evaluation of the supervising person can include responses to a standardized set of prompts, allowing the remote server 140 (FIG. 2) to more easily process the evaluations.

In the third module 314, the controlling person can provide an evaluation of a supervised person. The evaluation of the supervised person can include objective and/or subjective assessments of the supervised person after an interaction with another responsible person (e.g., after an interaction with the supervising person) or any other suitable time. In some implementations, the controlling person accesses the first module 310 to evaluate the supervised person after an interaction with the supervised person and accesses the third module 314 to evaluate the supervised person at any other time. For example, the controlling person can access the third module 314 after the supervised person achieves a developmental milestone outside of an interaction with the controlling person.

In the fourth module 316, the controlling person can view the developmental status and/or record of development for the supervised person that is output from the third module 246 (FIG. 2) on the remote server 140. In various implementations, the fourth module 316 can display a history of the developmental status, the current developmental status, and/or a prediction for the developmental status based on current trends. In some implementations, the fourth module 316 relays recommendations to the controlling person related to the development of the supervised person. In some implementations, the fourth module 316 displays an indication of various significant factors on the current developmental status. Purely by way of example, the fourth module 316 can indicate that the supervised person’s cognitive development is falling behind because the supervised person has not achieved various milestones expected for their age. In another example, the fourth module 316 can include an indication that one or more responsible persons have an especially positive and/or negative impact on the current developmental status. The indication of relative impacts can allow, for example, parents to identify when they (or a particular childcare provider) are holding their child’s development back in some way. The view of these indications is expanded on in the fifth module 318.

In the fifth module 318, the controlling person can view RIPI scores associated with one or more responsible persons. As discussed above, the RIPI score provides an indication of the impact the responsible person has on the persons under their supervision. For example, the RIPI score can indicate that a childcare provider has a positive (or negative) impact on the development of children under their supervision. In some implementations, the RIPI score can include various components indicating the impact the responsible person has on the persons under their supervision in various areas. By displaying the RIPI scores associated with the responsible person(s), the fifth module 318 allows the controlling person to make well-informed decisions about who they trust with the care and responsibility over the supervised person. The fifth module 318 also allows the controlling person to be more attentive to the development of the supervised person in areas indicated to be weaknesses for any of the responsible persons.

In the sixth module 320, the controlling person can connect other controlling persons (referred to as a “peer-to-peer network” of controlling persons) to share developmental tips regarding supervised persons and/or share information about one or more supervising persons. For example, a parent, acting as a controlling person, can access the sixth module 320 to review tips and/or testimony from other parents on how to handle a developmental issue they are experiencing with their child. In another example, the parent can access the sixth module 320 for peer-to-peer help with various parenting situations. In a specific example, a first parent may be especially good at teaching toddlers to read while a second parent is especially good at teaching the toddlers basic math. In this example, the two parents can connect through the sixth module 320 to share and take advantage of their relevant skill sets. The controlling person can also use the sixth module 320 to share reviews of public spaces, events, care providers, medical providers, and the like. For example, the controlling person can also use the sixth module 320 to share articles and/or view shared articles related to supervised persons (e.g., articles on child development). The controlling person can also use the sixth module 320 for various social purposes, such as to establish friend/trusted connections with other controlling persons in a social network framework (e.g., allowing parents on the system 100 (FIG. 1) to connect with other parents on the system 100 for any of the purposes discussed above).

In various implementations, the platform 302 can include one or more additional (or alternative modules). Purely by way of example, the platform 302 can include an additional module to upload developmental data related to the supervised person that is accessibly stored in the remote server(s). The developmental data can include assessments of the physical, emotional, cognitive, and/or social development of the supervised person; reports on the overall health of the supervised person; reports on the supervised person’s daily activities and/or experiences; observed developmental milestones; and the like. In some implementations, the developmental data is uploaded with a required security and/or permission level included. For example, uploading and/or viewing an evaluation of the emotional and/or mental development of a supervised person can be restricted to responsible persons with a preset connection security level and/or a preset permissions level (e.g., restricting uploading and reviewing evaluations of a child’s mental development to a head childcare provider and their parents).

The developmental data can include objective evaluations of the supervised person (e.g., whether the child made eye contact when saying hello; whether the child remembers the name of the responsible person; whether the child is able to walk and/or run on their own; and the like) and/or various subjective evaluations of the supervised person (e.g., rating, on a scale, the child’s comfort being away from a parent or guardian, being in a new environment, meeting new people, and the like). In some implementations, the additional module includes prompts for the developmental data. For example, the additional module can include prompts that inquire about information related to a standardized developmental model (e.g., the WHO classifications, CDC defined milestones, classifications and/or milestones identifies by a research group or other institution, and/or any other suitable standardized developmental model). The prompts can include inquiries about objective information (e.g., asking whether the child did or did not perform a certain behavior) and/or inquiries for subjective evaluations (e.g., ratings on a scale). The controlling person can access the additional module without being prompted after they have a relevant interaction with the supervised person or observe a relevant behavior. Additionally, or alternatively, the first module 310 can prompt the controlling person for an evaluation after a detected interaction (e.g., using proximity data) and/or after a relevant time period (e.g., for a quarterly update).

In another example, the platform 302 can include an additional module to provide other data related to the developmental status of the supervised person. As discussed above, the other data can include such as known allergies, known medical conditions, medical history information, known behavioral patterns, recent developments or updates, known mental impairments, and/or various other data that impacts the development of the supervised person. Non-limiting examples of medical history information can include information on vaccinations, family medical history, diagnoses specific to the supervised person, past medical events such as surgeries, illnesses, and/or major medical events (e.g., seizures). Non limiting examples of recent developments or updates include recent diagnoses, broken bones and/or other physical trauma, recently experienced mental and/or emotional trauma such as the loss of a family member, cognitive and/or behavioral developments such as learning to use the restroom for toddlers and loss of memory in adults, and the like.

In yet another example, the platform 302 can include an additional module to view the developmental status of the supervised person through access to the remote server 140 (FIG. 2). In some implementations, the additional module allows the controlling person to view a history of the developmental status along with (or in alternative to) the current developmental status. Providing access to the current developmental status and the history of the developmental status can help the controlling person monitor and track the development of the supervised person and supplement their own evaluations of the supervised person. Further, providing access to the current developmental status and the history of the developmental status can allow the controlling person to take interventive steps before large departures from a desired developmental status.

In some implementations, the additional module displays an indication of various significant factors on the current developmental status. Purely by way of example, the additional module can indicate that the supervised person’s cognitive development is falling behind because the supervised person has not achieved various milestones expected for their age. In another example, the additional module can include an indication that one or more responsible persons have an especially positive and/or negative impact on the current developmental status. The indication of relative impacts can allow, for example, parents to identify when they (or a particular childcare provider) are holding their child’s development back in some way.

In yet another example, the platform 302 can include an additional module to allow the controlling person to provide any number (including zero) proposed changes and view a prediction for how the changes (or lack thereof) will impact the developmental status of the supervised person over time. In some implementations, the additional module provides a standardized format for indicating proposed changes based on various types of changes (e.g., to provide a standardized form to indicate dietary changes, changes in exercise, changes in education instruction, changes in sleep schedules, and the like), allowing the changes to be sent to and quickly processed by the remote server 140 (FIG. 2). In various implementations, the prediction for how the changes (or lack thereof) will impact the developmental status can be near term and/or far term. For example, the prediction can include an indication of the immediate impact of a change on the developmental status of the supervised person as well as how the change will impact the developmental status half a year later, a year later, five years later, ten years later and/or after any suitable period of time. Purely by way of example, a change may indicate an increase in cardio exercise for a toddler, and the prediction can indicate that the child is likely to become more irritable immediately after the change as they adjust to the increased physical exertion (e.g., reducing their social developmental status), but that the toddler will adjust over time and that the change will increase their physical and/or cognitive development a year or more after the change is made

In yet another example, the platform 302 can include an additional module to allow the controlling person to view one or more recommendations from the predictive models on the remote server 140 (FIG. 2) related to the developmental status of the supervised person. For example, the remote server 140 can determine that the supervised person is falling behind on their developmental status in one or more areas and generate recommendations for changes to accelerate their development. The controlling person can then view the recommendations through the additional module. Purely by way of a simple example, the remote server 140 can determine that a toddler is behind on cognitive development and recommend one or more daily exercises to accelerate their cognitive development. In some implementations, the controlling person can view a predicted impact and/or predicted timeline for the impact through the additional module. Returning to the simple example above, the parent of the toddler can view how much impact the daily exercises are likely to have (e.g., whether the changes will catch the toddler back up to expected development, eventually advance the toddler beyond an expected development, prevent the toddler from falling farther behind, and the like), and/or a timeline for the likely impacts (e.g., that the toddler will be caught up within a month, within half a year, within a year, and the like).

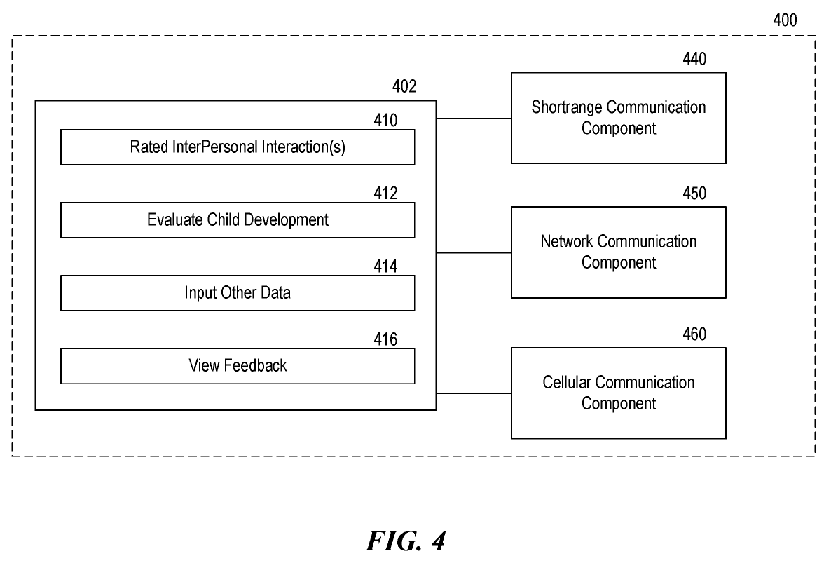

FIG. 4 is a schematic diagram of a subsystem 400 for a supervising person in the system scoring supervising persons and tracking the development of supervised persons in accordance with some implementations of the present technology. The subsystem 400 can be deployed in the electronic device 112 discussed above with respect to the system 100 of FIG. 1. Like the subsystem 300 discussed above with respect to FIG. 3, the subsystem 400 includes an operating platform 402 (“platform 402”) with one or more modules (four shown, referred to individually as first-fourth modules 410-416), a shortrange communication component 440, an internet communication component 450, and a cellular communication component 460. Further, the platform 402 is operably coupled to each of the shortrange communication component 440, the internet communication component 450, and the cellular communication component 460, allowing the modules in the platform 402 to communicate with other subsystems and devices locally and/or over the network. Various examples of the modules are discussed in more detail below.

In the first module 410, the supervising person can provide evaluation data on an interaction with a supervised person and/or any other data related to the development of the supervised person. As discussed above, the evaluation data can include objective and/or subjective evaluations of the interaction. In some implementations, the first module 410 includes prompts for the evaluation data. For example, the first module 410 can include prompts that inquire about information related to a standardized developmental model (e.g., the WHO classifications, CDC defined milestones, classifications and/or milestones identifies by a research group or other governmental body, and/or any other suitable standardized developmental model). The prompts can include inquiries about objective information (e.g., asking whether the child did or did not perform a certain behavior) and/or inquiries for subjective evaluations (e.g., ratings on a scale). The supervising person can access the first module 410 without being prompted after they have a relevant interaction with the supervised person. Additionally, or alternatively, the first module 410 can prompt the supervising person for an evaluation after a detected interaction. For example, the first module 410 can detect the presence of the supervised person for a predetermined period of time in coordination with the shortrange communication component 440, then prompt the supervising person for an evaluation.

In the second module 412, the supervising person can provide an evaluation of the supervised person outside of an interpersonal interaction. The evaluation of the supervised person can include objective and/or subjective assessments of the supervised person based on events outside of interpersonal interactions and/or outside a single interpersonal interaction. In a specific, non-limiting example, the evaluation can reflect on the supervised person’s developmental status during a quarter, semester, summer camp, daycare period, and/or any other suitable period. In another specific example, the supervising person can access the second module 412 after observing an interaction between the supervised person and another person (e.g., another supervised person) and/or after a significant event (e.g., after a field trip, after a stressful event, etc.). In some implementations, the controlling person accesses the first module 410 to evaluate the supervised person after an interaction with the supervised person and accesses the second module 412 to evaluate the supervised person at any other time. For example, the controlling person can access the second module 412 after the supervised person achieves a developmental milestone outside of an interaction with the supervising person.

In the third module 414, the supervising person can input any other data related to the supervised person. For example, the other data can include pictures and /or videos of the supervised person, indications of medical treatment (e.g., a report of the dosages and types medications given to the supervised person after an incident, a report of the treatments given to the supervised person after an incident, and the like), reports on what foods the supervised person consumed while under the responsibility of the supervising person, reports on the sleep (e.g., via naps) the supervised person had while under the responsibility of the supervising person, and/or any other suitable information related to tracking the health and development of the supervised person.

In the fourth module 416, the supervising person can view feedback on their performance as a supervising person. The feedback can include their RIPI score, an indication of how their RIPI score was calculated, suggestions for improving their RIPI score, articles related to their RIPI score, comments and/or reviews from the controlling person(s), and the like. The fourth module 416 allows the supervising person to understand how they may be impacting the development of the supervised persons under their responsibility, understand and address their weaknesses, and/or better communicate with controlling person(s) about their strengths and weaknesses. For example, the fourth module 416 could allow a supervising person to understand how they may be negatively impacting emotional development of supervised persons under their responsibility, find training for improving their impact, and be ahead of questions from controlling persons about their impact on emotional development.

In various implementations, the platform 402 can include one or more additional (or alternative modules). For example, the platform 402 can include additional modules similar to any of those discussed above with reference to FIG. 3. In a specific, non-limiting example, a supervising person can provide developmental data on one or more supervised persons (e.g., based on interactions, observed milestones, periodic evaluations, and the like). In another specific, non-limiting example, the supervising person can provide other data related to the developmental status of the one or more supervised persons. In yet another specific, non-limiting example, the supervising person can view the developmental status of the one or more supervised persons through access to the remote server 140 (FIG. 2), along with trends in the developmental status of the one or more supervised persons. In yet another specific, non-limiting example, the supervising person can provide any number (including zero) proposed changes and view a prediction for how the changes (or lack thereof) will impact the developmental status of the one or more supervised persons over time. And in yet another specific, non-limiting example, the supervising person can view one or more recommendations from the predictive models on the remote server 140 related to the developmental status of the one or more supervised persons.

Similar to the benefits discussed above, the access to information about the developmental status for the one or more supervised persons provided by the platform 402 can allow the supervising person to better monitor the supervised persons they are responsible for, make changes to their care and/or supervision of the supervised persons, and/or make better recommendations to controlling persons for changes to the daily lives of the supervised persons. In some implementations, the predictions from the remote server 140 (FIG. 2) and viewed through the platform 402 are made on a broadly applicable level. For example, the supervising person can indicate a change in the food they provide to supervised persons under their responsibility, and view a broad-level prediction for how the change is likely to impact the supervised persons (e.g., a prediction that the supervised persons, on average, will be more healthy after the change). In another example, the predictive model generated by the remote server 140 (FIG. 2) can identify one or more activities are correlated with positive developmental statuses, and recommend that the supervising persons make changes to include the identified activities into the daily life of the supervised persons.

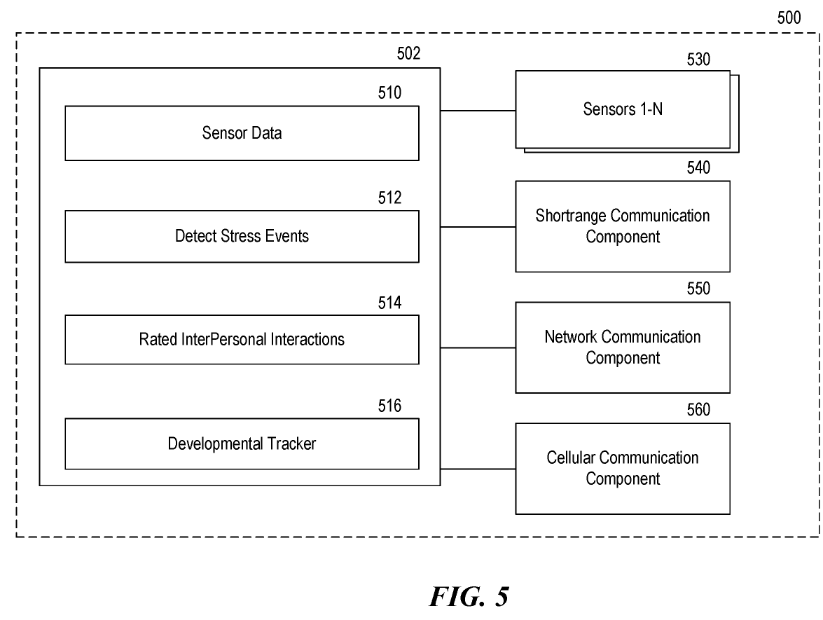

FIG. 5 is a schematic diagram of a subsystem 500 for a wearable device for use by a supervised person in the system for scoring supervising persons and tracking the development of supervised persons in accordance with some implementations of the present technology. The subsystem 500 can be deployed in the wearable device 132 discussed above with respect to the system 100 of FIG. 1.

Like the subsystems 300, 400 discussed above with respect to FIGS. 3 and 4, the subsystem 500 can include an operating platform 502 (“platform 502”) with one or more modules (four shown, referred to individually as first-fourth modules 510-516), a shortrange communication component 540, an internet communication component 550, and a cellular communication component 560. Further, the subsystem 500 can include on or more sensors 530 (1-N indicated) that collect bioindicators while worn by the supervised person. Purely by way of example, the sensors 530 can include a PPG sensor, an accelerometer, a skin temperature sensor, a skin conductivity sensor, additional hydration sensors, heart-rate variability sensors, resting heart rate sensors, sweat chemical composition sensors, nervous system electrical sensors, air quality sensors, UV exposure sensors, sensors to detect environmental chemicals, blood oxygen and/or pulse oxygen sensors, voice recognition, electrocardiogram (ECG) sensor, pressure sensors, gyroscopes, magnetometers, and/or any combination therein. The PPG sensor allows the subsystem 500 to measure and record the supervised person’s heart rate; the accelerometer allows the subsystem 500 to measure and record the supervised person’s movement; the skin temperature sensor allows the subsystem 500 to measure and record the supervised person’s temperature over time; and the skin conductivity sensor allows the subsystem 500 to measure and record the supervised person’s level of psychological or physiological arousal, which is effected by the supervised person’s cognitive activity and/or emotions.

As further illustrated in FIG. 5, the platform 502 is operably coupled to each of the one or more sensors 530, the shortrange communication component 540, the internet communication component 550, and the cellular communication component 560, allowing the modules in the platform 502 to communicate with other subsystems and devices locally and/or over a network. For example, the platform 502 can control the shortrange communication component 540 to detect other subsystems (e.g.., by sending and/or receiving presence detection signals) within a range of the shortrange communication component 540. Additionally, or alternatively, the platform 502 can control the shortrange communication component 540, the internet communication component 550, and/or the cellular communication component 560 to communicate information from the one or more sensors 530 to the remote server.

The first module 510 allows the platform 502 to receive, organize, store, and/or communicate the data from the sensors. The data can include numerous different bioindicators, such as any of the bioindicators from the sensors discussed above. In some implementations, the first module 510 also at least partially processes and/or links corresponding portions of the bioindicator data. For example, the first module 510 can receive data from a skin conductivity sensor, process the data to determine related cognitive activity and/or emotions, then communicate and/or store the determined cognitive activity and/or emotions. In another example, the first module 510 can link bioindicator data from each of the sensors measured during a relevant period, for example allowing the related bioindicator data to be later reviewed and/or processed together.

The second module 512 allows the platform 502 to detect stress events locally (e.g., without sending the sensor data to the remote server 140 (FIG. 2)). For example, the platform 502 can detect when the data from a skin conductivity sensor is indicative of a stress event, especially when data from any of the other sensors 530 corroborates the stress event (e.g., through data indicating an elevated blood pressure, elevated pulse, and the like). In some implementations, once the platform 502 detects a stress event, the platform 502 queries the sensors 530 for data more frequently to more completely track bioindicators around the stress event. In some implementations, once the platform 502 detects a stress event, the platform 502 sends a notification to the supervising person 120, the controlling person 110, and/or the remote server 140. The notification can prompt the responsible person to pay extra attention to the supervised person, prompt the responsible person to provide an evaluation of the supervised person during and/or after the stress event, and/or prompt the responsible person to provide an explanation for the stress event. Similarly, the notification can prompt the remote server 140 to inquire about details from the responsible person; evaluate the developmental status of the supervised person based on evaluation data and/or bioindicator data received around the stress event; and/or update a RIPI score for the responsible person based on the occurrence of the stress event, the explanation for the stress event provided by the responsible person, the evaluation data provided by the responsible person, and/or the bioindicator data received around the stress event.

The third module 514 allows the platform 502 to provide data related to an interpersonal interaction (e.g., provide bioindicator data during the interaction). The platform 502 can access the third module 514 when prompted by the remote server 140 (FIG. 2) and/or either of the responsible persons. Additionally, or alternatively, the platform 502 can access the third module 514 after detecting the presence of a responsible person (e.g., through the shortrange communication component 540) for more than predetermined period of time and/or after detecting a change in the presence of a responsible person.

The fourth module 516 allows the platform 502 to provide specific bioindicator data related to the development of the supervised person. The platform 502 can access the fourth module 516 when prompted by the remote server 140 (FIG. 2) and/or either of the responsible persons. For example, the remote server 140 can determine that additional data (e.g., a record of activity) would be necessary (or helpful) in determining a current developmental status for the supervised person and prompt the subsystem 500 to provide the data. Additionally, or alternatively, the platform 502 can access the fourth module 516 to provide periodic, or continuous, updates on the bioindicators that can later be used to assess the development of the supervised person. Purely by way of example, the updates can provide a record of the exercise the supervised person gets daily, weekly, monthly, and/or during any other suitable period, as well as their bioindicators while exercising (e.g., heartrate), which in turn can be used to assess various aspects of their development. In some implementations, If the supervised person has not reached their daily goal before a predetermined time (e.g., by 3:00 PM or any other suitable time), the platform 502 can communicate an alert to the responsible person to prompt additional exercise. In another example, the platform 502 can access the fourth module 516 to track whether the supervised has had a predetermined amount of mental stimulation for a day (e.g., based on the skin conductivity data). If the supervised person has not reached their daily goal before a predetermined time, the platform 502 can communicate an alert to the responsible person to prompt additional mental stimulation.

In various implementations, the platform 502 can include one or more additional, or alternative modules. Purely by way of example, the platform can include an additional module that maintains a baseline for the bioindicator data expected in various situations. In a specific example, the additional module can maintain a record of a baseline heartrate (e.g., resting heart rate, typical heart rate during interactions, during exercise, and the like), skin temperature, skin conductivity, stress levels, and the like. In some implementations, a departure from the baseline can trigger one or more of the modules discussed above, and/or can trigger the platform to more closely monitor the supervised person. For example, when an elevated heart rate is identified, the platform can control the sensors on the wearable device 132 (FIG. 1) to more closely monitor bioindicator data for the supervised person. The additional data can help the platform 502 identify a cause of the departure, alert one or more responsible persons of the departure, and the like.

### Examples of Systems and Methods Related to Interpersonal Interactions According to Implementations of the Present Technology

FIG. 6 is a schematic view of a series of rated interpersonal interactions (RIPIs) 601 in accordance with some implementations of the present technology. In the illustrated implementation, the RIPIs occur between one or more supervising persons 120 (two shown) and one or more supervised persons 130 (three shown). The RIPIs 601 include interactions such as a greeting between the supervising persons 120 (e.g., childcare providers) and the supervised persons 130 (e.g., children) when the supervised persons 130 are dropped off, interactions between the supervised persons 130 when left alone in a room, how the supervised persons 130 interact with the supervising persons 120 when receiving a snack or treat, how the supervised persons 130 react to separation from the controlling persons (e.g., after a drop-off when their parent or guardian leaves), how the supervised persons 130 react in response to the controlling persons returning, and any other suitable interaction. The interactions are rated by the evaluations the childcare providers submit to the remote server 140 (FIG. 2), the bioindicator data from any wearable devices on the supervised persons 130, and/or an analysis by the remote server 140 of the evaluation data and the bioindicator data.

The large number of RIPIs 601 can be helpful in setting a CR baseline for how the supervised persons 130 behave and interact with other persons, setting a CG baseline reflecting a personality for the supervising persons 120, and/or setting an E baseline for how the supervising persons 120 typically evaluate the supervised persons 130 under their supervision. Once the baselines have been set, the large number of RIPIs can be helpful in accurately assessing the developmental status of each of the supervised persons 130 and/or generating a RIPI score for the supervising persons 120.

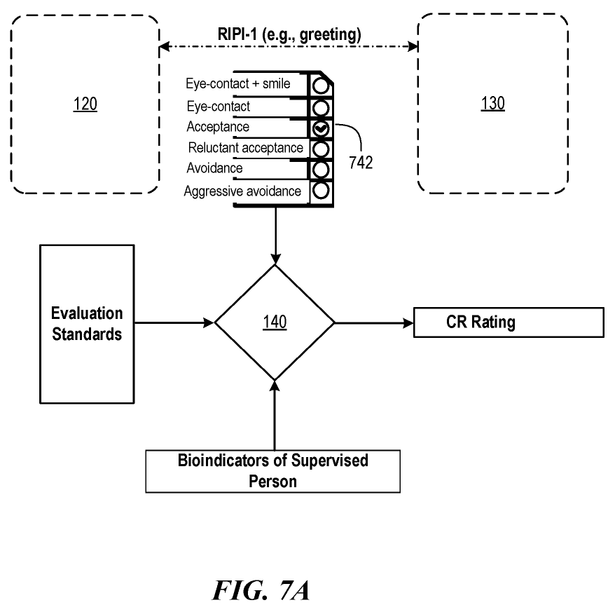

FIG. 7A is a schematic view of a process for rating a supervised person after a single interpersonal interaction in accordance with some implementations of the present technology. As illustrated in FIG. 7A, the relevant interaction includes a greeting between a supervising person 120 (e.g., illustrated as a childcare provider) and a supervised person 130 (e.g., illustrated as a toddler). As illustrated, the supervising person 120 submits an evaluation 742 of the supervised person 130 based on their behavior during the interaction that includes various objective assessments of the supervised person’s behavior during the interaction (e.g., made eye contact and smiled, made eye contact, and/or any other suitable indication), and/or subjective assessments of the supervised person’s behavior during the interaction (e.g., indicating one of: accepted the care of supervising person 120, was reluctant to accept the care of supervising person 120, avoided the care of the supervising person 120, aggressively avoided the care of the supervising person 120).

The evaluation 742 is sent to a the remote server 140 to process the RIPI data (e.g., via the first module 242 (FIG. 2) on the server 140), along with various baselines for the persons in the interaction and bioindicator data from a wearable sensor on the supervised person 130. In the illustrated implementation, the system then outputs a CR rating of the supervised person 130 during the interaction. The CR rating can then be used to assess the developmental status of the supervised person 130 and/or update the CR baseline for the supervised person 130.

FIG. 7B is a schematic view of the process of FIG. 7B over a series of interpersonal interactions in accordance with some implementations of the present technology. Similar to the single interaction discussed above with respect to FIG. 7A, the relevant interaction includes a greeting between the supervising person 120 and the supervised person 130, after which the supervising person 120 submits an evaluation 742 of the supervised person 130. The evaluation 742 includes various objective and/or subjective assessments. The evaluation 742, any existing baselines for the persons in the interaction (e.g., the supervising person 120 may have an established E baseline and/or CG baseline while the supervised person 130 is new to the system), and/or bioindicator data from a wearable sensor on the supervised person 130 are then sent to the remote server 140 to process the RIPI data. Over a series of interactions, the system can generate a CR baseline for the supervised person 130 that can then be used in processing later RIPIs. In a specific example, the CR baseline can indicate an expected behavior for a child during greetings, such as an expectation that the child will not make eye contact, will smile, and will be slightly reluctant to leave their parents. Any departure from the expected behavior, such as no reluctance to leave their parent, can help indicate developmental process for the child.

FIG. 8 is a flow diagram of a process 800 for scoring a supervised person after a single interpersonal interaction in accordance with some implementations of the present technology. The process 800 can be at least partially executed by a module on the cloud sever 140 described above with respect to FIG. 2 to process and evaluate interactions between persons in the system.

In the illustrated implementation, the process 800 begins at block 802 by detecting an interpersonal interaction (and/or a set of interactions) between a responsible person and a supervised person. In various implementations, the interaction can be detected by receiving a presence detection indication from the responsible person and/or the supervised person (e.g., when the electronic devices 112 and wearable device 132 (FIG. 1) sense the presence of each other using their shortrange communication components), evaluating location data from the responsible person and/or the supervised person (e.g., processing the GPS data from the responsible person and the supervised person to identify an overlap within a predetermined distance), receiving an indication from the responsible person that an interaction occurred, a reoccurring calendar trigger (e.g., after a scheduled drop-off (e.g., morning drop off), a schedule takeover (e.g. when one care provider goes on break), and the like, and/or any other suitable detection means.

In some implementations, after detecting the interpersonal interaction, the process 800 sends a prompt to the wearable device and/or the electronic device associated with the responsible person. The prompt can include instructions for the wearable device to communicate relevant bioindicator data (e.g., bioindicator data during the interpersonal interaction, baseline data for the supervised person, and the like). Additionally, or alternatively, the prompt can include instructions for the responsible person to evaluate the supervised person. For example, the instructions can query the responsible person for objective data (e.g., whether various behaviors were observed, whether various milestones have been observed, and the like). In another example, the instructions can query the responsible person for subject evaluations of the supervised person (e.g., to evaluate stress levels, engagement, mood, energy levels, and the like). In some implementations, the instructions vary based on a type of the detected interpersonal interaction. For example, if the detected interpersonal interaction is the first for a given day, the instructions can query the responsible person for evaluations associated with greeting the supervised person and/or the departure of another responsible person (e.g., the departure of a parent when they drop off a toddler at daycare). In another example, the instructions can vary based on a length of the detected interpersonal interaction.

At block 804, the process 800 includes receiving bioindicator data from the supervised person. The bioindicator data can be received through the responsible person (e.g., when the wearable device communicates the bioindicator data using the shortrange communication component) and/or through a network connection. In some implementations, the bioindicator data includes periodic updates on the bioindicators of the supervised person throughout the detected interpersonal interaction. In some implementations, the bioindicator data includes a continuous record of the bioindicators of the supervised person throughout the detected interpersonal interaction.

At block 806, the process 800 includes receiving an evaluation of the supervised person. As discussed above, the evaluation can include various objective and/or subjective assessments of the supervised person’s behavior during the detected interaction. Purely by way of example, the evaluation can include assessments of whether the supervised person made eye contact, smiled, cried, spoke, expressed object permanence, and/or exhibited various other behaviors. In another example, the evaluation can include assessments of the level of acceptance (or avoidance) the supervised person expressed for the supervising person (e.g., rating the supervised person’s comfort level away from the controlling person and/or with the supervising person on a scale). In various implementations, the assessment can include various objective data (e.g., whether certain behaviors or milestones were observed) and/or various subjective data (e.g., a rating of the supervised person’s mood, stress levels, energy level, and the like). In some implementations, the evaluation includes data from multiple responsible persons (e.g., both a controlling person and a supervising person, multiple supervising persons, and the like). In some such implementations, the evaluation is retrieved altogether. In other implementations, the process 800 retrieves a first portion associated with a first responsible person during a first pass through blocks 804-816 and retrieves a second portion associated with a second responsible person during a second pass through blocks 804-816.

At block 808, the process 800 includes checking the bioindicators of the supervised person for contradictions (or corroborations) with the data in the evaluation. Purely by way of example, the evaluation can include an assessment of the supervised person’s mood during the interaction (e.g., whether a child was calm or stressed during separation from their parent), which can be at least partially contradicted and/or corroborated by the bioindicators of the supervised person (e.g., by the supervised person’s heart rate, skin conductivity, skin temperature, neurological signals, and the like). In another example, the evaluation can include an assessment of the supervised person’s behavior that includes an indication of movement from the supervised person (e.g., that a child ran back to their parent) that can be at least partially contradicted (or corroborated) by the bioindicators of the supervised person (e.g., data denying (or confirming) movement during the interaction, such as movement data, heart rate data, position and orientation data, and the like). In yet another example, the evaluation can include an indication of an objective physical development (e.g., that a toddler walked or ran for the first time) that can be at least partially contradicted (or corroborated) by the bioindicators (e.g., movement data, position and orientation data, and the like indicating no movement during the interaction).

In some implementations, checking the bioindicator data for contradictions (or corroborations) can include comparing the bioindicator data during the relevant interaction to baselines for the supervised person. The baselines can be stored on the wearable device (e.g., communicated when prompted, used to filter the bioindicator data communicated, or communicated during the interaction) and/or by the cloud server (e.g., received from the wearable device or generated based on a history of bioindicator data). In a specific, non-limiting example, checking for the bioindicator data for contradictions (or corroborations) can include comparing a measured heart to a baseline (e.g., resting) heart rate. An elevated heart rate can help indicate that the supervised person was not calm (e.g., stressed, mad, and the like) during the interaction. In some implementations, checking the bioindicator data for contradictions (or corroborations) can include tacking the bioindicator data throughout the interaction to measure fluctuations. In a specific, non-limiting example, an escalating heart rate (e.g., compared to resting) during the interaction may indicate increasing stress in the supervised person (e.g., increasing stress in a toddler as a parent leaves).

At decision block 810, if a contradiction was found, the process 800 can continue to block 812 to address the contradiction, else the process 800 can continue to block 814.

At block 812, the process 800 includes prompting the supervising person for a reevaluation of the interaction and/or an explanation for the contradiction. In some implementations, the prompt includes an indication of the detected contradiction to help direct the supervising person’s attention to what assessments to revisit. In some implementations, the process 800 then returns to blocks 806-810 to receive the reevaluation of the supervised person and check for remaining contradictions between the reevaluation (or explanation) and the bioindicators. In a specific, non-limiting example, an elevated heart rate (e.g., compared to resting) at the start of the interaction may indicate stress in the supervised person that is evaluated as having a calm mood. However, if their heart rate drops through the interaction, the decline can help corroborate an explanation that the supervised person exercised before the interaction, allowing the process 800 to dismiss the contradiction between the evaluation and the bioindicator data. In some implementations, the process 800 receives the reevaluation at block 812 and proceeds directly to block 814.

Additionally, or alternatively, the process 800 can include parsing the evaluations from multiple responsible persons for contradictions (or corroborations) in blocks 808-812. For example, at block 808, the process 800 can include checking for contradictions (or corroborations) in the evaluations from multiple supervising persons; at decision block 810, if a contradiction was found between the evaluations, the process 800 continues to block 812; and at block 812, the process 800 can include prompting one or more of the supervising persons for an explanation of the contradiction and/or a reevaluation of the of the supervised person. In some implementations, if a contradiction is found, the process 800 includes checking the multiple evaluations for consistency with the bioindicator data. The process 800 can then discard the evaluation that is not consistent with the bioindicator data prompt the supervising persons associated with the inconsistent evaluation for a reevaluation of the of the supervised person; and/or preference the data in the consistent evaluation. In some implementations, if a contradiction is found, the process 800 includes checking whether one of the evaluations is associated with a high ranking and/or more qualified supervising person. Purely by way of example, a supervisor at a care providing facility can be assigned a higher ranking than their subordinates. The process 800 can then discard the evaluation from the lower ranking supervising person; prompt the lower ranking supervising person a reevaluation of the of the supervised person; and/or preference the data associated with the higher-ranking supervising person.

In some implementations, the process 800 can record the number of contradictions between an evaluation and the bioindicators, record the type of contradictions (e.g., that all the contradictions were related to the stress levels of the supervised person), record the number of iterations the process 800 must follow between blocks 806 and 812 before the contradictions are resolved, and/or various other metrics. The metrics can then be used in other processes when evaluating the supervising person. For example, a record showing numerous iterations between blocks 806 and 812 can negatively impact an assessment of the supervising person.

At block 814, the process 800 includes generating a CR rating for the supervised person based on the evaluation from the supervising person and/or the bioindicator data. Purely by way of example, the CR rating can be calculated using a weighted polynomial equation. In a specific, non-limiting example, the weighted polynomial equation can have a term for each of the CDC milestones for an age of the supervised person, where the weight assigned to each of the milestones can be partially dependent on the evaluation data (e.g., the evaluation data can include a “not applicable” response for one or more milestones that adjusts a weight assigned to a milestone to zero; the weights for each milestone can be dependent on a total number of milestones being considered, and the like). In the specific example, the value associated with objective data (e.g., whether the supervised person made eye contact) can be either 0 or 1 while the value associated with subjective data (e.g., an evaluation of the supervised person’s mood, stress, energy levels, and the like) can range between 0 and 1. The CR rating can reflect whether the supervised person exhibited certain behaviors expected of them (e.g., based on guidelines from WHO, the CDC, an academic research framework, and/or any other suitable framework). Purely by way of example, the CDC cognitive milestones indicate that a four-year-old should be able to correctly use gender pronouns, tells stories, say their first and last name, understand the idea of counting, play basic board games and card games, name some colors and numbers, have a basic understanding of time, and various other milestones. The evaluation from the supervising person can include assessments directed to these milestones that are then reflected in the CR rating. In another example, the WHO has published attachment classifications that can be used to help evaluate the social and/or emotional development of a supervised person. The classifications include secure; insecure-avoidant; insecure-ambivalent, anxious or resistant; and disorganized-disoriented. The evaluation from the supervising person can include assessments directed to these classifications and/or related personality traits (e.g., that the supervised person is shy but otherwise secure) that are then reflected in the CR rating.

In some implementations, the process 800 associates the CR rating with a confidence level. The confidence level reflects how likely the CR rating is to be accurate based on the RIPI data used to generate the CR rating and/or the persons involved. In various implementations, the confidence level can be at least partially dependent on the number of contradictions between the evaluation data and the bioindicator data, the number of objective assessments, the number of subjective assessments, the amount of data in the RIPI data, and/or a trust score for the supervising person. For example, in general, the less RIPI data that is available to generate the CR rating (e.g., from fewer assessments in the evaluation data), the lower the confidence level the process 800 will assign to the CR rating. One exception, for example, is when the evaluation data includes objective assessments that are determinative, or at least partially determinative, of the CR rating. In such instances, the process 800 can have assign a high confidence level to the CR rating despite limited data. In another exception, the confidence level can remain low despite a large number of assessments when the assessments are primarily subjective assessments and/or when a confidence score for the supervising person is low. The trust score for the supervising person can be low, for example, when the supervising person does not have an established baseline themselves, or when the assessments from the supervising person are known to often differ from the assessments of other responsible persons.

In some implementations, the confidence level can be at least partially dependent on a qualification status for the responsible person providing the evaluation. Purely by way of example, the process 800 can associate a CR rating resulting from an evaluation from a medical care professional with a high confidence level, or can associate a CR rating resulting from an evaluation from a relatively new care provider can be given a low confidence level. In another example, the process 800 can associate the CR rating from an evaluation submitted by a supervisor at a care providing facility can be given a higher confidence level than an evaluation submitted by a subordinate at the care providing facility.

In some implementations, the confidence level can be at least partially dependent on the number and/or source(s) of contradictions and/or agreements between multiple responsible persons submitting an evaluation. For example, when both a supervising person and a controlling person submit evaluations that corroborate each other, the process 800 can associate the CR rating with a high confidence level. In another example, where two or more supervising persons submit evaluations that corroborate each other, the process 800 can associate the resulting CR rating with a high confidence level. Conversely, where two responsible persons submit evaluations that contradict each other, the process 800 can associate the resulting CR rating with a low confidence level unless the contradictions are satisfactorily addressed at block 812.

At block 816, the process 800 includes outputting the CR rating. The output can be stored to one of the databases 142a-142c (FIG. 2) in the remote server 140, used in one or more additional modules on the remote server 140 and/or additional processes in the system, and/or shared with one or more responsible persons (e.g., shared with the controlling person associated with the supervised person, shared with one or more supervising persons, shared with an institutional care provider for use in assigning supervising persons, and the like). In some implementations, the output at block 816 can trigger the process 900 described below with respect to FIG. 9.

FIG. 9 is a flow diagram of a process 900 for monitoring and aiding the development of a supervised person interaction in accordance with some implementations of the present technology. The process 900 can be at least partially executed by a module on the cloud sever 140 described above with respect to FIG. 2 to process and evaluate interactions between persons in the system.

In the illustrated implementation, the process 900 begins at block 902 by receiving the CR rating. In some implementations, the CG rating is received directly from the process 800 of FIG. 8. In some implementations, the CR rating is received from a database (e.g., one of the databases 142a-142c (FIG. 2) in the remote server 140) when a responsible person prompts the system to execute the process 900.

At block 904, the process 900 includes checking the total number and quality of CR ratings. The quality of the CR rating can be at least partially dependent on a confidence level associated with the CR rating. The total number of CR ratings is important to ensure a sufficient number of CR ratings have been received to generate an accurate score and/or to ensure that a single overly good/bad CR rating does not skew the result of the process 900. Similarly, having a sufficient quality of CR ratings is important to generate an accurate score. For example, a large number of CR ratings may not provide an accurate representation of the supervised person when the confidence level for each of the CR ratings is low. Conversely, a smaller number of CR ratings may be necessary when the confidence level in each CR rating is high.

At decision block 906, if there are sufficient CG ratings (e.g., number and quality) already received for the supervised person, the process 900 continues to block 910; else the process 900 continues to block 908.

At blocks 908 and 910, the process 900 includes updating a CR baseline for the supervising person. Purely by way of example, the CR baseline can be calculated using a weighted polynomial equation. In a specific, non-limiting example, the weighted polynomial equation can have a term for each of the CR ratings, where the weight assigned to each of the CR ratings can be partially dependent on a confidence score associated with each rating and/or n E rating associated with the evaluators (discussed in more detail below). The CR baseline can be used in processing the RIPI data to adjust expectations for the interactions and/or the evaluations from the interactions. Purely by way of example, the CR baseline can indicate that a supervised person is generally shy, such that deviations from the CR baseline (e.g., indicating an outgoing interaction with the supervised person) can indicate significant change for the supervised person, a significant impact from the supervising person, and/or an error in the evaluation. In another example, the CR baselines for a number of supervised persons can be factored into the generation of the RIPI score for the supervising person in block 1210 of FIG. 12, discussed below. Additionally, or alternatively, the CR baseline can also help the process 900 efficiently evaluate the developmental status of the supervised person by keeping a running and/or weighted record of various aspects of their development before they have had sufficient CR ratings to fully and/or accurately evaluate their developmental status.

The update to the CR baseline reflects the additional data point received through the CR rating for the interaction. For example, a supervised person may be uncharacteristically shy and/or avoidant during a first interaction, then more outgoing and/or secure in subsequent interactions. Accordingly, the CR baseline will be updated by each additional data point to reflect the supervised person’s more typical outgoing and/or secure behavior. In some implementations, the impact of an individual CR rating on the CR baseline is at least partially dependent on a confidence level for the CR rating. As discussed above, the confidence level for the CR rating can be dependent on the number of contradictions between the evaluation data and the bioindicator data, the number of objective assessments, the number of subjective assessments, the amount of data in the RIPI data, and/or the trust score for the supervising person

Once the system has sufficient CR ratings for the supervised person, the process 900 includes evaluating the developmental status at block 912. In some implementations, the developmental status includes various components, such as physical, emotional, cognitive, social, and/or various other suitable components. The components can be based on various developmental guidelines (e.g., the WHO attachment classifications, the CDC developmental milestones, and/or guidelines from any other suitable health and/or academic institution) and can include both a current developmental status and an indication of recent and/or long terms trends in the developmental status.

In various implementations, the evaluation of the developmental status can be based on the CR baseline, average and/or weighted values in the evaluation data, whether evaluations are supported and/or contradicted by bioindicator data, whether evaluations are supported and/or contradicted by a second evaluator during each evaluation, average and/or weighted values in the bioindicator data, achieved milestones and/or classifications, expected milestones and/or classifications for the supervised person, changes in the values in the evaluation data over time, changes in the CR baseline over time, average values in the E baselines for the supervising persons providing evaluations, qualifications of the supervising persons providing evaluations (e.g., evaluations from a doctor can be weighted more heavily), additional data from one or more responsible persons (e.g., the controlling persons, doctors, and the like), proximity data between the supervised person and the supervising persons providing evaluations, context around the evaluation data (e.g., first meeting, new space, well-established relationship, known space, time of greeting, and the like), and/or various other suitable data points.

Purely by way of example, one or more of the assessments in the evaluation data can at least partially indicate which WHO attachment classification the supervised person falls into, and the assessments can be combined in a weighted average to determine which classification the supervised person falls into. For example, secure toddlers are expected to use their controlling person (e.g., their parents) and/or supervising person effectively as a base for exploration. They may or may not be distressed at the responsible person’s departure, but greet the responsible person positively when the responsible person returns, seek contact if distressed, and use the contact to settle and return to play and exploration. The assessments in the evaluation data can include objective indications on whether the toddler exhibits one or more of the behaviors above and/or subjective assessments of which WHO classification the toddler falls into. The assessments can be combined in the CR ratings, which can then be combined on a weighted basis using confidence scores for each assessment and/or the E ratings for the evaluating person.

In another specific example, the developmental assessment can include an indication of which of the CDC milestones for a given age the supervised person has achieved. In some implementations, a milestone must be indicated by multiple sets of evaluation data to be considered achieved. In some implementations, each milestone included in the developmental assessment can include a developmental score reflecting how often the supervised person has exhibited the milestone and/or how confident the process 900 is that the supervised person has achieved the milestone.

As discussed above, in some implementations, the impact of any source of data can be adjusted based on the E baseline associated with each CR rating and/or the confidence score for each CR rating. Purely by way of example, where the E baseline for a particular CR rating indicates that the evaluator is particularly harsh, a negative evaluation can be given a lower weight while a positive evaluation can be given a higher weight.

At block 914, the process 900 includes outputting the developmental status. In some implementations, the process 900 outputs the developmental status to the controlling person and/or supervising person along with any relevant breakdown of the developmental status. In such implementations, the output from process 900 enables the responsible persons to identify areas they need to focus on in providing care and supervision to encourage development of the supervised person. Further, in some implementations, the output from the process 900 includes recommendations for adjusting supervision to improve the supervised person’s development, readings related to a development area, and/or activities the responsible persons can try to target developmental areas the supervised person needs to focus on.

In some implementations, the process 900 outputs the developmental status to an accessible database (e.g., the databases 142a-142c of FIG. 2), allowing the developmental status to be used and/or shared in various ways. For example, the developmental status can be made available to a care-providing institution in an application to care-providing institution (e.g., a public and/or private school, a private care providing facility, a daycare facility, an elderly care facility, and the like). The care-providing institution can then use the developmental status in screening applications and/or in assigning the supervised person to one or more supervising persons. Purely by way of example, a school can use the developmental statuses of incoming children to try to distribute the children evenly across teachers (e.g., such that the teachers have a balance of the developmental statuses). In another specific example, a school can use the developmental statuses of incoming children to identify children for special services (e.g., to children in advanced programs, provide specialized instruction for children behind in a specific area, and the like). In some implementations sharing the developmental status, the process 900 outputs the CR baseline along with the developmental status, which can also be useful in gauging how supervised persons are likely to interact with others.

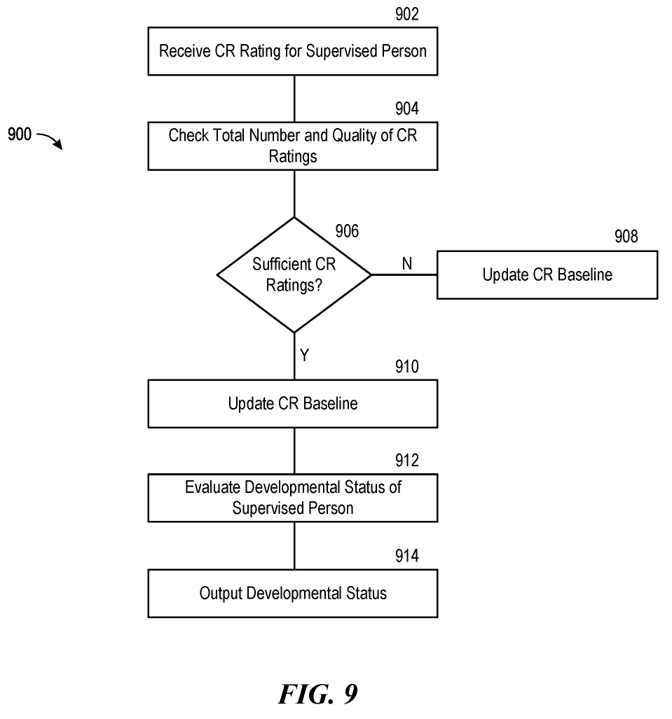

FIG. 10 is a schematic view of a process 1000 for rating a supervising person 120 after one or more rated interpersonal interactions with supervised persons 130 in accordance with some implementations of the present technology. As illustrated in FIG. 10, the supervising person 120 can have N-number of rated interpersonal interactions (RIPI-1 through RIPI-n) with one or more supervised persons 130. Each RIPI results in RIPI data (e.g., evaluation data and/or bioindicator data), which can be used to generate CR ratings, CR baselines, CG ratings, E ratings, and/or CG baselines. Each of the CR ratings, CR baselines, CG ratings, E ratings, and/or CG baselines can then be used by the remote server 140 to generate a RIPI score for the supervising person 120. As discussed above, the RIPI score can indicate the impact that the supervising person 120 is having on the supervised persons 130 that they interact with. Details on the generation of the CG ratings, E ratings, CG baselines, and the RIPI score are discussed in more detail with respect to FIGS. 11 and 12 below.

FIG. 11 is a flow diagram of a process 1100 for rating a supervising person after a single interpersonal interaction in accordance with some implementations of the present technology. The process 1100 can be at least partially executed by a module on the cloud sever 140 described above with respect to FIG. 2 to process and evaluate interactions between persons in the system. As illustrated in FIG. 11, the process 1100 is generally similar to the process 800 discussed above with respect to FIG. 8, but is used to generate CG score for a supervising person based on an interpersonal interaction.

In the illustrated implementation, the process 1100 begins at block 1102 by detecting an interpersonal interaction (and/or a set of interpersonal interactions) between the supervising person and a supervised person. As discussed above, the interaction can be detected by an indication from the supervising person, location and/or proximity data from the supervising person and/or the supervised person, an indication from the controlling person (or another relevant responsible person), at a reoccurring time (e.g., after a scheduled drop-off (e.g., morning drop off), a schedule takeover (e.g. when one care provider goes on break), and the like) and/or any other suitable detection means.

At block 1104, the process 1100 includes receiving bioindicator data from the supervised person. The bioindicator data can be received through the responsible person (e.g., when the wearable device communicates the bioindicator data using the shortrange communication component) and/or through a network connection. In some implementations, the bioindicator data includes periodic updates on the bioindicators of the supervised person throughout the detected interpersonal interaction. In some implementations, the bioindicator data includes a continuous record of the bioindicators of the supervised person throughout the detected interpersonal interaction.

At block 1106, the process 1100 includes receiving an evaluation of the supervised person. As discussed above, the evaluation can include various objective and/or subjective assessments of the supervised person’s behavior during the detected interaction. Further, the evaluation can include evaluation data from one or more responsible persons associated with the supervising person and/or the supervised person.

At block 1108, the process 1100 includes checking the bioindicators of the supervised person for contradictions (or corroborations) with the data in the evaluation. Purely by way of example, the evaluation can include an assessment of the supervised person’s stress levels during the interaction (e.g., whether a child was calm or stressed during separation from their parent), which can be at least partially contradicted and/or corroborated by the bioindicators of the supervised person. Additionally, or alternatively, the process 1100 can include checking the evaluation data from multiple responsible persons associated with the supervising person and/or the supervised person for contradictions with the evaluation data from the supervised person.

At decision block 1110, if a contradiction was found, the process 1100 can continue to block 1112 to address the contradiction, else the process 1100 can continue to block 1114.

At block 1112, the process 1100 includes prompting one or more supervising persons for a reevaluation of the interaction. In some implementations, the prompt includes an indication of the detected contradiction to help direct the supervising person’s attention to what assessments to revisit. In some implementations, the process 1100 then returns to blocks 1106-1110 to receive the reevaluation of the supervised person and check for remaining contradictions between the reevaluation and the bioindicators.

In some implementations, the process 1100 can record the number of contradictions between an evaluation and the bioindicators, the type of contradictions (e.g., that all the contradictions were related to a category of assessment, objective vs. subjective assessments, and the like), the severity of the of contradiction (e.g., whether the contradiction suggested the evaluation was slightly inaccurate or completely wrong) on each lap through block 1112, the number of iterations the process 1100 must follow between blocks 1106 and 1112 before the contradictions are resolved, the number of contradictions (or corroborations) between an evaluation from a first supervising person and an evaluation from a second supervising person, the relative rank and/or qualifications between the first and second supervising persons, and/or various other metrics. Each of the metrics for the contradiction detections can be recorded with the evaluation of the interaction and/or used when evaluating the supervising person (e.g., in block 1116 discussed below).

At block 1114, the process 1100 includes retrieving the CR baseline for the supervised person. The CR baseline can help the process evaluate how the assessments in the evaluation data compare to what might be expected for the supervised person. In various implementations, the CR baseline can be retrieved from one of the databases 142a-142c (FIG. 2) in the remote server 140, a storage device on the wearable device 132 (FIG. 1), and/or from one or more of the responsible persons.

At block 1116, the process 1100 includes generating a CG rating and/or an E rating for the supervising person based on the evaluation data, the contradiction metrics, and/or the CR baseline.

Purely by way of example, the CG rating and/or the E rating can be calculated using a weighted polynomial equation. In a specific, non-limiting example, similar to the example discussed above, the weighted polynomial equation can have a term for each of the CDC milestones for an age of the supervised person. The weight assigned to each of the CDC milestones can be partially dependent on the evaluation data (e.g., the evaluation data can include a “not applicable” response for one or more milestones that adjusts a weight assigned to a milestone to zero; the weights for each milestone can be dependent on a total number of milestones being considered, and the like). Additionally, or alternatively, the weight assigned to each of the CDC milestones can be partially dependent on the CR baseline for the supervised person (e.g., thereby accounting for whether a supervised person normally exhibits various CDC milestones). The CG rating can reflect whether the supervising person is accurately evaluating supervised persons based on the contradiction metrics and/or how the current evaluation compares to the CR baseline. For example, a large number of contradictions and/or relatively strong contradictions from the bioindicator data can suggest that the supervising person is not accurately evaluating supervised persons. In another example, an evaluation that dramatically departs from the CR baseline can suggest that the evaluation is not accurate, even if not contradicted by the bioindicator data. An inaccurate evaluation from the supervising person can result in a worse CG rating.

Additionally, or alternatively, the CG rating can reflect how the supervising person impacts the supervised person in the current interpersonal interaction. Purely by way of example, an evaluation with assessments that the supervised person was more secure than their CR baseline during an interaction (if not contradicted by the bioindicator data and/or large enough to suggest an error) can suggest that the supervising person had a positive impact on the supervised person. Accordingly, improvements over the CR baseline can result in a better CG rating. In another example, an evaluation with assessments that the supervised person had a more positive mood than their CR baseline during an interaction (if not contradicted by the bioindicator data and/or large enough to suggest an error) can suggest that the supervising person had a positive impact on the supervised person.

The E rating can help account for nuances in how various supervising persons evaluate the supervised person. For example, where the evaluation indicates that the supervising person assessed the supervised person below where the process 1100 expects based on their CR baselines, the E rating can provide an adjustment factor to correct towards the CR baseline. The correction can be helpful in processing later evaluations from the supervising person in order to accurately generate CR ratings and/or to assess the developmental status of the supervised person.

In some implementations, the process 1100 associates the CG rating with a confidence level. The confidence level reflects how likely the CG rating is to be accurate based on the data that is used to generate the CG rating and/or the persons involved. In various implementations, the confidence level can be at least partially dependent on a confidence level for the current CR baseline, the amount and/or quality of the contradictions between the evaluation data and the bioindicator data, the number of assessments in the evaluation data, and/or a various other factors. For example, in general, the less data that is available to generate the CG rating (e.g., from fewer assessments in the evaluation data), the lower the confidence level the process 1100 will assign to the CG rating. The confidence level for CR baseline can also be low, for example, when the supervised person does not have a well-established CR baseline. In a specific example, when the supervised person is new to the system, the system may not be able to establish a CG rating with much confidence since the system will have little (or no) data to compare the interaction against. Accordingly, even if the evaluation indicates a negative (e.g., a very stand-offish) interaction, the system cannot determine how much causal impact to assign to the supervising person vs. the supervised person.

At block 1118, the process 1100 includes outputting the CG rating. The output can be stored to one of the databases 142a-142c (FIG. 2) in the remote server 140, used in one or more additional modules on the remote server 140 and/or additional processes in the system, and/or shared with one or more responsible persons (e.g., shared with the supervising person that is rated, shared with the controlling person associated with the supervised person, and the like). In some implementations, the output at block 1118 can trigger the process 1200 described below with respect to FIG. 12.

FIG. 12 is a flow diagram of a process 1200 for generating a RIPI score for a supervising person after multiple interpersonal interactions in accordance with some implementations of the present technology. The process 1200 can be at least partially executed by a module on the cloud sever 140 described above with respect to FIG. 2 to evaluate supervising persons in the system.

In the illustrated implementation, the process 1200 begins at block 1202 by receiving the CG rating. In some implementations, the CG rating is received directly from the process 1100 of FIG. 11. In some implementations, the CG rating is received from a database (e.g., one of the databases 142a-142c (FIG. 2) in the remote server 140) when a responsible person prompts the system to execute the process 1200.

At block 1204, the process 1200 includes checking the total number and quality of CG ratings. The quality of the CG rating can be at least partially dependent on a confidence level associated with the CG rating.

At block decision block 1206, if there are a sufficient number and/or quality of CG ratings already received for the supervised person, the process 1200 continues to block 1210; else the process 1200 continues to block 1208.

At block 1208, the process 1200 includes updating the CG baseline for the supervising person. Like the CR baseline, the CG baseline can be used in processing the RIPI data to adjust expectations for the interactions and/or the evaluations from the interactions. Purely by way of example, as discussed above, the CG baseline can be factored into the evaluation of the developmental status of the supervised person in block 912 of FIG. 9. As discussed in more detail below, the CG baseline can also help the process 1200 efficiently generate a RIPI score for the supervising person by keeping a running and/or weighted record of their impacts on interaction before they have had sufficient CG ratings to generate the RIPI score.

At block 1210, the process 1200 includes generating (or updating) the RIPI score for the supervising person and updating the CG baseline for the supervising person. Purely by way of example, the RIPI score can be calculated using a weighted polynomial equation. In a specific, non-limiting example, the weighted polynomial equation can have a term for each of the CG Ratings for the supervising person. In this specific example, the weight assigned to each of the CG Ratings can be based at least in part on a confidence score for each CG Rating, E Ratings and/or CR Baselines associated with each CG Rating, and the like. As discussed above, the RIPI score provides an indication of the impact the supervising person 120 has on the supervised persons under their responsibility over time. Accordingly, the RIPI score can be generated based on the CG baseline, average and/or weighted values in the evaluation data, average and/or weighted values for the contradiction metrics, whether evaluations are supported and/or contradicted by a second evaluator during each evaluation, changes in the values in the evaluation data for one or more supervised persons over time, changes in the CR baseline for one or more supervised persons over time, changes in the developmental status of one or more supervised persons over time, reviews from one or more responsible persons (e.g., the controlling persons), proximity data between the supervised person and the supervising persons providing evaluations, context around the evaluation data (e.g., first meeting, new space, well-established relationship, known space, time of greeting, and the like), and/or various other suitable data points.

Purely by way of example, each of the assessments in the evaluation can have an expected distribution for a set of supervised persons, and the RIPI score can be at least partially based on how the supervised persons under the responsibility of the supervising person compare to the expected distribution. For example, as discussed above for the WHO attachment classifications, about 55% of toddlers are expected to be classified as secure; about 20% are expected to be classified as insecure-avoidant; about 15% are expected to be classified as insecure-ambivalent, anxious or resistant; and up to 8% are expected to be classified as disorganized-disoriented. Accordingly, if the evaluation data indicated that about 70% of the toddlers the supervising person evaluated are secure (after quantity and quality controls), the above average distribution can be reflected positively in the RIPI score for the supervising person. Conversely, if the evaluation data indicated that about 35% of the toddlers the supervising person evaluated are secure (after quantity and quality controls), the below-average distribution can be reflected negatively in the RIPI score for the supervising person.

In some implementations, the impact of any source of data can be adjusted based on the CR baselines and/or developmental status for the supervised persons that the supervising person is responsible for. Purely by way of example, where the CR baselines and/or developmental status indicate that one or more the supervised persons were behind on development before any interactions with the supervising person, the impact of their evaluations and/or developmental status can have a lower weight in determining the RIPI score. In some such implementations, the changes in the CR baselines and/or developmental status for the one or more supervised persons can be given more weight in determining the RIPI score. Returning to the example above, if the changes in the CR baselines and/or developmental status indicate that the supervised persons accelerated in development after interactions with the supervising person, that acceleration can be weighted more heavily than their current CR baselines and/or developmental status.

At block 1212, the process 1200 includes outputting the RIPI score. In some implementations, the process 1200 outputs the RIPI score to the supervising person along with any relevant breakdown of their score. In such implementations, the output from process 1200 enables the supervising person to identify areas they need to focus on in providing care and supervision. Further, in some implementations, the output from the process 1200 includes recommendations on training exercises, readings, and/or activities the supervising person can try to improve their impact on the persons under their supervision.

In some implementations, the process 1200 outputs the RIPI score to the controlling person(s) associated with the supervised persons associated with the supervising person, one or more other supervising persons (e.g., a head childcare provider can be supplied with the RIPI scores for each of their workers), and/or to an accessible database (e.g., publicly accessible, accessible to controlling persons 110 registered in the system 100 (FIG. 1), and the like). The availability of the RIPI score can allow responsible persons to make more informed decisions about the supervising persons they entrust with the responsibility over one or more supervised persons. Alternatively, or additionally, the availability of the RIPI score can allow the responsible persons to more closely monitor the development of one or more supervised persons in areas that the RIPI score identifies as weaknesses for the supervising person.

### Example Systems and Methods of AI/ML Processes According to Implementations of the Present Technology

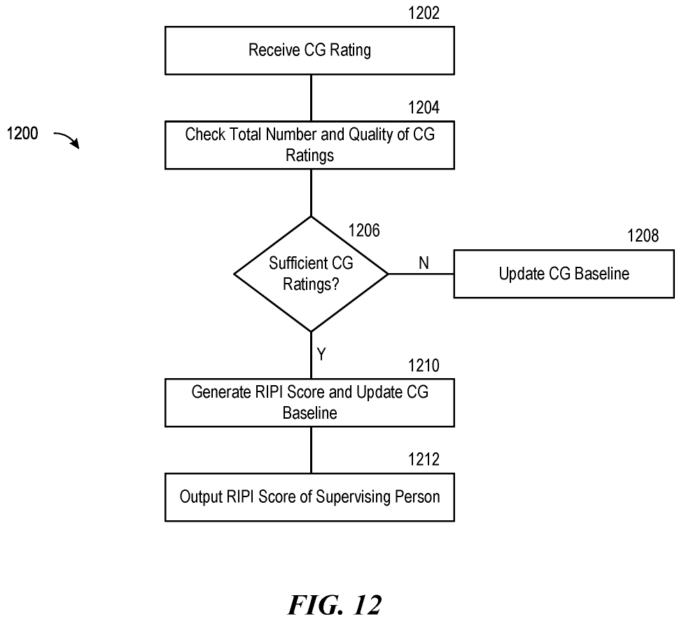

FIG. 13 is a schematic view of a subsystem 1300 for making recommendations regarding the developmental status of a supervised person in accordance with some implementations of the present technology. The subsystem 1300 includes one or more controlling persons 110 (one shown), the remote server 140, and one or more third parties 180 (one shown). As illustrated in FIG. 13, the database 142 on the remote server 140 maintains a record of the developmental data, bioindicator data, and/or other data for one or more supervised persons. The data on the database 142 can be sorted, linked, classified, and/or labelled in any suitable manner, allowing the data to be easily communicated to the third party 180 as research data. The research institution can then store the research data on the database 1382. In some implementations, the database 1382 maintains any sorting, linking, classifications, and/or labeling in the research data. In some implementations, the database 1382 redoes (or implements for the first time) any sorting, linking, classifications, and/or labeling of the research data.

The third party 180 can then input the research data into one or more algorithms 1384 to generate a report on the developmental status of a supervised person and/or various recommendations regarding the developmental status of a supervised person associated with the research data. The algorithm(s) 1384 can include AI/ML algorithms, predictive models previously generated by the AI/ML algorithms, predictive models built by the third party 180 (e.g., based on their research into human development), and/or any algorithm.

In some implementations, the recommendations include broad recommendations based on the developmental status and general trends for supervised persons identified by the third party 180 (e.g., that children with a certain amount of reading time per day are associated with positive cognitive development, that elderly persons with a certain amount of exercise maintain cognitive function longer, and the like). In some implementations, the recommendations include specific changes to the daily life of the supervised person based on the developmental status, general trends, and/or trends specific to the supervised person (e.g., identifying that a particular child responds especially well to a particular cognitive exercise; that a particular child responds well (or poorly) to time with a particular care provider; that a particular care provider is impacting a particular child’s development in a certain way; that a particular child needs additional amounts of a nutrient; and the like). The developmental status of a supervised person and/or various recommendations can then be communicated back to the remote server 140 and stored in the database 142.

The controlling person 110 can then use the subsystem 300 on the electronic device 112 to access the remote server 140 and the assessed developmental status and/or recommendations. In some implementations, the remote server 140 prompts the controlling person 110 when the developmental status and/or recommendations are received. In some implementations, the developmental status and/or recommendations are responsive to prompts from the controlling person 110 to send the research data to one or more third parties 180. Purely by way of example, the controlling person can prompt the remote server 140 to send the research data to an institution known for their ability to accurate assess the physical development of a supervised person and recommend effective changes for improving the same. The remote server 140 can then send the research data to the institution and make the assessed developmental status and/or recommendations available to the controlling person 110 once received.

As further illustrated in FIG. 13, the controlling person 110 can provide feedback regarding the assessed developmental status and/or recommendations to the remote server 140. Purely by way of example, the feedback can include indications of the success of the recommendations and/or updates on the supervised person after implementing the recommendations, the controlling person’s difficulty in implementing the recommendations, any changes the controlling person 110 made while implementing the recommendations, suggested changes to the recommendations, and/or any other suitable feedback. The remote server 140 can then forward the feedback to the third parties 180 to allow them to use the feedback in updating the algorithms 1384.

It will be understood that although the discussion of the subsystem 1300 herein included one or more controlling person(s) 110, the subsystem can additionally (or alternatively) include one or more supervising persons 120 connected to the subsystem 1300. For example, the supervising person(s) 120 can also access the remote server 140 to view the assessed developmental status and/or recommendations; and/or to provide feedback regarding the assessed developmental status and/or recommendations to the remote server 140.

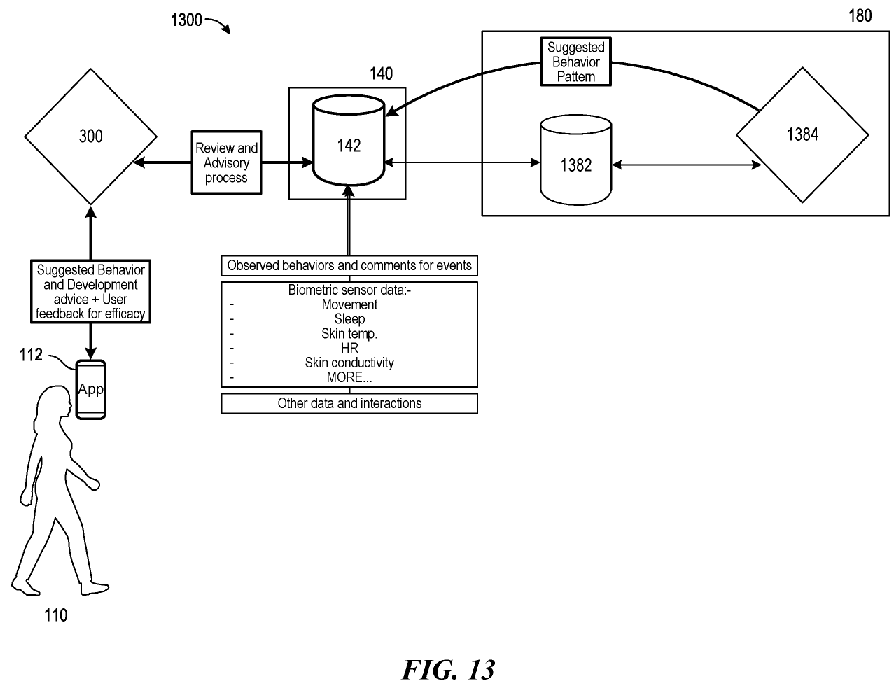

FIG. 14 is a schematic view of a subsystem 1400 for developing a predictive model for monitoring the developmental status of supervised persons 1430 in accordance with some implementations of the present technology. The subsystem 1400 includes one or more supervising persons 120 (one shown), one or more supervised persons 1430 (three shown, referred to individually as first-third supervised persons 1430a-1430c), and the remote server 140. As illustrated in FIG. 14, the supervising person(s) 120 can provide developmental data (e.g., observed behavior, overserved milestones, evaluations, information about specific events (e.g., stress events), timing of observed behavior and/or milestones, and the like) for each of the supervised persons 1430 to the remote server 140. Meanwhile, the wearable sensors on each of the supervised persons 1430 can provide bioindicator data to the remote server 140. The remote server 140 can sort, link, classify, and/or label the data in any suitable manner into the database 142, allowing the data to be easily accessed and used by an AI/ML component 1442 on the remote server 140.

In the implementation illustrated in FIG. 14, the AI/ML component 1442 includes various system preferences associated with the health and development of the supervised persons 1430 (e.g., recommended sleep, recommended hydration, and the like), as well as a freely learning AI/ML algorithm that receives the data, identifies patterns in the data, and/or generates a predictive model. The predictive model can be used to assess the developmental status of any of the first-third supervised persons 1430a-1430c, assess the developmental status and/or wellness of the supervised persons 1430 as a group (e.g., to identify when a group or subgroup of the supervised persons 1430 deviate from an expected developmental status), and/or to identify general trends in human development. For example, the supervising person(s) 120 can access the predictive model to assess the developmental status of each of the supervised persons 1430 to track their development over time. In another example, the supervising person(s) 120 can access the predictive model to identify general trends to update and/or maintain various activities (e.g., the supervising person(s) 120 can see that a change in their supervision is impacting the development of the supervising person(s) 120 and maintain or reverse the change accordingly).

It will be understood that although the discussion of the subsystem 1400 herein included one or more supervising person(s) 120 controlling person(s) 110, the subsystem can additionally (or alternatively) include one or more controlling persons 110 (FIG. 1) connected to the subsystem 1400. For example, the controlling person(s) 110 can also upload developmental data on one or more of the supervised persons 1430 and/or access the remote server 140 to access the predictive models.

FIG. 15 is a flow diagram of a process 1500 for associatively linking data from one or more subsystems in accordance with some implementations of the present technology. The process 1500 can be at least partially executed by a module on the remote server 140 described above with respect to FIG. 2 to process the data from one or more subsystems in the system.

In the illustrated implementation, the process 1500 begins at block 1502 when any target data is received. The target data can be received from any of the responsible persons (e.g., uploading developmental data and/or relaying an update from the wearable sensors), from a wearable sensor, and/or from any other suitable source (e.g., from another database, from a medical professional evaluating the supervised person, and the like).

At block 1504, the process 1500 includes formatting the data for storage, communication, and/or later use. In some implementations, the process 1500 converts the target data into a standardized formatting at block 1504. In some implementations, the process 1500 processes the target data while converting the target data into the standardized formatting. For example, the process 1500 can include processing data from a skin conductivity sensor to identify cognitive activity, emotional statuses, and/or stress levels and output the identified bioindicator data (e.g., rather than storing the raw skin conductivity data). In another example, the process 1500 can parse natural language evaluations of the supervised person for the relevant developmental data.

At block 1506, the process 1500 includes classifying and labelling the target data for later use by an AI/ML algorithm, predictive model, and/or research institution. As used herein, classifying refers to the process of creating classes for the target data (or sub-parts of the target data) that contain numerous properties, including additional subclasses, while labeling refers the process of sorting the target data into the created classes. Purely by way of example, classifying the target data can create a developmental data class and a bioindicator data class, each of which can have subclasses. Purely by way of another example, the developmental data may be divided into objective and subjective subclasses classes, each of which can have additional subclasses. In the examples above, after target data is received, the process 1500 can then label the target data (or portions thereof) as ‘developmental data’ or ‘bioindicator data,’ then label any developmental data as ‘objective data’ or ‘subjective data.’

At block 1508, the process 1500 includes checking for associated data. In some implementations, the process 1500 checks the received target data for associations between various parts of the received target data. For example, the process 1500 can check for an association between any developmental data and any bioindicator data. Purely by way of example, an evaluation of the stress levels of a supervised person in the developmental data can be associated with various bioindicators (e.g., heart rate, blood pressure, skin conductivity, and the like). In some implementations, the process 1500 checks the received target data for associations with other data, such as previously received target data. For example, the process 1500 can check for an association between evaluations from multiple responsible persons about the same event; check for an association between evaluations of a recurring event (e.g., an evaluation of the supervised person after a daily activity); and/or check for any other suitable associations.

At decision block 1510, if any associated data is found, the process 1500 continues to block 1514 to link the data; else the process 1500 continues to block 1512.

At block 1512, the process 1500 includes storing the processed target data for later use. In some implementations, the processed target data is stored in the database 142 of the remote server 140 (FIG. 1). In some implementations, the processed target data is sent to the database 182 of the third party 180 (FIG. 1) for storage. In some implementations, the process 1500 includes randomly storing the target data in one of: a training data database, a validation data database, and a testing data database. As discussed in more detail below, the training data database can later be used by an AI/ML algorithm to generate a predictive model; the validation data can be used to assess the validity of the predictive model as it is generated and/or for early stopping when an error on the validation data increases; and the test data can be used to provide an unbiased evaluation of the predictive model on the training data.

At block 1514, the process 1500 includes creating a link between the associated data. The link allows a human or computer process studying the target data to quickly view associated data to check for corroborations (or contradictions) between the data (e.g., check whether the bioindicators corroborate (or contradict) the developmental data); develop a more complete picture (e.g., supplementing the developmental data from a first responsible person with the developmental data from a second responsible person); and/or view the evolution of the target data over time.

At block 1516, the process 1500 includes storing the processed target data with the generated links for later use. As discussed above, in various implementations, the processed target data can be stored in the database 142 of the remote server 140 (FIG. 1) and/or sent to the database 182 of the third party 180 (FIG. 1) for storage. Further, in some implementations, the process 1500 includes randomly storing the target data in one of: a training data database and a validation data database. As discussed in more detail below, the training data database can later be used by an AI/ML algorithm to generate a predictive model while the validation data can be used to assess the validity of the predictive model once generated.

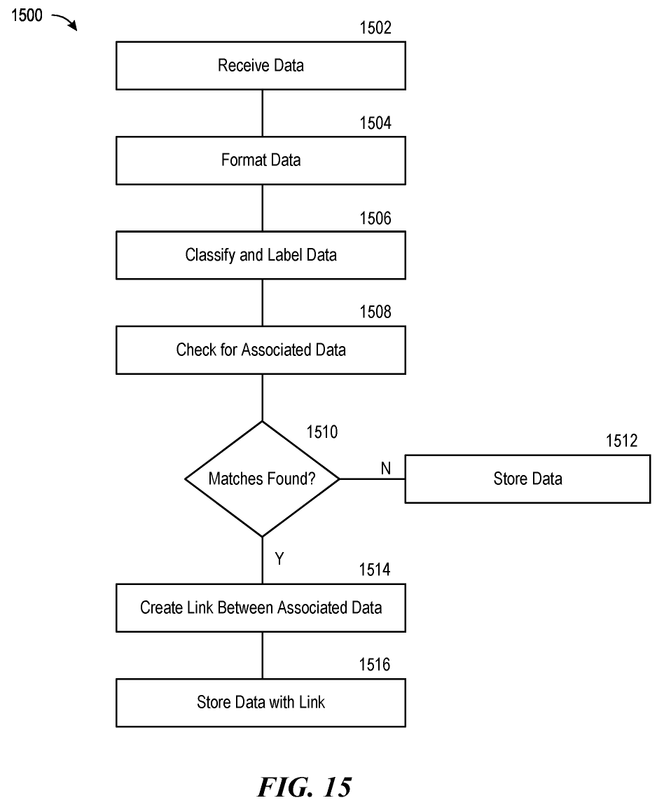

FIG. 16 is a flow diagram of a process 1600 for adapting a predictive model for a specific supervised person and applying the adapted predictive model in accordance with some implantations of the present technology. Like the process 1500 discussed above with respect to FIG. 15, the process 1600 can be at least partially executed by a module on the remote server 140 described above with respect to FIG. 2.

In the illustrated implementation, the process 1600 begins at block 1602 with retrieving target data for the specific supervised person to use as training data to adapt a predictive model to the specific supervised person. The training data can include a history of the target data for the specific supervised person stored in the remote server 140 (e.g., the target data received in the last week, in the last month, the last six months, the last year, and/or any other suitable time period) and/or target data from a plurality of supervised persons identified as similar to the specific supervised person.

At block 1604, the process 1600 applies an AI/ML algorithm with a baseline predictive model to the target data from block 1602 to generate an adapted predictive model. The baseline predictive model can be from a previous generation for the specific supervised person, other modules in the system (e.g., from the process 1700 discussed below with respect to FIG. 17), a research institution or other regulatory institution (e.g., the CDC or the WHO), and/or from any other suitable source. In various implementations, the AI/ML algorithms can include, but are not limited to, one or more of: case-based reasoning, rule-based systems, artificial neural networks, convoluted neural networks (CNN), decision trees, support vector machines, regression analysis, Bayesian networks (e.g., naive Bayes classifiers), genetic algorithms, cellular automata, fuzzy logic systems, multi-agent systems, swarm intelligence, data mining, machine learning (e.g., supervised learning (e.g., gradient descent or stochastic gradient descent), unsupervised learning, reinforcement learning), and hybrid systems. Details on the process for generating and/or updating a predictive model are discussed below with respect to FIG. 17.

At block 1606, the process 1600 outputs the adapted predictive model. As discussed above, the adapted predictive model can be used to more accurately evaluate any new target data to identify a current developmental status for the specific supervised person, predict the impact various changes will have on the developmental status for the specific supervised person, and/or generate recommendations for changes to intentionally impact the developmental status for the specific supervised person in a desired way.

At block 1608, the process 1600 receiving and/or retrieves recent target data for the specific supervised person. At block 1610, the process 1600 applies the adapted predictive model to the recent target data, thereby generating an accurate assessment of the current developmental status of the supervised person, a prediction of the trends in the developmental status of the supervised person, and/or a prediction in how one or more changes will impact the developmental status of the supervised person.

At block 1612, the process 1600 outputs the result from block 1612. In some implementations, the process 1600 outputs the result straight to one or more responsible persons. For example, the process 1600 can be triggered by a request from a controlling person (e.g., a parent requesting an evaluation of their child), and the result from block 1612 can be sent directly to the controlling person. In some implementations, the process 1600 outputs the result to the remote server 140 (FIG. 1), where the result can be saved and later accessed by one or more responsible persons.

FIG. 17 is a flow diagram of a process 1700 for training a predictive model to predict assess current developmental statuses of supervised persons and/or predict the impact of one or more changes on the developmental statuses of supervised persons in accordance with some implementations of the present technology. Like the processes 1500 and 1600 discussed above with respect to FIGS. 15 and 16, the process 1700 can be at least partially executed by a module on the remote server 140 described above with respect to FIG. 2.

In the illustrated implementation, the process 1700 begins at block 1702 by receiving target data for N-number of supervised persons. The number N can be any number of supervised persons connected to the system 100 (FIG. 1), such as one, two, five, ten, fifty, one hundred, one thousand, or any other suitable number of supervised persons.

At blocks 1704-1708, the process 1700 loops through the target data for each of the N-number of supervised persons. At block 1706, the process 1700 includes classifying and labelling the target data. As discussed above with respect to FIG. 15, classifying the target data includes determining the classes for the target data, while labelling the target data includes sorting target data into the classes.

At block 1710, the process 1700 includes aggregating data in each of the classes. Purely by of example, the process 1700 can aggregate all of the developmental data related to a particular type of evaluation (e.g., an evaluation of cognitive development), as well as any linked and/or associated data.

At block 1712, the process 1700 includes sorting the data into sets. The sets can include one or more training sets that can be used in block 1714, one or more validation sets that can be used in block 1718, one or more testing data sets, and/or any other suitable sets.

At block 1714, the process 1700 applies an AI/ML algorithm to the target data. As discussed above, the AI/ML algorithm can include can include, but is not limited to, case-based reasoning, rule-based systems, artificial neural networks, decision trees, support vector machines, regression analysis, Bayesian networks (e.g., naive Bayes classifiers), genetic algorithms, cellular automata, fuzzy logic systems, multi-agent systems, swarm intelligence, data mining, machine learning (e.g., supervised learning (e.g., gradient descent or stochastic gradient descent), unsupervised learning, reinforcement learning), and hybrid systems.

Purely by way of example, the AI/ML algorithm can be a supervised ML algorithm (e.g., a neural network or a naive Bayes classifier) that can be trained on the training data set using a supervised learning method (e.g., gradient descent or stochastic gradient descent). The training data set can include pairs of generated “input vectors” with the associated corresponding “answer vector” (commonly denoted as the target). The ML algorithm generates (or starts with) a basic predictive model and runs the basic predictive model with the training data set and produces a result, which is then compared with the target, for each input vector in the training data set. Based on the result of the comparison and the specific ML algorithm being used, the parameters of the model can be adjusted and/or deleted, and/or additional parameters can be created. That is, the ML algorithm can include both variable selection and parameter adjustment. The process is then repeated with the updated models to gradually fit the model to the training data set. Once the ML algorithm is satisfied with the generated predictive model, the process 1700 moves to block 1716.

The predictive model can then be used to predict the responses for the observations in the validation data set. The validation data set can provide an unbiased evaluation of a model fit on the training data set while tuning the model parameters. The validation data sets can also, or alternatively, be used for regularization by early stopping, (e.g., by stopping the AI/ML algorithm when an error in a prediction on the validation data set increases by more than a predetermined threshold, which can be a sign of overfitting). In some implementations, the error of predictive model applied to the validation data set can fluctuate during training, such that ad-hoc rules may be used to decide when overfitting has truly begun.

At block 1716, the process 1700 includes outputting the generated predictive model for a final check through the testing data set. At block 1718, the process 1700 includes checking the predictive model against the testing data to provide an unbiased evaluation of the final predictive model. Importantly, the error (or lack thereof) in a prediction on the testing data set is never used to select variables and/or adjust parameters in the predictive model, only to assess the accuracy of the predictive model.

In some implementations, if the accuracy of the predictive model on the testing data is not satisfactory, the process 1700 returns to block 1714 to apply the AI/ML algorithm to a new training data set (or reapply the AI/ML algorithm to the previous training data set).

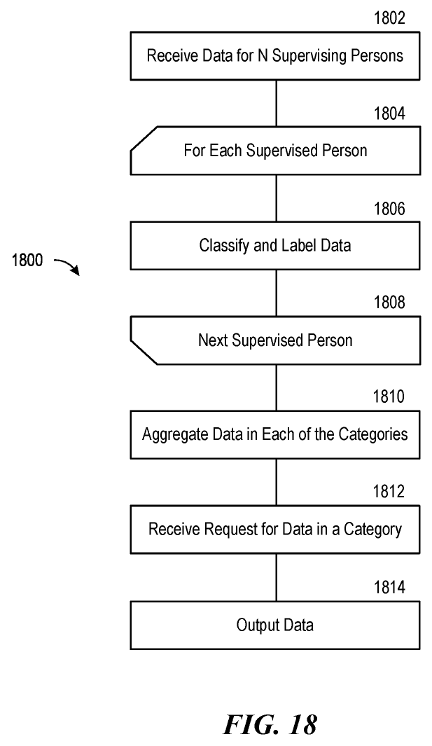

At block 1720, the process 1700 outputs the predictive model for use elsewhere in the system 100 (FIG. 1). Purely by way of example, the output predictive model can be used in block 1904 of FIG. 19, discussed in more detail below.

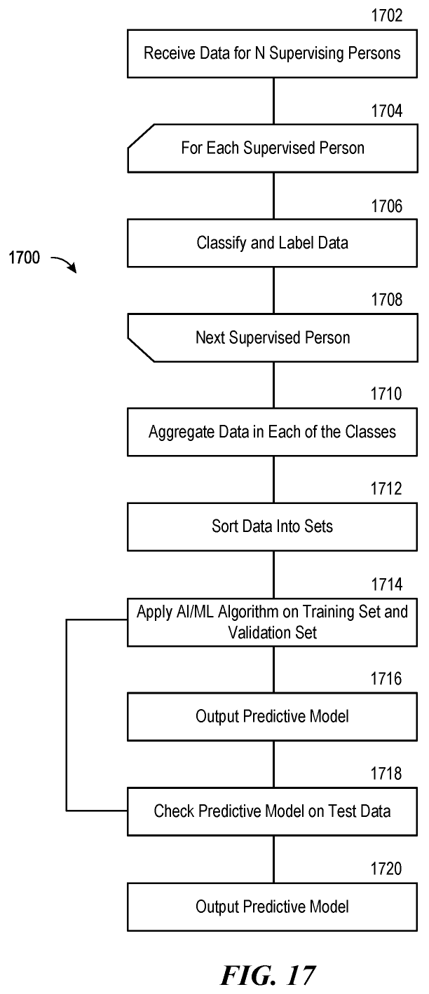

FIG. 18 is a flow diagram of a process 1800 for aggregating and outputting data for use in researching human development in accordance with some implementations of the present technology. Like the processes discussed above with respect to FIGS. 15-17 the process 1800 can be at least partially executed by a module on the remote server 140 described above with respect to FIG. 2.

In the illustrated implementation, the process 1800 begins at block 1802 by receiving target data for N-number of supervised persons. As discussed above, the number N can be any number of supervised persons connected to the system 100 (FIG. 1), such as one, two, five, ten, fifty, one hundred, one thousand, or any other suitable number of supervised persons.

At blocks 1804-1808, the process 1800 loops through the target data 1700for each of the N-number of supervised persons. At block 1806, the process 1700 1800 includes classifying and labelling the target data. As discussed above with respect to FIG. 15, classifying the target data includes determining the classes for the target data, while labelling the target data includes sorting target data into the classes.

At block 1810, the process 1800 includes aggregating data in each of the classes. Purely by of example, the process 1800 can aggregate all of the developmental data related to a particular type of evaluation (e.g., an evaluation of cognitive development), as well as any linked and/or associated data.

At block 1812, the process 1800 includes receiving a request for target data in one or more classifications. Purely by way of example, request can come from a third party 180 (FIG. 1) interested in a particular aspect of human development. In another example, the request can come from one or more regulatory organizations interested in a survey of the current status of human development and/or in studying a particular aspect of human development.

At block 1814, the process 1800 includes outputting the requested data to the requesting party. Because the process already labeled and aggregated the target data, the output can be achieved efficiently, allowing the system 100 (FIG. 1) to increase access to relevant target data.

FIG. 19 is a flow diagram of a process 1900 for using a predictive model to the target data of a supervised person in accordance with some implementations of the present technology. Like the processes discussed above with respect to FIGS. 15-18 the process 1900 can be at least partially executed by a module on the remote server 140 described above with respect to FIG. 2.

In the illustrated implementation, the process 1900 begins at block 1902 by receiving a request from a responsible person. The request can indicate a specific application of the predictive model, such as to check the developmental status of a supervised person, assess the impact one or more changes that the responsible person is considering will have on developmental status, generate recommendations for changes to intentionally impact the developmental status, and the like.

At block 1904, the process 1900 includes retrieving a relevant predictive model. In some implementations, the process 1900 retrieves the predictive model from the database 142 (FIG. 1) on the remote server 140 (e.g., after being output at block 1720 of FIG. 17)). In some implementations, the process 1900 retrieves the predictive model from a third party 180 (e.g., after being output from the algorithms 1384 of FIG. 13) and/or any other suitable institution (e.g., the CDC and/or the WHO). In some implementations, which predictive model the process 1900 retrieves is dependent on the request received at block 1902. For example, a first predictive model may be better at assessing a current developmental status of a supervised person while a second predictive model may be better at predicting the impact one or more changes will have on the developmental status. In another example, a first predictive model may be better at assessing a first aspect of the current developmental status (e.g., cognitive development) while a second predictive model may be better at assessing a second aspect of the current developmental status (e.g., social development). In some implementations, the process 1900 automatically determines which predictive model to retrieve based on the request received at block 1902. In some implementations, the request includes an indication of which predictive model to retrieve.

At block 1906, the process 1900 includes applying the predictive model to the target data for the supervised person. In some implementations, the process 1900 applies the predictive model only to recent target data (e.g., the most recently received target data, the target data for the last few days, last week, last month, and the like). The application to recent target data can efficiently generate an analysis of the current developmental status of the supervised person. In some implementations, the process 1900 applies the predictive model to all of the target data for the supervised person within the system 100 (FIG. 1). The broader application can be useful when assessing the impact one or more changes will have on the developmental status and/or when generating recommendations for changes.

At block 1908, the process 1900 includes outputting a result of the application of the predictive model. Depending on which predictive model was applied, the result can be an estimate of the current developmental status, the assessment of the impact of one or more changes, one or more recommendations for changes, and the like. In some implementations, the process 1900 outputs the result directly to the responsible person that submitted the request. In some implementations, the process 1900 outputs the result to the remote server 140 (FIG. 2), allowing any responsible person with access permissions to view the result (e.g., allowing both a controlling person and a supervising person to view the result).

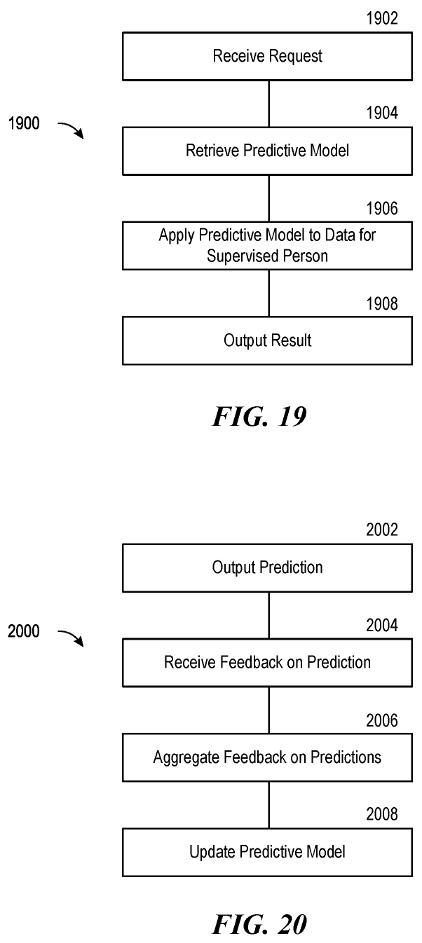

FIG. 20 is a flow diagram of a process 2000 for updating a predictive model in accordance with some implementations of the present technology. Like the processes discussed above with respect to FIGS. 15-19 the process 2000 can be at least partially executed by a module on the remote server 140 described above with respect to FIG. 2.

In the illustrated implementation, the process 2000 begins at block 2002 by outputting a prediction from a predictive model. The prediction can include an assessment of how one or more changes will impact the developmental status of the supervised person, one or more recommendations for changes to intentionally impact the developmental status of the supervised person, one or more predictions on how the supervised person will react near-term to the predictions, and the like. In some implementations, the output at block 2002 is the same as the output at block 1908 discussed above with respect to FIG. 19. As discussed above, the process 1900 can output the result directly to a specific responsible person and/or to the remote server 140 (FIG. 2) to allow multiple responsible persons to view the prediction. In some implementations, the prediction is a prediction of a broad correlation in human development (e.g., people who exercise within X-hours of waking up are more attentive throughout the day and therefore accelerate their physical, emotional, cognitive, and/or social development). The process 2000 can output said broad predictions to the remote server 140 (FIG. 2) to allow multiple responsible persons and/or third parties 180 to view the prediction.

At block 2004, the process 2000 includes receiving feedback on the prediction from one or more responsible persons. The feedback can include one or more indications of the accuracy of the prediction (e.g., whether the prediction correctly indicated how a supervised person would react to a change near-term and/or in their long-term development); one or more indications of the feasibility of any recommendations for changes (e.g., when a recommended change cannot possibly be made, when a change is easy to make, and the like); one or more indications of unexpected results (e.g., unexpected reactions to a change); one or more additional changes that the responsible person made (or changes that were omitted) that may have impacted the prediction; and the like. In implementations with broad predictions, the feedback can include any of the indications above from numerous responsible persons, feedback from one or more third parties 180 (FIG. 1) reviewing the broad predictions (e.g., corroborating the prediction, suggesting updates to the prediction, contradicting the prediction, suggesting no correlation between correlated events, and the like).

At block 2006, the process 2000 includes aggregating the feedback on the predictions. The aggregation can include linking associated indications (e.g., related to a specific recommended change, a specific predicted reaction, and the like). In some implementations, the aggregation can weight the feedback received. Purely by way of example, for broad predictions, feedback received from a research institution can be given greater weight than feedback from a responsible person.

At block 2008, the process 2000 includes updating the predictive model based on the aggregated feedback. Purely by way of example, where the aggregated feedback indicates that one recommended change had negative impacts on supervised persons, the predictive model can be updated to remove the change from possible recommendations (or limit the instances the change can recommended). Additionally, or alternatively, the process 2000 can include studying any target data associated with the negative feedback to try to understand the divergence between a predicted impact and observed impact to update the predictive model (e.g., thereby treating the feedback similarly to a validation data set).

### Suitable Computer Environments

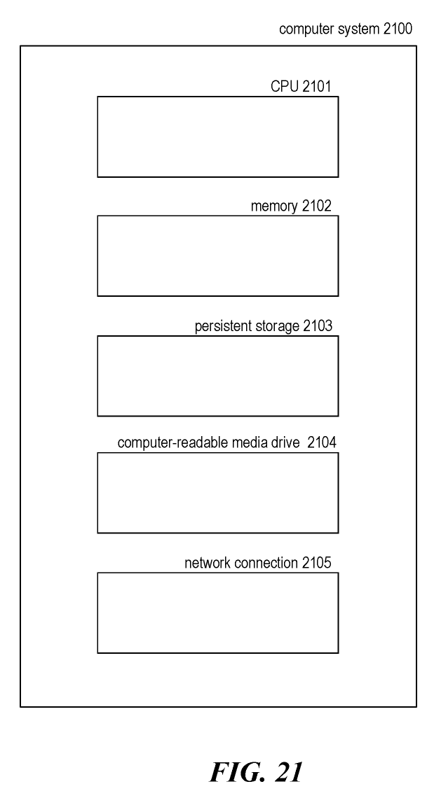

FIG. 21 is a block diagram showing some of the components typically incorporated in at least some of the computer systems and other devices on which the disclosed system operates. In various implementations, these computer systems and other devices 2100 can include server computer systems, desktop computer systems, laptop computer systems, netbooks, mobile phones, personal digital assistants, televisions, cameras, unmanned aerial vehicle computers, aerial vehicle computers, satellite computers, electronic media players, etc. In various implementations, the computer systems and devices include zero or more of each of the following: a central processing unit (CPU) 2101 for executing computer programs; a computer memory 2102 for storing programs and data while they are being used, including the facility and associated data, an operating system including a kernel, and device drivers; a persistent storage device 2103, such as a hard drive or flash drive for persistently storing programs and data; computer-readable media drives 2104 that are tangible storage means that do not include a transitory, propagating signal, such as a floppy, CD-ROM, or DVD drive, for reading programs and data stored on a computer-readable medium; and a network connection 2105 for connecting the computer system to other computer systems to send and/or receive data, such as via the Internet or another network and its networking hardware, such as switches, routers, repeaters, electrical cables and optical fibers, light emitters and receivers, radio transmitters and receivers, and the like. While computer systems configured as described above are typically used to support the operation of the facility, those skilled in the art will appreciate that the facility may be implemented using devices of various types and configurations, and having various components.

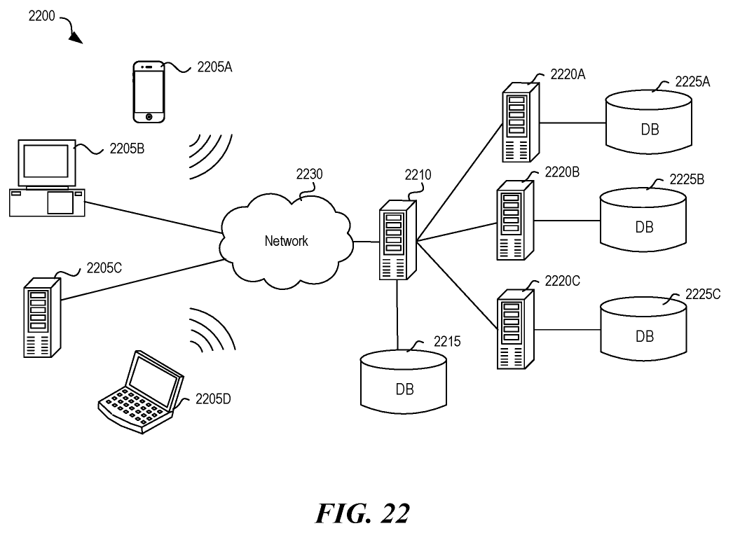

FIG. 22 is a system diagram illustrating an example of a computing environment in which the disclosed system operates in some implementations of the present technology. In some implementations, environment 2200 (sometime also referred to as “system 2200”) includes one or more client computing devices 1405A-D, examples of which can host the system 2200. Client computing devices 2205 operate in a networked environment using logical connections through network 2230 to one or more remote computers, such as a server computing device.

In some implementations, server 2210 is an edge server which receives client requests and coordinates fulfillment of those requests through other servers, such as servers 1420A-C. In some implementations, server computing devices 2210 and 2220 comprise computing systems, such as the system 2200. Though each server computing device 2210 and 2220 is displayed logically as a single server, server computing devices can each be a distributed computing environment encompassing multiple computing devices located at the same or at geographically disparate physical locations. In some implementations, each server 2220 corresponds to a group of servers.

Client computing devices 2205 and server computing devices 2210 and 2220 can each act as a server or client to other server or client devices. In some implementations, servers (2210, 1420A-C) connect to a corresponding database (2215, 1425A-C). As discussed above, each server 2220 can correspond to a group of servers, and each of these servers can share a database or can have its own database. Databases 2215 and 2225 warehouse (e.g., store) information such as home information, biomass measurements, image measurements, carbon estimates, and so on. Though databases 2215 and 2225 are displayed logically as single units, databases 2215 and 2225 can each be a distributed computing environment encompassing multiple computing devices, can be located within their corresponding server, or can be located at the same or at geographically disparate physical locations.

Network 2230 can be a local area network (LAN) or a wide area network (WAN), or other wired or wireless networks. In some implementations, network 2230 is the Internet or some other public or private network. Client computing devices 2205 are connected to network 2230 through a network interface, such as by wired or wireless communication. While the connections between server 2210 and servers 2220 are shown as separate connections, these connections can be any kind of local, wide area, wired, or wireless network, including network 2230 or a separate public or private network.

### EXAMPLES

Various examples of aspects of the present technology are described in the examples discussed below. These are provided as examples and do not limit the present technology. Further, it is noted that the features of the examples can be combined in any suitable manner unless otherwise discussed herein.

In some aspects, the techniques described herein relate to a non-transitory computer-readable storage medium storing instructions that, when executed by a computing system, cause the computing system to perform operations related to monitoring a developmental status of a supervised person, the operations including: receiving, from an electronic device associated with a controlling person, an evaluation of the supervised person based on an interaction between the controlling person and the supervised person; receiving, from a wearable device associated with the supervised person, bioindicator data of the supervised person during the interaction, wherein the bioindicator data is measured by one or more sensors on the wearable device, and wherein the bioindicator data reflects an objective status of the supervised person during the interaction; checking for contradictions between the evaluation and the bioindicator data; responsive to no contradiction being found, generating a new care receiver (CR) rating associated with the interaction based at least partially on the evaluation and the bioindicator data; retrieving one or more past CR ratings associated with past interactions involving the supervised person; and determining whether a sufficient number of total CR ratings exist to evaluate the developmental status of the supervised person, wherein: responsive to a sufficient number of the total CR ratings, the operations further include: evaluating the developmental status of the supervised person based at least partially on each CR rating in the total number of CR ratings; and outputting the developmental status of the supervised person.

In some aspects, the bioindicator data includes measurements of one or more of: heart rate, skin temperature, skin conductivity, movement of the supervised person during the interaction, heart-rate variability, resting heart rate, sweat chemical composition, hydration levels, nervous system electrical signals, stress levels, blood oxygen and/or pulse oxygen, or cardiac system electrical signals.

In some aspects, the new CR rating is further based at least partially on a CR baseline associated with the supervised person, wherein the CR baseline is generated from a weighted average of the one or more past CR ratings to reflect a typical interaction with the supervised person.

In some aspects, the operations further include retrieving an evaluator baseline for the controlling person associated with the electronic device, wherein the evaluator baseline is generated from a weighted average of past evaluations from the controlling person to account for variances in evaluators, and wherein the new CR rating is further based at least partially on the evaluator baseline.

In some aspects, responsive to a contradiction being found between the evaluation and the bioindicator data, the operations further include sending, to the electronic device, a notification of the contradiction, wherein the notification prompts the controlling person to provide an explanation for the contradiction, and wherein the new CR rating is further based at least partially on the explanation.

In some aspects, responsive to a contradiction being found between the evaluation and the bioindicator data, the operations further include sending, to the electronic device, a notification of the contradiction, wherein the notification prompts the controlling person to provide a new evaluation of the supervised person for use in generating the new CR rating.

In some aspects, generating the new CR rating includes associating the new CR rating with a confidence level for the new CR rating, wherein the confidence level is reflective of how likely the new CR rating is to be accurate based on one or more of: the evaluation, the bioindicator data, a CR baseline for the supervised person, an evaluator baseline for the controlling person, whether a contradiction is identified between the evaluation and the bioindicator data, or a qualification status for the controlling person.

In some aspects, evaluating the developmental status is further based at least partially on at least one of: one or more developmental milestones indicated as achieved in the evaluation, one or more expected milestones for the supervised person, one or more developmental classifications indicated in the evaluation, or one or more expected classifications indicated in the evaluation.

In some aspects, the operations further include: detecting the interaction based on proximity signals received from the electronic device indicating a presence of the wearable device within a predetermined distance of the electronic device, wherein the proximity signals are generated based on communication between the electronic device and the wearable device using shortrange wireless communication components; and sending a notification to the electronic device to prompt the controlling person to provide the evaluation of the supervised person during the interaction.

In some aspects, the techniques described herein relate to a non-transitory computer-readable storage medium storing instructions that, when executed by a computing system, cause the computing system to perform operations related to assessing an impact of a controlling person on a supervised person, the operations including: receive, from an electronic device associated with a controlling person, an evaluation of the supervised person based on an interaction between the controlling person and the supervised person; receive, from a wearable device associated with the supervised person, bioindicator data of the supervised person during the interaction; generating a new care receiver (CR) rating associated with the interaction based at least partially on the evaluation and the bioindicator data, wherein the bioindicator data is measured by one or more sensors on the wearable device, and wherein the bioindicator data reflects an objective status of the supervised person during the interaction; retrieving a CR baseline for the supervised person, wherein the CR baseline is generated from a weighted average of past CR ratings for the supervised person and indicative of one or more expectations for assessment values in the evaluation of the supervised person; and generating a new care giver (CG) rating for the supervised person based at least partially on the evaluation of the supervised person and the CR baseline, wherein the new CG rating is at least partially indicative of a reaction of the supervised person to the controlling person.

In some aspects, the techniques described herein relate to a non-transitory computer-readable storage medium wherein the operations further include: checking for contradictions between the evaluation and the bioindicator data; and when a contradiction is found, sending, to the electronic device, a notification of the contradiction, wherein the notification prompts the controlling person to provide a new evaluation of the supervised person for use in generating the new CR rating.

In some aspects, the techniques described herein relate to a non-transitory computer-readable storage medium wherein the new CG rating is further based at least partially on the bioindicator data from the wearable device.

In some aspects, the techniques described herein relate to a non-transitory computer-readable storage medium wherein the operations further include: retrieving past CG ratings for the controlling person; updating a CG baseline for the controlling person, wherein the CG baseline is generated from a weighted average of the past CG ratings for the controlling person and indicative of one or more expectations for assessment values in the evaluation of the supervised person from the controlling person; determining whether a sufficient number of total CG ratings to account for fluctuations in the evaluation specific to the controlling person; and responsive to a sufficient number of the total CG ratings, the operations further include: generating a rated interpersonal interaction (RIPI) score for the controlling person, wherein the RIPI score is indicative of a developmental impact of the controlling person on the supervised person; and output the RIPI score.

In some aspects, the RIPI score is based at least partially on a comparison of received data in the evaluation of the supervised person and expected data.

In some aspects, the expected data is based on one or more of: World Health Organization classifications for development, or Centers for Disease Control and Prevention developmental milestones.

In some aspects, retrieving the CR baseline includes retrieving a record of the CR baseline over time, and wherein the RIPI score is based at least partially on a change in the CR baseline reflected in the record.

In some aspects, the bioindicators are at least partially indicative of an emotional state of the supervised person during the interaction, and wherein the new CG rating is further based at least partially on the emotional state of the supervised person during the interaction.

In some aspects, the techniques described herein relate to a method for improving an assessment of a developmental status of a supervised person, the method including: receiving, from a first electronic device associated with a first controlling person, a first evaluation of the supervised person based on an interaction between the first controlling person and the supervised person; receiving, from a second electronic device associated with a second controlling person, a second evaluation of the supervised person based on the interaction; receiving, from a wearable device associated with the supervised person, bioindicator data of the supervised person during the interaction, wherein the bioindicator data is measured by one or more sensors on the wearable device, and wherein the bioindicator data reflects an objective status of the supervised person during the interaction; checking for contradictions between the first evaluation and the bioindicator data; checking for contradictions between the second evaluation and the bioindicator data; responsive to no contradictions being found, generating a new care receiver (CR) rating associated with the interaction based at least partially on the first evaluation, the second evaluation, and the bioindicator data; retrieving one or more past CR ratings associated with past interactions involving the supervised person; and determining whether a sufficient number of total CR ratings exist to evaluate the developmental status of the supervised person, wherein: responsive to a sufficient number of the total CR ratings, the method further includes: evaluating the developmental status of the supervised person based at least partially on each CR rating in the total number of CR ratings; and outputting the developmental status of the supervised person.

In some aspects, responsive to a first contradiction being found between the first evaluation and the bioindicator data, the method further includes sending, to the first electronic device, a notification of the first contradiction, wherein the notification prompts the first controlling person to provide an explanation for the first contradiction, and wherein the new CR rating is further based at least partially on the explanation; and responsive to a second contradiction being found between the second evaluation and the bioindicator data, the method further includes sending, to the second electronic device, a notification of the second contradiction, wherein the notification prompts the second controlling person to explain the second contradiction, and wherein the new CR rating is further based at least partially on the explanation.

In some aspects, the method further includes checking for contradictions between the first evaluation and the second evaluation; and responsive to an evaluation contradiction being found between the first evaluation and the second evaluation, the method further includes sending, to the first electronic device, a notification of the evaluation contradiction, wherein the notification prompts the first controlling person to update the first evaluation and/or explain the contradiction.

In some aspects, the techniques described herein relate to a wearable device for monitoring a developmental status of a supervised person, the wearable device including: a housing; one or more sensors carried by the housing and positioned to measure one or more bioindicators of the supervised person, wherein each of the bioindicators reflect an objective psychological and/or physiological status of the supervised person; and an operating platform implemented a processor within the electronics housing, wherein the operating platform includes one or more modules to control the wearable device to: detect an interaction between the supervised person and a responsible person; communicate, to a remote server, bioindicator data from the one or more sensors during the detected interaction.

In some aspects, the one or more sensors include at least one of: a PPG sensor, an accelerometer, a skin temperature sensor, a skin conductivity sensor, additional hydration sensors, heart-rate variability sensors, resting heart rate sensors, sweat chemical composition sensors, nervous system electrical sensors, air quality sensors, UV exposure sensors, sensors to detect environmental chemicals, blood oxygen and/or pulse oxygen sensors, voice recognition, electrocardiogram (ECG) sensor, pressure sensors, gyroscopes, or magnetometers.

In some aspects, the wearable device, further includes a shortrange wireless component communicably couplable to a remote subsystem associated with the responsible person; and at least one long range communication component communicably couplable to the remote server.

In some aspects, the operating platform is further configured to: receive the bioindicator data from the one or more sensors; link corresponding portions of the bioindicator data based on a time of measurement; and store the linked bioindicator date.

In some aspects, the operating platform is further configured to: receive the bioindicator data from the one or more sensors; and process the bioindicator data to make one or more determinations on the psychological and/or physiological status of the supervised person.

In some aspects, the operating platform is further configured to: detect, based on the bioindicator data from the one or more sensors, a stress event experienced by the supervised person; and in response to the detected stress event, control the one or more sensors to collect additional bioindicator data surrounding the detected stress event.

In some aspects, in response to the detected stress event, the operating platform is further configured to send a notification to the responsible person, the notification including an indication of the stress event and a prompt for an explanation of the stress event.

In some aspects, the operating platform is further configured to: receive, from the remote server, a request for bioindicator data outside of the detected interaction; and in response to the received request: retrieve, from one or more memories on the wearable device, the requested bioindicator data; and send, to the remote server, the bioindicator requested data.

In some aspects, detecting the interaction includes receiving one or more presence detection signals from a subsystem on an electronic device associated with the responsible person, wherein the one or more presence detection signals indicate that the responsible person is within a predetermined vicinity of the supervised person. In some aspects, detecting the interaction includes: sending one or more presence detection signals configured to identify a subsystem on an electronic device associated with the responsible person within a predetermined vicinity of the supervised person; and receiving a response to the one or more presence detection signals from the subsystem on the electronic device associated with the responsible person.

In some aspects, the techniques described herein relate to a system for assessing a developmental status of a supervised person, the system including: a first subsystem having at least one first communication component and configured to receive an evaluation of the supervised person; a second subsystem having at least one second communication component and configured to obtain and communicate bioindicator data from the supervised person; and a remote server having at least one database in communication with the first communication component and the second communication component, wherein the remote server includes a module configured to: receive a new CR rating for the supervised person; determine whether the remote server has a sufficient database of CR ratings to evaluate the supervised person; when the remote server does not have the sufficient database of CR ratings, update a CR baseline for the supervised person; and when the remote server does have the sufficient database of CR ratings: update the CR baseline for the supervised person; evaluate the developmental status of the supervised person based at least partially on each CR rating in the database of CR ratings and/or the CR baseline; and output the developmental status.

In some aspects, the techniques described herein relate to a system wherein module is a first module, and wherein the remote server includes a second module configured to: receive the evaluation of the supervised person from the first subsystem; receive the bioindicator data from the second subsystem; generate the new CR rating for the supervised person based at least partially on the evaluation of the supervised person and the bioindicator data; and send, to the first module the new CR rating.

In some aspects, the techniques described herein relate to a system wherein the second module is further configured to: check for contradictions between the evaluation of the supervised person and the bioindicator data; and when a contradiction is found, send, to the first subsystem, a notification of the contradiction, wherein the notification prompts a user of the first subsystem to provide a new evaluation of the supervised person.

In some aspects, the techniques described herein relate to a system wherein the second module is further configured to: check for contradictions between the evaluation of the supervised person and the bioindicator data; and when a contradiction is found, send, to the first subsystem, a notification of the contradiction, wherein the notification prompts a user of the first subsystem to explain found the contradiction.

In some aspects, the techniques described herein relate to a system wherein evaluating the developmental status is based at least partially on one or more of the following: the CR baseline, average values in data from one or more CR ratings, average values in the bioindicator data, achieved milestones indicated in the evaluation, classifications indicated in the evaluation, expected milestones for the supervised person, expected classifications indicated in the evaluation, changes in the average values in the data from one or more CR ratings over time, changes in the CR baseline over time, and adjustment factors for a supervising person providing the evaluation.

In some aspects, the techniques described herein relate to a system for rating a supervised person during an interpersonal interaction, the system including: a first subsystem having at least one first communication component and configured to receive an evaluation of the supervised person; a second subsystem having at least one second communication component and configured to obtain and communicate bioindicator data from the supervised person; and a remote server having at least one database in communication with the first communication component and the second communication component, wherein the remote server includes a module configured to: receive the bioindicators from the second subsystem after the interpersonal interaction; receive the evaluation of the supervised person from the first subsystem after the interpersonal interaction; check for contradictions between the evaluation of the supervised person and the bioindicator data; and when no contradiction is found, generate a CR rating for the supervised person based at least partially on the evaluation of the supervised person and the bioindicator data.

In some aspects, the techniques described herein relate to a system wherein the module is further configured to, when a contradiction is found, send, to the first subsystem, a notification of the contradiction, wherein the notification prompts a user of the first subsystem to provide a new evaluation of the supervised person.

In some aspects, the techniques described herein relate to a system wherein the module is further configured to, when a contradiction is found, send, to the first subsystem, a notification of the contradiction, wherein the notification prompts a user of the first subsystem to provide an explanation for the found contradiction.

In some aspects, the techniques described herein relate to a system for rating a supervising person during an interpersonal interaction, the system including: a first subsystem having at least one first communication component and configured to receive an evaluation of a supervised person; a second subsystem having at least one second communication component and configured to obtain and communicate bioindicator data from the supervised person; and a remote server having at least one database in communication with the first communication component and the second communication component, wherein the remote server includes a module configured to: receive the bioindicators from the second subsystem after the interpersonal interaction; receive the evaluation of the supervised person from the first subsystem after the interpersonal interaction; retrieve a CR baseline for the supervised person, the CR baseline at least partially indicating expectations for assessment values in the evaluation of the supervised person; and generate a CG rating for the supervised person based at least partially on the evaluation of the supervised person and the CR baseline.

In some aspects, the techniques described herein relate to a system, wherein the module is further configured to: check for contradictions between the evaluation of the supervised person and the bioindicator data; and when a contradiction is found, send, to the first subsystem, a notification of the contradiction, wherein the notification prompts a user of the first subsystem to provide a new evaluation of the supervised person.

In some aspects, the techniques described herein relate to a system, wherein the module is further configured to: check for contradictions between the evaluation of the supervised person and the bioindicator data; and when a contradiction is found, send, to the first subsystem, a notification of the contradiction, wherein the notification prompts a user of the first subsystem to explain the found contradiction.

In some aspects, the techniques described herein relate to a system for assessing the developmental impact of a supervising person on a supervised person, the system including: a first subsystem having at least one first communication component and configured to receive an evaluation of a supervised person; a second subsystem having at least one second communication component and configured to obtain and communicate bioindicator data from the supervised person; and a remote server having at least one database in communication with the first communication component and the second communication component, wherein the remote server includes a module configured to: receive a CG rating for the supervising person; determine whether the remote server has a sufficient database of CG ratings to evaluate the supervising person; when the remote server does not have the sufficient database of CG ratings, update a CG baseline for the supervising person; and when the remote server does have the sufficient database of CG ratings: update the CG baseline for the supervising person; generate a rated interpersonal interaction score (RIPI score) for the supervising person, wherein the RIPI score is indicative of the developmental impact of the supervising person on the supervised person; and output the RIPI score.

In some aspects, the techniques described herein relate to a system wherein the RIPI score is at least partially dependent on one or more of the following: the CG baseline, average values in stored evaluations from the supervising person, average values for contradiction metrics between the stored evaluations and stored bioindicators, changes in supervised person-specific values in the stored evaluations for a plurality of supervised persons over time, changes in a CR baseline for the plurality of supervised persons over time, changes in the supervised person-specific developmental statuses for the plurality of supervised persons over time, and reviews from one or more controlling persons associated with the plurality of supervised persons.

In some aspects, the techniques described herein relate to a system wherein the module is further configured to: check for contradictions between the evaluation of the supervised person and the bioindicator data; and when a contradiction is found, send, to the first subsystem, a notification of the contradiction, wherein the notification prompts a user of the first subsystem to provide a new evaluation of the supervised person.

In some aspects, the techniques described herein relate to a system wherein the module is further configured to: check for contradictions between the evaluation of the supervised person and the bioindicator data; and when a contradiction is found, send, to the first subsystem, a notification of the contradiction, wherein the notification prompts a user of the first subsystem to explain the found contradiction.

In some aspects, the techniques described herein relate to a system wherein the module is further configured to: check for contradictions between the evaluation of the supervised person and the bioindicator data; and generate data related to a number of contradictions found and a quality of the contradictions found.

In some aspects, the techniques described herein relate to a system wherein the second subsystem is housed in a wearable device on the supervised person, and wherein the second subsystem includes one or more sensors positioned to gather the bioindicator data.

In some aspects, the techniques described herein relate to a system for evaluating a developmental status of a supervised person the system including: a first subsystem having at least one first communication component and configured to receive an evaluation of having developmental data about the supervised person; a second subsystem having at least one second communication component and configured to obtain bioindicator data from the supervised person; and a remote server having at least one database in communication with the first communication component and the second communication component, wherein the remote server includes a module configured to: receive the developmental data and the bioindicator data; apply a predictive model to the developmental data and the bioindicator data; and output a result from the predictive model, wherein the result includes an assessment of the developmental status of the supervised person based on the developmental data and the bioindicator data.

In some aspects, the module on the system is further configured to: before applying the predictive model to the developmental data and the bioindicator data, retrieve training data related to the supervised person; apply an artificial intelligence or machine learning (AI/ML) algorithm to the training data related to the supervised person to adjust the predictive model for use on the developmental data and the bioindicator data for the supervised person; and output the adapted predictive model, wherein the module is configured to apply the adapted predictive model to the developmental data and the bioindicator data.

In some aspects, the module is a first module, wherein the remote server includes a database storing target data related to a N-number of reference persons, and wherein the remote server includes a second module configured to: for each reference person, generate one or more classes for the target data and label the target data into the one or more classes; aggregate the target data in each of the one or more classes; sort the target data into sets that include at least a training set and a testing set; apply an artificial intelligence or machine learning (AI/ML) algorithm to the training set to generate a candidate predictive model; apply the candidate predictive model to the testing set to evaluate the candidate predictive model; and when the candidate predictive model is satisfactory, output the candidate predictive model as the predictive model, or when the candidate predictive model is not satisfactory, re-apply the AI/ML algorithm to the training set to generate a new candidate predictive model.

In some aspects, the first module is further configured to: check for contradictions between the developmental data and the bioindicator data; and when a contradiction is found, send, to the first subsystem, a notification of the contradiction, wherein the notification prompts a user of the first subsystem to provide a new evaluation of the supervised person.

In some aspects, the system further includes: a third subsystem having at least one third communication component and configured to receive an additional evaluation of having additional developmental data about the supervised person, wherein the module is further configured to: receive the additional developmental data; and use the additional developmental data to generate or update the assessment of the developmental status of the supervised person.

In some aspects, the techniques described herein relate to a system for predicting how one or more changes will impact a developmental status of a supervised person, the system including: a first subsystem having at least one first communication component and configured to receive an evaluation of having developmental data about the supervised person; a second subsystem having at least one second communication component and configured to obtain bioindicator data from the supervised person; and a remote server having at least one database in communication with the first communication component and the second communication component, wherein the remote server includes a module configured to: receive the developmental data and the bioindicator data; receive an indication of the one or more changes; apply a predictive model to the developmental data, the bioindicator data, and the one or more changes; and output a result from the predictive model, wherein the result includes one or more predictions of how the developmental status of the supervised person will be impacted by the one or more changes.

In some aspects, the one or more predictions includes a prediction of how a behavior of the supervised person will be impacted by the one or more changes. In some aspects, the one or more predictions includes a prediction of how a developmental status of the supervised person will be impacted over time by the one or more changes.

In some aspects, the module is further configured to: before applying the predictive model to the developmental data and the bioindicator data, retrieve training data related to the supervised person; apply an artificial intelligence or machine learning (AI/ML) algorithm to the training data related to the supervised person to adjust the predictive model for use on the developmental data and the bioindicator data for the supervised person; and output the adapted predictive model, wherein the module is configured to apply the adapted predictive model to the developmental data and the bioindicator data.

In some aspects, the module is a first module, wherein the remote server includes a database storing target data related to a N-number of reference persons, and wherein the remote server includes a second module configured to: for each reference person, generate one or more classes for the target data and label the target data into the one or more classes; aggregate the target data in each of the one or more classes; sort the target data into sets that include at least a training set and a testing set; apply an artificial intelligence or machine learning (AI/ML) algorithm to the training set to generate a candidate predictive model; apply the candidate predictive model to the testing set to evaluate the candidate predictive model; and when the candidate predictive model is satisfactory, output the candidate predictive model as the predictive model, or when the candidate predictive model is not satisfactory, re-apply the AI/ML algorithm to the training set to generate a new candidate predictive model.

In some aspects, the first module is further configured to: check for contradictions between the developmental data and the bioindicator data; and when a contradiction is found, send, to the first subsystem, a notification of the contradiction, wherein the notification prompts a user of the first subsystem to provide a new evaluation of the supervised person.

In some aspects, the system further includes: a third subsystem having at least one third communication component and configured to receive an additional evaluation of having additional developmental data about the supervised person, wherein the module is further configured to: receive the additional developmental data; and use the additional developmental data to generate or update the one or more predictions of how the developmental status of the supervised person will be impacted by the one or more changes.

In some aspects, the techniques described herein relate to a system for generating recommendations for one or more changes to impact a developmental status of a supervised person, the system including: a first subsystem having at least one first communication component and configured to receive an evaluation of having developmental data about the supervised person; a second subsystem having at least one second communication component and configured to obtain bioindicator data from the supervised person; and a remote server having at least one database in communication with the first communication component and the second communication component, wherein the remote server includes a module configured to: receive the developmental data and the bioindicator data; apply a predictive model to the developmental data and the bioindicator data; output a result from the predictive model, wherein the result includes an assessment of the developmental status of the supervised person and an indication of one or more changes to impact the developmental status of the supervised person.

In some aspects, the result from the predictive model further includes a prediction of how the one or more changes will impact the developmental status of the supervised person.

In some aspects, the module is further configured to: before applying the predictive model to the developmental data and the bioindicator data, retrieve training data related to the supervised person; apply an artificial intelligence or machine learning (AI/ML) algorithm to the training data related to the supervised person to adjust the predictive model for use on the developmental data and the bioindicator data for the supervised person; and output the adapted predictive model, wherein the module is configured to apply the adapted predictive model to the developmental data and the bioindicator data.

In some aspects, the module is a first module, wherein the remote server includes a database storing target data related to a N-number of reference persons, and wherein the remote server includes a second module configured to: for each reference person, generate one or more classes for the target data and label the target data into the one or more classes; aggregate the target data in each of the one or more classes; sort the target data into sets that include at least a training set and a testing set; apply an artificial intelligence or machine learning (AI/ML) algorithm to the training set to generate a candidate predictive model; apply the candidate predictive model to the testing set to evaluate the candidate predictive model; and when the candidate predictive model is satisfactory, output the candidate predictive model as the predictive model, or when the candidate predictive model is not satisfactory, re-apply the AI/ML algorithm to the training set to generate a new candidate predictive model.

In some aspects, the first module is further configured to: check for contradictions between the developmental data and the bioindicator data; and when a contradiction is found, send, to the first subsystem, a notification of the contradiction, wherein the notification prompts a user of the first subsystem to provide a new evaluation of the supervised person.

In some aspects, the system further includes: a third subsystem having at least one third communication component and configured to receive an additional evaluation of having additional developmental data about the supervised person, wherein the module is further configured to: receive the additional developmental data; and use the additional developmental data to generate or update the assessment of the developmental status of the supervised person and/or the indication of one or more changes to impact the developmental status of the supervised person.

In some aspects, the techniques described herein relate to a system for aggregating information related to human development, the system including: a remote server having at least one database in communication with the first communication component and the second communication component, wherein the remote server includes a module configured to: receive a set of target data for N-number of supervised persons, each set of target data including at least one of: assessments of the supervised person including developmental data, and bioindicator data related to the supervised person; generate one or more classes for the target data and label the target data into the one or more classes; aggregate the target data in each of the one or more classes; receive a request for target data in at least one class of the one or more classes; and output the target data in the at least one of the one class.

In some aspects, the module is further configured to, for each of the N-number of supervised persons: before classifying and labeling the target data, format the target data into a standardized format; after classifying and labeling the target data, check for associated classes and/or associated data between classes; and if an association is found, generate a link between associated data.

### CONCLUSION

From the foregoing, it will be appreciated that specific implementations of the technology have been described herein for purposes of illustration, but well-known structures and functions have not been shown or described in detail to avoid unnecessarily obscuring the description of the implementations of the technology. To the extent any material incorporated herein by reference conflicts with the present disclosure, the present disclosure controls. Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Furthermore, as used herein, the phrase “and/or” as in “A and/or B” refers to A alone, B alone, and both A and B. Additionally, the terms “comprising,” “including,” “having,” and “with” are used throughout to mean including at least the recited feature(s) such that any greater number of the same features and/or additional types of other features are not precluded.

From the foregoing, it will also be appreciated that various modifications may be made without deviating from the disclosure or the technology. For example, one of ordinary skill in the art will understand that various components of the technology can be further divided into subcomponents, or that various components and functions of the technology may be combined and integrated. In addition, certain aspects of the technology described in the context of particular implementations may also be combined or eliminated in other implementations. Furthermore, although advantages associated with certain implementations of the technology have been described in the context of those implementations, other implementations may also exhibit such advantages, and not all implementations need necessarily exhibit such advantages to fall within the scope of the technology. Accordingly, the disclosure and associated technology can encompass other implementations not expressly shown or described herein.

We claim:

1. A non-transitory computer-readable storage medium storing instructions that, when executed by a computing system, cause the computing system to perform operations related to monitoring developmental statuses of supervised persons, the operations comprising:
detecting a set of interactions based on proximity signals received from an electronic device associated with a controlling person indicating a presence of a wearable device, associated with a supervised person, within a predetermined distance of the electronic device, wherein the proximity signals are generated based on communication between the electronic device and the wearable device using shortrange wireless communication components;
receiving, from the electronic device, an evaluation of the supervised person based on the set of interactions between the controlling person and the supervised person;
receiving, from the wearable device associated with the supervised person, bioindicator data of the supervised person during the set of interactions, wherein the bioindicator data is measured by one or more sensors on the wearable device, and wherein the bioindicator data reflects an objective status of the supervised person during the set of interactions;
performing a comparison of the received evaluation of the supervised person and an expected evaluation of the supervised person generated using the bioindicator data to identify a set of contradictions;
generating a new care receiver (CR) rating associated with the set of interactions based at least partially on a comparison of the received evaluation and an expected evaluation of the supervised person;
retrieving one or more past CR ratings associated with a set of past interactions involving the supervised person;
determining whether a sufficient number of total CR ratings exist to evaluate a developmental status of the supervised person, wherein the total number of CR ratings comprise the one or more past CR ratings and the new generated CR rating; and
responsive to a sufficient number of the total CR ratings existing to evaluate the developmental status of the supervised person:
evaluating the developmental status of the supervised person based at least partially on each CR rating in the total number of CR ratings; and
outputting the developmental status of the supervised person.

2. The non-transitory computer-readable storage medium of claim 1 wherein the bioindicator data includes measurements of one or more of: heart rate, skin temperature, skin conductivity, movement of the supervised person during the set of interactions, heart-rate variability, resting heart rate, sweat chemical composition, hydration levels, nervous system electrical signals, stress levels, blood oxygen and/or pulse oxygen, or cardiac system electrical signals.

3. The non-transitory computer-readable storage medium of claim 1 wherein the new generated CR rating is further based on a CR baseline associated with the supervised person, wherein the CR baseline is generated from a weighted average of the one or more past CR ratings to reflect a typical interaction with the supervised person.

4. The non-transitory computer-readable storage medium of claim 1 wherein the operations further comprise retrieving an evaluator baseline for the controlling person associated with the electronic device, wherein the evaluator baseline is generated from a weighted average of past evaluations from the controlling person to account for variances in evaluators, and wherein the new generated CR rating is further based at least partially on the evaluator baseline.

5. The non-transitory computer-readable storage medium of claim 1 wherein, responsive to a contradiction being found in the set of contradictions, the operations further comprise sending, to the electronic device, a notification of the contradiction, wherein the notification prompts the controlling person to provide an explanation for the contradiction, and wherein the new generated CR rating is further based at least partially on the explanation for the contradiction.

6. The non-transitory computer-readable storage medium of claim 1 wherein, responsive to a contradiction being found in the set of contradictions, the operations further comprise sending, to the electronic device, a notification of the contradiction, wherein the notification prompts the controlling person to provide a new evaluation of the supervised person for use in generating the new generated CR rating.

7. The non-transitory computer-readable storage medium of claim 1 wherein generating the new generated CR rating includes associating the new generated CR rating with a confidence level for the new generated CR rating, wherein the confidence level is reflective of how likely the new generated CR rating is to be accurate based on one or more of: the evaluation, the bioindicator data, a CR baseline for the supervised person, an evaluator baseline for the controlling person, a number of contradictions identified in the set of contradictions, or a qualification status for the controlling person.

8. The non-transitory computer-readable storage medium of claim 1 wherein evaluating the developmental status is further based at least partially on at least one of: one or more developmental milestones indicated as achieved in the evaluation, one or more expected milestones for the supervised person, one or more developmental classifications indicated in the evaluation, or one or more expected classifications indicated in the evaluation.

9. The non-transitory computer-readable storage medium of claim 1 wherein the operations further comprise:
sending a notification to the electronic device to prompt the controlling person to provide the evaluation of the supervised person during the detected set of interactions, wherein the notification includes instructions for providing the evaluation, and wherein the instructions include one or more questions specific to the detected set of interactions.

10. A non-transitory computer-readable storage medium storing instructions that, when executed by a computing system, cause the computing system to perform operations related to assessing an impact of controlling persons on supervised persons, the operations comprising:
receive, from an electronic device associated with a controlling person, an evaluation of a supervised person based on a set of interactions between the controlling person and the supervised person;
receive, from a wearable device of the supervised person, bioindicator data of the supervised person during the set of interactions;
generating a new care receiver (CR) rating associated with the set of interactions based at least partially on the evaluation and the bioindicator data, wherein the bioindicator data is measured by one or more sensors on the wearable device, and wherein the bioindicator data reflects an objective status of the supervised person during the set of interactions;
retrieving a CR baseline for the supervised person, wherein the CR baseline is generated from a weighted average of past CR ratings for the supervised person and indicative of one or more expectations for assessment values in the evaluation of the supervised person; and
generating a new care giver (CG) rating for the controlling person based at least partially on the evaluation of the supervised person and the CR baseline, wherein the new CG rating is at least partially indicative of a reaction of the supervised person to the controlling person during the set of interactions.

11. The non-transitory computer-readable storage medium of claim 10 wherein the operations further comprise:
performing a comparison of the received evaluation of the supervised person and an expected evaluation of the supervised person generated using the bioindicator data to identify a set of contradictions; and
when a contradiction is found in the set of contradictions, sending, to the electronic device, a notification of the contradiction, wherein the notification prompts the controlling person to provide a new evaluation of the supervised person for use in generating the new CR rating.

12. The non-transitory computer-readable storage medium of claim 10 wherein the operations further comprise detecting the set of interactions based on GPS data received from the electronic device and the wearable device indicating that the controlling person and the supervised person were within a predetermined distance of each other.

13. The non-transitory computer-readable storage medium of claim 10 wherein the operations further comprise:
retrieving past CG ratings for the controlling person;
updating a CG baseline for the controlling person, wherein the CG baseline is generated from a weighted average of the past CG ratings for the controlling person and indicative of one or more expectations for assessment values in the evaluation of the supervised person from the controlling person;
determining whether a sufficient number of total CG ratings to account for fluctuations in the evaluation specific to the controlling person, wherein the total number of CG ratings comprise the past CG ratings and the new CG rating; and
responsive to a sufficient number of the total CG ratings, the operations further comprise:
generating a rated interpersonal interaction (RIPI) score for the controlling person, wherein the RIPI score is indicative of a developmental impact of the controlling person on the supervised person; and
output the RIPI score.

14. The non-transitory computer-readable storage medium of claim 13 wherein the RIPI score is based at least partially on a comparison of received data in the evaluation of the supervised person and expected data.

15. The non-transitory computer-readable storage medium of claim 14 wherein the expected data is based on one or more of: World Health Organization classifications for development, or Centers for Disease Control and Prevention developmental milestones.

16. The non-transitory computer-readable storage medium of claim 13 wherein retrieving the CR baseline includes retrieving a record of the CR baseline over time, and wherein the RIPI score is based at least partially on a change in the CR baseline reflected in the record.

17. The non-transitory computer-readable storage medium of claim 10 wherein the bioindicators are at least partially indicative of an emotional state of the supervised person during the set of interactions, and wherein the new CG rating is further based at least partially on the emotional state of the supervised person during the set of interactions.

18. A wearable device for monitoring a developmental status of a supervised person, the wearable device comprising:
a housing;
one or more sensors carried by the housing and positioned to measure one or more bioindicators of the supervised person, wherein each of the bioindicators reflect an objective status of the supervised person; and
an operating platform implemented at a processor within the housing, wherein the operating platform comprises one or more modules to control the wearable device to:
detect an interaction between the supervised person and a responsible person; and
communicate, to a remote server, data comprising: bioindicator data collected from the one or more sensors during the detected interaction, and data identifying the detected interaction.

19. The wearable device of claim 18, wherein the operating platform is further configured to:
detect, based on the bioindicator data from the one or more sensors, a stress event experienced by the supervised person; and
in response to the detected stress event:
control the one or more sensors to collect additional bioindicator data surrounding the detected stress event; and
send a notification to the responsible person, the notification including an indication of the stress event and a prompt for an explanation of the stress event.

20. The wearable device of claim 18 wherein detecting the interaction includes:
sending one or more presence detection signals configured to identify a subsystem on an electronic device associated with the responsible person within a predetermined vicinity of the supervised person; and
receiving a response to the one or more presence detection signals from the subsystem on the electronic device associated with the responsible person.
